(19) United States
(12) Patent Application Publication
(10) Pub. No.: US 2007/0048325 A1
(43) Pub. Date: Mar. 1, 2007

(54) Combination therapies for inhibiting integrin-extracellular matrix interactions

(76) Inventors: Dennis Van Epps, San Juan Capistrano, CA (US); Bruce Freimark, Laguna Niguel, CA (US); Peter C. Brooks, Carmel, NY (US)

Correspondence Address: WILSON SONSINI GOODRICH & ROSATI, 650 PAGE MILL ROAD, PALO ALTO, CA, 94304-1050, US

(21) Appl. No.: 11/508,754

(22) Filed: Aug. 22, 2006

Related U.S. Application Data
(60) Provisional application No. 60/711,060, filed on Aug. 24, 2005.

(51) Int. Cl. A61K 39/395

(52) U.S. Cl. 424/155.1

(57) ABSTRACT

The present application relates to compositions of ECM antagonists and their use in methods for treating angiogenic-dependent conditions. The ECM antagonists can bind the same or different ECM component. The present application also relates to compositions of ECM antagonists and cancer therapies and their use in methods for preventing, treating or managing angiogenic dependent conditions such as cancer.

### RELATED APPLICATIONS

This application claims the benefit of U.S. Provisional Application No. 60/711,060, filed Aug. 24, 2005, entitled “Combination Therapies for Inhibiting Integrin-Extracellular Matrix Interactions,” which is hereby incorporated in its entirety by reference.

### FIELD OF THE INVENTION

The present application relates to the field of medicine, specifically to methods and compositions for inhibiting angiogenesis and other processes important in tumor metastasis, based on the use of combination therapies that inhibit interactions between integrins and extracellular matrix components.

### BACKGROUND OF THE INVENTION

Identification of proteins involved in tumor cell interactions with the proteolytically-remodeled ECM can provide novel therapeutic targets and treatment strategies for treating malignant tumors. While many studies have confirmed the importance of targeting specific secreted growth factors, proteases, cell surface adhesion receptors and intracellular regulatory molecules, the success of these approaches has been limited due in part to the genetic instability of tumor cells (Molife, et al., Crit. Rev. Oncol. Hematol. 2002, 44:81-102; Brown, et al., Melanoma 2001, 3:344-352; Soengas, et al., Oncogene 2003, 22:3138-3151; Masters, et al., Nat. Rev. Cancer 2003, 3:517-525). Therefore, identifying new functional targets within the non-cellular compartment provides a promising clinical strategy. Metastasis, or the spread of malignant tumor cells from the primary tumor mass to distant sites, involves a complex series of interconnected events. Understanding the biochemical, molecular, and cellular processes that regulate tumor metastasis are of great importance to treating these tumors. The metastatic cascade is thought to be initiated by a series of biochemical and genetic alterations leading to changes in cell-cell interactions allowing disassociation of cells from the primary tumor mass. These events are followed by local invasion and migration through the proteolytically-remodeled extracellular matrix (ECM) to allow access of the tumor cells to the host circulation. In order to establish secondary metastatic deposits, the malignant cells evade the host immune surveillance, arrest in the microvasculature and extravasate out of the circulation. Finally, circulating tumor cells can adhere to the ECM in a new location, proliferate, and recruit new blood vessels by induction of angiogenesis, thereby forming secondary metastatic foci (Liotta, et al., Cell 1991, 64:327-336; Wyckoff, et al., Cancer Res. 2000, 60:2504-2511; Kurschat, et al., Clin. Exp. Dermatol. 2000, 25:482-489; Pantel, et al., Nat. Rev. Cancer 2004, 4:448-456; Hynes, et al., Cell 2003, 113:821-823; Bashyam, M. D., Cancer 2002, 94:1821-1829).

The ECM is an interconnected molecular network that not only provides mechanical support for cells and tissues, but also regulates biochemical and cellular processes such as adhesion, migration, gene expression and differentiation. Extracellular matrix components include, e.g., collagen, fibronectin, osteopontin, laminin, fibrinogen, elastin, thrombospondin, tenascin and vitronectin.

Cryptic sites, including those recognized by antibodies such as HUIV26 and HUIV77, within collagen regulate angiogenesis and endothelial cell behavior (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210). This functional cryptic site was shown to be highly expressed within the ECM of malignant tumors and within the sub-endothelial basement membrane of tumor-associated blood vessels, and its exposure found to be involved in the regulation of angiogenesis in vivo (Xu, et al., Hybridoma 2000, 19:375-385; Xu, et al., J. Cell Biol. 2001, 154:1069-1079; Hangai, et al., Am. J. Pathol. 2002, 161:1429-1437; Lobov, et al., Proc. Natl. Acad. Sci. USA 2002, 99:11205-11210, and U.S. Ser. No. 09/478,977, now U.S. Pub. No. 2003/0113331, the disclosure of which is incorporated herein by reference in its entirety).

Cryptic sites in the ECM component, laminin, have also been described, e.g., in U.S. Publication No. 2004/224896 A1 (the disclosure of which is incorporated herein by reference in its entirety), and WO 2004/087734.

There are potentially important cryptic epitopes in other ECM proteins, e.g., fibronectin (Hocking, et al., J. Cell. Biol. 2002, 158:175-184), fibrinogen (Medved et al., Ann. N.Y. Acad. Sci. 2001, 936:185-204), and osteopontin (Yamamoto, et al., J. Clin. Invest. 2003, 12:181-188).

Angiogenesis is the physiological process by which new blood vessels develop from pre-existing vessels (Varner, et al., Cell Adh. Commun. 1995, 3:367-374; Blood, et al., Biochim. Biophys. Acta. 1990, 1032:89-118; Weidner, et al., J. Natl. Cancer Inst. 1992, 84:1875-1887). Angiogenesis has been suggested to play roles in both normal and pathological processes. For example, angiogenic processes are involved in the development of the vascular systems of animal organs and tissues. They are also involved in transitory phases of angiogenesis, for example during the menstrual cycle, in pregnancy, and in wound healing. On the other hand, a number of diseases are known to be associated with deregulated angiogenesis.

In certain pathological conditions, angiogenesis is recruited as a means to provide adequate blood and nutrient supply to the cells within the affected tissue. Many of these pathological conditions involve aberrant cell proliferation or regulation. Therefore, inhibition of angiogenesis is a potentially useful approach to treating diseases that are characterized by unregulated blood vessel development. For example, angiogenesis is involved in pathologic conditions including: ocular diseases, e.g., macular degeneration, neovascular glaucoma, retinopathy of prematurity and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; vitamin A deficiency; Sjorgen's disease; acne rosacea; mycobacterium infections; bacterial and fungal ulcers; Herpes simplex infections; systemic lupus; retrolental fibroplasia; rubeosis; capillary proliferation in atherosclerotic plaques, and osteoporosis. Angiogenesis is also involved in cancer-associated disorders, including, for example, solid tumors, tumor metastases, blood borne tumors such as leukemias, angiofibromas, Kaposi's sarcoma, benign tumors such as hemangiomas, acoustic neuromas, neurofibromas, trachomas, and pyogenic granulomas, as well as other cancers which require neovascularization to support tumor growth. Other angiogenesis-dependent conditions include, for example, hereditary diseases such as Osler-Weber Rendu disease and haemorrhagic teleangiectasia; myocardial angiogenesis; plaque neovascularization; hemophiliac joints and wound granulation. Progression of tumors such as melanoma, from benign to metastatic disease, correlates with an increase in angiogenesis as well as an increase in expression of specific cell adhesion receptors including integrins (Srivastava, et al., Am. J. Pathol. 1988, 133:419-423; Koth, et al., N. Engl. J. Med. 1991, 325:171-182). Thus, angiogenesis likely plays a critical role in melanoma progression.

Examples of normal physiological processes involving angiogenesis include embryo implantation, embryogenesis and development, and wound healing. It is conceivable that angiogenesis can also be altered to beneficially influence normal physiological processes. Furthermore, studies have indicated that adipose tissue growth is dependent on angiogenesis, likely due to the need for recruitment of new blood vessels. Delivery of an angiogenesis inhibitor to mice was found to reduce diet-induced obesity, the most common type of obesity in humans (Brakenhielm, et al., Circ. Res. 2004, 94(12):1579-88). This finding suggests utility for angiogenesis inhibitors in addressing obesity and certain related conditions. Therefore, the inhibition of angiogenesis potentially can be applied in normal angiogenic responses where a prophylactic or therapeutic need or benefit exists.

The ECM, in a vastly simplified fashion, can be characterized as being composed of two general compartments. Embracing this two-compartment concept, the ECM can be divided into the interstitial ECM and the basal lamina or casement membrane. The basement membrane is a specialized form of ECM that separates both epithelia and endothelia from their underlying mesenchyme (Timpl, R. (1989) European Journal of Biochemistry 180(3), 487-502; Timpl, R., and Brown, J. C. (1996) Bioessays 18(2), 123-32; Yurchenco, P. D., and Schittny, J. C. (1990) FASEB Journal 4(6), 1577-90; Schittny, J. C., and Yurchenco, P. D. (1989) Current Opinion in Cell Biology 1(5): 983-8). Components of the basement membrane include, for example, laminin, Type IV collagen, enactin/nidogen, SPARC and perlecan, as well as other proteoglycans. These components exhibit a complex pattern of molecular interconnections and supramolecular assemblies that are organized into a mesh-like network.

The mesh-like network of the basement membrane is connected to the underlying interstitial matrix by a series of anchoring fibers including collagen-VII and fibrillin. Some of the well-characterized components include a variety of genetically distinct forms of collagen, such as collagen-I, II, III, and V. In addition, a number of non-collagenous glycoproteins also help compose the interstitial matrix including fibronectin, fibrinogen/fibrin, thrombospondin, and vitronectin (Adechi, E., Hopkinson, I., and Hayashi, T. (1997) International Review of Cytology 173, 73-156; Mosher, D. F., Sottile, J., Wu, C., and McDonald, J. A. (1992) Curr. Opion. Cell Biol. 4, 810-818). Finally, a number of proteoglycans also contribute to the complex architecture of the interstitial matrix. The networks of proteins that make up the ECM in conjunction with integrins function cooperatively to regulate new blood vessel development.

Historically, the ECM was thought to provide mechanical and structural support to cells and tissues. However, following the development of new molecular, cellular and biochemical techniques, this limited view of the ECM has expanded dramatically. In fact, the ECM can be defined in broad terms as a complex interconnected network of fibrous proteins, proteoglycans and structural glycoproteins that provide both mechanical and biochemical regulatory functions to cells and tissues. In angiogenesis, the regulatory information contained within the three dimensional structure of the ECM must be recognized and transferred to recipient cells capable of forming new blood vessels. To this end, integrin-mediated ligation of ECM components has been shown to activate distinct signal transduction pathways, which in turn may regulate neovascularization.

An important group of molecules that mediate cellular interactions with the ECM include the integrin family of cell adhesion receptors. Integrins are a family of heterodimeric cell surface proteins composed of non-covalently associated α and β chains (Jin, et al., Br. J. Cancer. 2004, 90:561-565; Bershadsky, et al., Annu. Rev. Cell Dev. Biol. 2003, 19:677-695, and; Parise, et al., Semin. Cancer Biol. 2003, 10:407-414). Integrins not only facilitate physical interactions with the ECM but also play critical roles in bi-directional signaling between the ECM and cells. In this regard, αvβ3 is one of the most well-studied integrins thought to play a critical role in invasive cellular processes such as angiogenesis and tumor invasion (Jin, et al., Br. J. Cancer. 2004, 90:561-565; Bershadsky, et al., Annu. Rev. Cell Dev. Biol. 2003, 19:677-695; Parise, et al., Semin. Cancer Biol. 2003, 10:407-414). In fact, expression of αvβ3 in endothelial cells regulates cell survival and apoptosis by a mechanism that likely depends on P53 (Stromblad, et al., J. Clin. Invest. 1996, 98:426-433; Stromblad, et al., J. Biol. Chem. 2002, 277:13371-13374; Lewis, et al., Proc. Natl. Acad. Sci. USA. 2002, 99:3627-3632). Therefore, αvβ3 ligation may suppress p53 activity. Furthermore, antagonists of αvβ3 failed to inhibit retinal neovascularization in p53 null mice. Stromblad, et al., J. Clin. Invest. 1996, 98:426-433; Stromblad, et al., J. Biol. Chem. 2002, 277:13371-13374).

Studies have indicated that αvβ3 plays a critical role in angiogenesis since antagonists directed to αvβ3 inhibit angiogenesis and tumor growth in multiple models (Brooks, et al., Science 1994, 264:569-571; Brooks, et al., Cell, 1994, 79:1157-1164; Brooks, et al., J. Clin. Invest. 1995, 96:1815-1822). In recent studies, mice lacking expression of αvβ3 exhibited enhanced growth of transplanted tumors (Taverna, et al., Proc. Natl. Acad. Sci. USA. 2001, 101:763-768). Thus, the molecular mechanisms by which αvβ3 regulates angiogenesis and tumor growth are complex and, to date, are not completely understood. Interestingly, αvβ3 and αvβ5 may regulate angiogenesis induced by distinct growth factors by mechanisms dependent on differential phosphorylation of Raf (Hood, et al., J. Cell Biol. 2003, 162:933-943; Alavi, et al., Science 2003, 301:204-206). Studies have also provided evidence that integrins can regulate signaling cascades in both the unligated and ligated states (Stupack, et al., J. Cell Biol. 2001, 155:459-470). Furthermore, studies suggest that unligated αvβ3 may lead to induction of apoptosis by a mechanism involving recruitment of caspase-8 (Stupack, et al., J. Cell Biol. 2001, 155:459-470). Thus, the ability of αvβ3 to either interact, or not, with distinct ligands may differentially impact invasive cellular behavior. However, gene modulation resulting from binding of integrins to cryptic epitopes of ECM components has not been characterized or systematically studied.

Proteolytic activity plays a crucial role in controlling angiogenesis by releasing matrix-sequestered growth factors as well as remodeling ECM proteins. While many ECM proteins have been shown to bind to αvβ3 in vitro, the physiological relevance of these interactions is not completely understood. ECM remodeling of the matrix can alter the three-dimensional structure of ECM proteins such as collagen and laminin, thereby exposing cryptic regulatory sites that are recognized by integrins including αvβ3 (Xu, et al., J. Cell Biol. 2001, 154:1069-1079; Hangai, et al., Am. J. Pathol. 2002, 161:1429-1437; Xu, et al., Hydridoma 2000, 19:375-385).

Other ligands including, but not limited to, fibrin, fibrinogen, laminin, thrombospondin, vitronectin, von Willebrand's factor, osteospontin and bone sialoprotein I also bind to αvβ3. The physiological importance of cellular interactions with these cryptic sites has been suggested, since function-blocking Mabs directed to the HUIV26 cryptic collagen site block angiogenesis and tumor growth in a number of animal models (Xu, et al., J. Cell Biol. 2001, 154:1069-1079; Hangai, et al., Am. J. Pathol. 2002, 161:1429-1437; Xu, et al., Hydridoma 2000, 19:375-385). The HUIV26 cryptic collagen epitope is recognized by αvβ3 integrin, which is highly expressed in tumor-associated blood vessels. Manipulating the interactions between αvβ3 and ECM components could provide a productive strategy for identifying methods to treat tumor development processes, including, but not limited to, tumor metastasis, tumor growth, angiogenesis, cell migration, cell adhesion and cell proliferation. However, the genes regulated in response to interactions involving integrin receptors and cryptic ECM components have not been previously characterized, and relatively little is known concerning the potential role of these interactions in tumor development processes.

Other proteins appear to be involved in integrin signaling and include, for example, Insulin Growth Factor Binding Proteins (IGFBPs). IGFBPs are a family of secreted proteins that function to regulate IGF-signaling by binding to IGFs, thereby disrupting IGF receptor binding and subsequent signaling (Pollak, et al., Nat. Rev. Cancer 2004, 4:505-518; Mohan, et al., J. Endocrinol. 2002, 175:19-31; LeRoith, et al., Cancer Lett. 2003, 195:127-137). Specific IGFBPs may directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). IGFBPs may regulate cellular adhesion, migration and tumor growth by both IGF-dependent and independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302). However, regulation of these cellular processes by integrin-receptor binding of IGFBPs, and the exact role of IGFBPs in these processes, have not been established.

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to metastasis and tumor growth. There is evidence for the importance of numerous molecular regulators that contribute to new blood vessel growth, including matrix-degrading proteases such as MMP-9, angiogenesis inhibitors such as TSP-1 and angiogenic growth factors such as VEGF (see, e.g., Yu, et al., Proc. Natl. Acad. Sci. USA 1999, 96:14517-14522; Dameron, et al., Science 1994, 265:1582-1584). These molecular regulators, the proteins that in turn regulate them, and a number of other molecules potentially affect angiogenesis and metastasis. However, the exact mechanisms of the regulation of these and related processes, including the genes and gene expression patterns involved, have not been determined.

Further, the protein Id-1 has been reported to repress TSP-1 expression and regulate angiogenesis in vivo (Volpert, et al., Cancer Cell 2002, 2(6):473-83). P53, a tumor-suppressor protein, has also been reported to play an important role in controlling expression of proteins known to regulate angiogenesis, including VEGF and thrombospondin-1 (TSP-1) (Yu, et al., Proc. Natl. Acad. Sci. USA 1999, 96:14517-14522 and Dameron, et al., Science 1994, 265:1582-1584). The p53 status of tumors is believed to impact the efficacy of anti-angiogenic, chemotherapeutic and radiation therapy for the treatment of malignant tumors (Yu, et al., Science 2002, 295:1526-1528; Martin, et al., Cancer Res. 1999, 59:1391-1399; Fridman, et al., Oncogene 2003, 22:9030-9040; Gudkov, et al., Nat. Rev. Cancer 2003, 3:117-128). Despite the possibility that these and other proteins are involved in the integrin-mediated regulation of tumor development processes, e.g., angiogenesis, metastasis, cell adhesion, cell migration, cell proliferation, tumor growth and the regulation of specific genes in response to αvβ3 binding of ECM component cryptic epitopes has not been previously characterized. This application identifies the connection between αvβ3 binding of ECM component cryptic epitopes and the regulation of genes involved in tumor development processes.

### SUMMARY OF THE INVENTION

Provided herein is a method of treating an angiogenesis-dependent condition in a mammal in need of such treatment, said method comprising administering to said mammal: a therapeutically-effective amount of a first antagonist that specifically binds to an extracellular matrix (ECM) component; and a therapeutically-effective amount of a second antagonist that specifically binds to an integrin; in a combination treatment regimen.

Also provided herein is a method of treating an angiogenesis-dependent condition in a mammal in need of such treatment, said method comprising administering to said mammal: (1) a therapeutically-effective amount of a first antagonist that specifically binds to an extracellular matrix (ECM) component; and (2) a therapeutically-effective amount of a second antagonist in a combination treatment regimen, wherein the second antagonist can be, for example, an antagonist that specifically binds to a second ECM component or an antagonist that specifically binds to the same ECM component as said first antagonist.

Provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to an integrin; in a combination treatment regimen, wherein said angiogenesis-dependent condition is an angiogenic disease.

Provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to an integrin; in a combination treatment regimen, wherein said angiogenesis-dependent condition is a cancer-associated disorder.

Provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to an integrin; in a combination treatment regimen, wherein said angiogenesis-dependent condition is a solid tumor.

Provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to a second ECM component; in a combination treatment regimen, wherein said angiogenesis-dependent condition is an angiogenic disease.

In other embodiments, provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to a second ECM component; in a combination treatment regimen, wherein said angiogenesis-dependent condition is a cancer-associated disorder.

In other embodiments, provided herein is a method of treating an angiogenesis-dependent condition in a mammal in need of such treatment, said method comprising administering to said mammal: a therapeutically-effective amount of a first antagonist that specifically binds to an extracellular matrix (ECM) component; a therapeutically-effective amount of a second antagonist that specifically binds to a second ECM component; in a combination treatment regimen, wherein said angiogenesis-dependent condition is a solid tumor.

In non-limiting embodiments, said ECM components of the methods are specifically bound by the antagonist and include, for example, cryptic collagen epitopes, cryptic laminin epitopes, fibronectin, vitronectin, fibrinogen, thrombospondin, osteopontin, tenascin and vWF.

The present application contemplates methods wherein said integrin is, for example, one or more of the integrins listed in Table II.

The present application contemplates methods wherein said integrin is, for example, one or more of the integrins listed in Table II.

The present application contemplates methods wherein said first and said second antagonist binds the same ECM component.

The present application contemplates methods wherein said method ECM component is denatured collagen. Alternatively, the present application contemplates methods wherein said method ECM component is denatured laminin.

The present application contemplates methods wherein said first and said second antagonist bind different or the same or multiple sites on the same ECM component.

The present application contemplates methods wherein the antagonists are, for example, antibodies, antibody fragments, peptides (linear or circular), a combination of antibodies and peptides, or a combination of antibody fragments and peptides. The antibody fragment can be, for example, a fragment such as a Fab, a F(ab′)₂, a Fv, a scFv, a Fd or a single chain binding polypeptide.

Any of the methods provided herein can include administering one or more cancer therapies, including, but not limited to, an antagonist that bind to the same or a different ECM component, an antagonist that binds to a different site on the same ECM component, an antagonist that binds to an integrin, one or more chemotherapeutic treatment, one or more biological therapy, one or more immunotherapy, one or more radiation therapy, one or more hormonal therapy, surgery, and a combination thereof.

Provided herein is a composition comprising a therapeutically effective amount of a first antagonist that specifically binds to an ECM component and therapeutically effective amount of a second antagonist that specifically binds to one of the following: different ECM components, a different site on the same ECM component or an integrin.

Provided herein is a composition comprising a therapeutically effective amount of a first antagonist that specifically binds to an integrin and a therapeutically effective amount of a second antagonist that specifically binds to one of the following: a different integrin, a different site on the same integrin, a different component of the same integrin, or a different site on the same component of the same integrin.

In the methods provided herein, the first antagonist and the second antagonist can be administered in combination sequentially or concurrently.

Provided herein is a method of preventing, treating or managing cancer in a patient in need thereof, said method comprising administering to said patient a dose of an effective amount of an ECM-component antagonist or an antagonist that competes with the joining of said ECM-component antagonist for binding to the ECM-component, and a dose of an effective amount of one or more other cancer therapies.

In a non-limiting embodiment, the ECM-component antagonist can be, for example, an ECM-component-binding antibody or an antigen-binding fragment thereof, or an antibody or an antigen-binding fragment thereof that competes with said ECM-component-binding antibody for binding to the ECM-component.

In one non-limiting embodiment, the ECM component recognized by said ECM-component-binding antagonist is, for example, a cryptic collagen epitope, a cryptic laminin epitope, fibronectin, vitronectin, fibrinogen thrombospondin, osteopontin, tenascin or vWF.

The antagonists of the compositions and methods provided herein can be, for example, a peptide (linear or circular), an antibody, an antibody fragment, a combination of an antibody and peptide, or a combination of an antibody fragment and a peptide. The antibody fragment can be, for example, a fragment such as a Fab, a F(ab′)₂, a Fv, a scFv, a Fd or a single chain binding polypeptide that functions to bind a site (epitope) on an antigen.

Provided herein are methods wherein said ECM-component antagonist or an antagonist that competes with said ECM-component-binding antagonist for binding to the ECM-component, is administered to said patient sequentially or concurrently with the administration of one or more other cancer therapies.

In methods provided herein wherein one or more cancer therapies are administered, the cancer therapy can be one or more of the following: an antagonist that binds to a second ECM component, an antagonist that binds to an integrin, one or more chemotherapeutic agent, one or more biological therapies, one or more immunotherapies, one or more radiation therapies, one or more hormonal therapies, surgery or a combination thereof.

In one non-limiting embodiment, the ECM component binding antagonist is an integrin.

The present invention contemplates methods wherein said cancer is, for example, a cancer of the head neck, eye, mouth, throat, esophagus, chest, bone, lung, colon, rectum, stomach, prostate, breast, ovary, testes, thyroid, blood, kidney, liver, pancreas or brain or central nervous system.

In any of the methods provided herein, the patient or mammal to be treated can be, for example, a human.

Provided herein is a composition of a therapeutically effective amount of one or more ECM-component antagonists or an antagonist that competes with the binding of an ECM component antagonist to an ECM component and a therapeutically effective amount of one or more cancer therapies.

In one non-limiting embodiment, the compositions for the one or more cancer therapies can be, for example, an ECM-component antagonist or an antagonist that competes with the binding of an ECM component antagonist to an ECM component. The present invention also includes compositions wherein said ECM-component antagonists are antagonists of the same ECM component or antagonists of different ECM components. If the antagonists are antagonists of the same ECM component, the antagonists can bind the same or different sites on the same ECM component.

### BRIEF DESCRIPTION OF THE DRAWINGS

FIG. 1 Expression of αvβ3 Enhanced Human Melanoma Growth In vivo. Human melanoma cells expressing (M21) or lacking αvβ3 (M21L) were injected (1×10⁶) subcutaneously into nude mice. Tumor cells were allowed to grow for 7 days. Tumor volumes were calculated using the formula V=(L²×W)/2 where V=volume, L=length and W=width. Data bars represent mean tumor volumes±standard errors from 5 animals per condition. αvβ3-expressing M21 cells formed tumors that were approximately 9-fold larger (P<0.05) than tumors from cells that lacked αvβ3 (M21L). Experiments were completed 3 times with similar results.

FIG. 2 Isolation of αvβ3 Expression Variants of Human ECV Bladder Carcinoma (Parental Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C10) or control (non-specific Ab) in parental ECV carcinoma cells. As shown, the parent ECV carcinoma cells expressed high surface levels of αvβ3 (middle panel; FIG. 2B) and β1 integrins (bottom panel; FIG. 2C) compared to controls (upper panel; FIG. 2A).

FIG. 3 Isolation of αvβ3 Expression Variants of Human ECV Bladder Carcinoma (Variant Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C10) or control (non-specific Ab; upper panel; FIG. 3A) in negative selected carcinoma cells (ECVL). Negatively-selected (ECVL) cells expressed no detectable αvβ3 on the cell surface (middle panel; FIG. 3B). Reduction of αvβ3 expression in these cells resulted in little if any change in β1 integrin expression (bottom panel; FIG. 3C).

FIG. 4 Expression of αvβ3 Enhanced Human Carcinoma Growth In vivo. Human carcinoma cells expressing (ECV) or lacking αvβ3 (ECVL) were injected (1×10⁶) subcutaneously into nude mice. Tumor cell variants were allowed to grow for 14 days. Tumor volumes were calculated using the formula V=(L²×W)/2 where V=volume, L=length and W=width. Data bars represent mean tumor volumes±standard errors from 5 animals per condition. αvβ3-expressing ECV cells formed tumors that were approximately 3-fold larger than ECVL cells lacking αvβ3. Experiments were completed 3 times with similar results.

FIG. 5 Expression of αvβ3 Does Not Enhance Human Carcinoma Growth In vitro. Human carcinoma cells expressing (ECV) or lacking αvβ3 (ECVL) were allowed to proliferate in vitro. Tumor cells (ECV and ECVL) were seeded into microtiter plates and allowed to proliferate in low serum (1.0%) containing medium over a time course of 3 days. Proliferation was quantified by monitoring mitochondrial dehydrogenase activity at 490 mn. Data bars represent mean O.D.± standard deviation from triplicate wells. Little if any change in proliferation was detected between ECV and ECVL cells in vitro. Experiments were completed 3 times with similar results.

FIG. 6 Reduced Angiogenesis in Tumors Lacking Integrin αvβ3. Tumor angiogenesis was quantified in tumors expressing (M21 and ECV) or lacking αvβ3 (M21L and ECVL) by microvascular density counts. Frozen sections of tumors were stained with an anti-CD31 polyclonal antibody. The number of CD-31 positive blood vessels was counted per 200× microscopic fields (FIG. 6A). Quantification of tumor angiogenesis in M21 melanoma tumor variants (FIG. 6B). Quantification of tumor angiogenesis in ECV carcinoma variants. Data bars represent the mean blood vessel counts per 200× field (N=10 fields per specimen with 3 specimens per tumor type). The αvβ3-expressing tumors (M21 and ECV) exhibited a significant (P<0.05) 2.0- to 2.5-fold increase in the number of blood vessels compared to tumors lacking avβ3 (M21L and ECVL).

FIG. 7 Enhanced Blood Flow in αvβ3-Expressing Tumors. Melanoma tumors expressing (CS1β3) and lacking (CS1) αvβ3 were scanned using a Moor LDI VR laser Doppler. Laser Doppler scans on tumor and tissue 0.5 cm surrounding the tumor were performed. The tumor and surrounding tissue were scanned in a raster pattern and the Doppler shifts within the microvasculature were measured. The figure shows representative scans of flow. CS1β3 tumors were associated with elevated levels of blood flow as compared to CS1 tumors.

FIG. 8 Quantification of Enhanced Blood Flow in αvβ3 Expressing Tumors. Melanoma tumors expressing (CS1β3) and lacking (CS1) αvβ3 were scanned using a Moor LDI VR laser Doppler. Laser Doppler scans on tumor and tissue 0.5 cm surrounding the tumor were performed. The tumor and surrounding tissue were scanned in a raster pattern and the Doppler shifts within the microvasculature were measured. The figure shows quantification of tumor-associated blood flow reported numerically using Moor LDI Imaging Software, v3.09. N=5. CS1β3 tumors were associated with an approximately 40% increase in blood flow compared to CS1 tumors (P<0.05) that lacked αvβ3.

FIG. 9 Conditioned Medium (CM) from Tumors Cells Lacking αvβ3 Inhibits Angiogenesis. Filter disc-containing bFGF (12 ng) were place on the CAMs of 10-day old chick embryos. Twenty-four hours later, the embryos were treated topically with serum free CM (40 μl). At the end of 3 days, angiogenesis was quantified by counting blood vessel branch points. The figure shows the effects of CM from ECV cells on bFGF-induced angiogenesis. Data bars represent the mean number of blood vessel branch point±standard deviation from 8 to 10 embryos per condition. CM from ECVL cells significantly (P<0.001) inhibited bFGF-induced angiogenesis by greater than 90% as compared to control. CM from ECV cells had no significant effect (P>0.300) on angiogenesis. Experiments were completed twice with similar results.

FIG. 10 Conditioned Medium (CM) from Tumors Cells Lacking αvβ3 Inhibit Endothelial Cell Proliferation. Endothelial cells (HUVECs) were seeded into microtiter plates in the presence or absence of serum-free CM (25 μl) from either ECV or ECVL and allowed to proliferate in low serum (5.0%) medium for 24 hours. Proliferation was quantified by monitoring mitochondrial dehydrogenase activity at 490 nm using the WST-1 proliferation kit (Chemicon). Data bars represent mean O.D.±standard deviation from triplicate wells. CM from ECVL cells inhibited HUVEC cell proliferation by approximately 50%, while CM from ECV cells had no effect. Experiments were completed 3 times with similar results.

FIG. 11 Conditioned Medium (CM) from Tumor Cells (ECVL) Lacking αvβ3 Inhibits Tumor Growth In vivo. Tumor cells (CS1) were seeded on the CAMs of 10-day old chick embryos. Twenty-four hours later, the embryos were treated daily by topical addition of serum-free CM (25 μl) from ECVL. Tumors were allowed to grow for 7 days, then harvested and wet weights determined. Data bars represent the mean tumor weights±standard deviation from 8 to 10 embryos per condition. The control was serum-free concentrated medium only. Daily treatments with CM from ECVL tumor cells resulted in a significant decrease (P<0.05) in tumor weight by approximately 50% compared to controls. Experiments were completed twice with similar results.

FIG. 12 Conditioned Medium (CM) from Tumor Cells (M21) Lacking αvβ3 Inhibits Tumor Growth In vivo. Tumor cells (CS1) were seeded on the CAMs of 10-day-old chick embryos. Twenty-four hours later, the embryos were treated daily by topical addition of serum-free CM (25 μl) from M2 μL tumor cells. Tumors were allowed to grow for 7 days, then harvested and wet weights determined. Data bars represent the mean tumor weights±standard deviation from 8 to 10 embryos per condition. The control was serum-free concentrated medium only. Daily treatments with CM from M21 tumor cells resulted in a significant decrease (P<0.05) in tumor weight by approximately 50% compared to controls. Experiments were completed twice with similar results.

FIG. 13 Elevated Levels of TSP-1 in CM from Tumor Cells Lacking αvβ3 (ECVL). Concentrated serum-free CM was examined for the relative levels of TSP-1 by ELISA. CM (25 μl) from ECV tumor cells was diluted in coating buffer 1:1 and incubated in microtiter wells for 18 hours at 4° C. The wells were washed, blocked and incubated with anti-TSP-1 Mab or control non-specific antibody. The relative levels of TSP-1 were detected by incubation with HRP-labeled goat anti-mouse antibody. All data were corrected for non-specific binding. Data bars represent the mean O.D.±standard deviations from triplicate wells. The relative levels of TSP-1 were increased in CM from ECVL nearly 4-fold compared to CM from ECV. Experiments were completed 3 times with similar results.

FIG. 14 Elevated Levels of TSP-1 in CM from Tumor Cells Lacking αvβ3 (M21L). Concentrated serum-free CM was examined for the relative levels of TSP-1 by ELISA. CM (25 μl) from M21 tumor cells was diluted in coating buffer 1:1 and incubated in microtiter wells for 18 hours at 4° C. The wells were washed, blocked and incubated with anti-TSP-1 Mab or control non-specific antibody. The relative levels of TSP-1 were detected by incubation with HRP-labeled goat anti-mouse antibody. All data was corrected for non-specific binding. Data bars represent the mean O.D.±standard deviations from triplicate wells. The relative levels of TSP-1 were increased in CM from M21L nearly 2-fold compared to CM from M21. Experiments were completed 3 times with similar results.

FIG. 15 TSP-1-Depleted ECVL CM Fails to Inhibit Endothelial Cell Proliferation. Endothelial cells (HUVECs) were seeded into microtiter plates in the presence or absence of TSP-1 depleted CM or non-specific antibody depleted CM (25 μl) from ECVL cells and allowed to proliferate in low serum (5.0%) containing medium for 24 hours. Proliferation was quantified by monitoring mitochondrial dehydrogenase activity at 490 nm using the WST-1 proliferation kit. Data bars represent mean O.D.±standard deviation from triplicate wells. Control-depleted ECVL conditioned medium inhibited HUVEC proliferation by approximately 50% compared to controls (i.e., no treatment). In contrast, CM from ECVL cells that was depleted of TSP-1 exhibited little if any effects on HUVEC cell proliferation. Experiments were completed 2 times with similar results.

FIG. 16 Elevated Levels of IGFBP-4 in Tumor Cells Following siRNA-Mediated Reduction in β3 Integrin. Expression of β3 integrin within M21 and ECV cells was reduced by siRNA. FIG. 16A shows Western blot analysis of β3 integrin or control protein β-Actin in M21 cells transfected with either β3-specific and control scrambled siRNA. β3 integrin was reduced by greater than 70% in β3 siRNA transfected cells as compared to controls, while no change in β-Actin was observed. FIG. 16B shows Western blot analysis of IGFBP-4 or control protein β3-Actin in M21 cells transfected with either β3-specific or a control scrambled siRNA. Expression of IGFBP-4 was increased (>60%) in β3 siRNA transfected cells as compared to control cells.

FIG. 17 Elevated Levels of TSP-1 in Tumor Cells Following siRNA-Mediated Reduction in β3 Integrin. Expression of β3 integrin within M21 and ECV cells was reduced by siRNA. The figure shows real time PCR analysis of TSP-1 expression in ECV cells transfected with either β3 specific or a control scrambled siRNA. The relative levels of TSP-1 were significantly elevated in β3 siRNA transfected ECV cells in which β3 integrin is significantly reduced as compared to control transfected cells.

FIG. 18 TSP-1 Expression Following αvβ3-Integrin-Specific Ligation. Culture plates were coated with either αvβ3 specific ligands (Vitronectin and anti-αvβ3 Mab LM609) or P1 integrin ligands (triple helical collagen type-IV and anti-α1 specific Mab P4C10). M21 cells were allowed to interact with specific ECM proteins. The relative levels of TSP-1 were examined by real time PCR following normalization to non-specific ligand (poly-L lysine). CM from M21 cells interacting with the non-αvβ3 ECM ligand collagen type-IV resulted in an approximately 4-fold increase in TSP-1 as compared to CM from cells interacting with the known αvβ3 ligand vitronectin.

FIG. 19 Suppression of TSP-1 Expression Following αvβ3 Integrin Specific Ligation. Culture plates were coated with either αvβ3 specific ligands (vitronectin and anti-αvβ3 Mab LM609) or β1 integrin ligands (triple helical collagen type-IV and anti-β1 specific Mab P4C10). M21 cells were allowed to interact with specific anti-integrin Mabs. The relative levels of TSP-1 were examined by real time PCR following normalization to non-specific ligand (poly-L lysine). The relative levels of TSP-1 in cells ligating αvβ3 was reduced by greater than 50% compared to cells ligating β1 integrins as measured by real time PCR.

FIG. 20 Inhibition of αvβ3-Mediated Ligation Increases TSP-1 Expression in M21 Cells. M21 cells were seeded on denatured collagen type-IV coated plates in the presence or absence of anti-αvβ3 specific Mab LM609 or an isotype-matched control antibody. Cells were allowed to incubate for 12 hours in 1.0% serum-containing medium. Expression of TSP-1 was examined by real time PCR. Expression levels were normalized for β2 macroglobulin (B2M). The relative level of TSP-1 RNA was elevated by approximately 8-fold in M21 cells treated with the anti-αvβ3 specific Mab LM609 compared to an isotype-matched control antibody as measured by real time PCR

FIG. 21 Inhibition of αvβ3-Mediated Ligation Increases IGFBP-4 Expression in M21 Cells. M21 cells were seeded on denatured collagen type-IV coated plates in the presence or absence of anti-αvβ3 specific Mab LM609 or an isotype matched control antibody. Cells were allowed to incubate for 12 hours in 1.0% serum containing medium. Expression of IGFBP-4 was examined by RT-PCR. Expression levels were normalized for β2 macroglobulin (B2M). The relative level of IGFBP-4 was elevated by approximately 8-fold in M21 cells treated with the anti-αvβ3 specific Mab LM609 as compared to an isotype matched control antibody.

FIG. 22 Inhibition of αvβ3-Mediated Ligation Increases IGFBP-4 RNA Expression in M21 Tumors. M21 cells were seeded on denatured collagen type-IV coated plates in the presence or absence of anti-αvβ3 specific Mab LM609 or an isotype-matched control antibody. Cells were allowed to incubate for 12 hours in 1.0% serum-containing medium. The figure shows expression of IGFBP-4 in M21 tumors grown in chick embryo, either untreated (NT; FIG. 22A) or treated systemically with Mab LM609 (FIG. 22B) or control non-specific antibody (FIG. 22C; 100 μg/embryo) N=5. Expression of IGFBP-4 was significantly enhanced in M21 tumors grown in the chick embryo following treatment with Mab LM609.

FIG. 23 Elevated Levels of IGFBP-4 Protein in CM from Tumor Cells Lacking αvβ3. Conditioned Medium (CM) was evaluated for the relative levels of IGFBP-4 by ELISA. The figure shows data obtained using CM (25 μl), from ECV and ECVL tumor cells, diluted in coating buffer 1:1 and incubated in microtiter wells. The wells were washed, blocked and incubated with anti-IGFP-3 and IGFBP-4 Mabs. The relative levels of IGFBP-3 and IGFBP-4 were detected by incubation with HRP-labeled goat anti-mouse antibody. All data were corrected for non-specific binding against a control non-specific antibody. Data bars represent the mean O.D.±standard deviations from triplicate wells. The relative levels of IGFBP-4 increased in CM from ECVL by greater than 10-fold compared to ECV while little, if any, change in the levels of IGFBP-3 was observed. Experiments were completed 3 times with similar results.

FIG. 24 Elevated Levels of IGFBP-4 Protein in CM from Tumor Cells Lacking αvβ₃ as Determined by Western Blotting. CM was examined for the relative levels of IGFBP-4 by Western blot. The figure shows analysis of CM from ECV and ECVL cells, for IGFBP-4, or using soluble fibronectin as control. IGFBP-4 was dramatically increased in the CM of ECVL cells as compared to ECV cells while little or no change was detected in soluble fibronectin.

FIG. 25 Effect of mAb HUIV26 in Combination with CLK-Peptide on Adhesion of M21 Cells to Denatured Collagen Type IV. Microtiter wells were coated with either native or denatured collagen type-IV. M21 cells were allowed to bind to the coated substrates in the presence of CLK-peptide, mAb HUIV26, or both. The combination of both CLK-peptide and mAb HUIV26 had a greater effect on adhesion than did either antagonist alone.

FIG. 26 Binding of CLK-Peptide and Mab HUIV26 to Denatured Collagen Type IV. To examine the epitope bound by CLK and HUIV26, competition ELISAs were carried out. Microtiter wells were coated with denatured collagen type-IV (10 μg/ml), and mAb HUIV26 was allowed to bind the denatured collagen in the presence or absence of CLK-peptide. As shown in FIG. 26A, CLK-peptide did not block binding of mAb HUIV26. FIG. 26B shows that mAb HUIV26 did not block binding of CLK-peptide.

FIG. 27 Inhibition of Binding of Labeled CLK-Peptide to Denatured Collagen Type-IV by mAb HUI77. To examine the binding specificity of the synthetic CLK-peptide, the peptide was labeled and solid phase ELISAs were performed. Denatured collagen type-IV (10 μg/ml) was coated on microtiter wells and binding of either HRP-labeled CLK-peptide or mAbs (mAbs HU177 or HUIV26) was measured by ELISA at a wavelength of 490 mL As shown in the figure, Mab HUI77, but not HUIV26, specifically inhibited CLK-binding to denatured collagen type-IV.

FIG. 28 Inhibition of Binding of ⁹⁹Tc-Labeled CLK-Peptide to Denatured Collagen Type-IV by mAb HUI77. Denatured collagen type-IV (10 μg/ml) was coated on microtiter wells and binding of ⁹⁹Tc-labeled CLK-peptide in the presence or absence of unlabeled CLK-peptide or mAb HUI77 was measured. As shown in the figure, ⁹⁹Tc-CLK-peptide (10 μg/ml, 50 μl/well) specifically binds to denatured collagen type-IV (first bar from left). Unlabeled CLK-peptide exhibited a dose dependent inhibition of 99Tc-CLK-peptide binding (second and third bars from left). ⁹⁹Tc-CLK-peptide was also allowed to bind to denatured collagen type-IV in the presence or absence of Mab HU177 (fourth and fifth bars from left). Mab HUI77 also inhibited ⁹⁹Tc-labeled CLK-peptide. These findings provide further evidence that the CLK-peptide binds to a similar, if not identical, epitope recognized by the mAb HUI77.

FIG. 29 CDRs of Humanized Antibodies Derived from HUI77 and HUIV26. The diagram shows comparisons among five denatured-collagen-binding antibodies having CDRs (underlined amino acid sequences) derived from the CDR sequences of murine monoclonal antibody HUI77 (shown in the first and second numbered lines of sequence), or from HUIV26 (shown in the second and third numbered lines of sequence).

Variable Heavy Chains Derived from HUI77

| | |
|---|---|
| HUI77 | SEQ ID NO: 1 |
| Human DP-28 | SEQ ID NO: 2 |
| 12F10Q | SEQ ID NO: 3 |
| QH2b-B7 | SEQ ID NO: 4 |
| Qcom1D3 | SEQ ID NO: 5 |
| QhuD9 | SEQ ID NO: 6 |
| QhuD93 | SEQ ID NO: 7 |

Variable Light Chains Derived from HUI77

| | |
|---|---|
| HUI77 | SEQ ID NO: 8 |
| Human DPK13 | SEQ ID NO: 9 |
| 12F10Q | SEQ ID NO: 10 |
| QH2b-B7 | SEQ ID NO: 11 |
| Qcom1D3 | SEQ ID NO: 12 |
| QhuD9 | SEQ ID NO: 13 |
| QhuD93 | SEQ ID NO: 14 |

Variable Heavy Chains Derived from HUIV26

| | |
|---|---|
| HUIV26 | SEQ ID NO: 15 |
| Human VH3-11 | SEQ ID NO: 16 |
| 2D4 | SEQ ID NO: 17 |
| 2D4H1-C3 | SEQ ID NO: 18 |
| DcomD7 | SEQ ID NO: 19 |
| DhuG5 | SEQ ID NO: 20 |
| DhuH8 | SEQ ID NO: 21 |

Variable Light Chains Derived from HUIV26

| | |
|---|---|
| HUIV26 | SEQ ID NO: 22 |
| Human DPK24 | SEQ ID NO: 23 |
| 2D4 | SEQ ID NO: 24 |
| 2D4H1-C3 | SEQ ID NO: 25 |
| DcomD7 | SEQ ID NO: 26 |
| DhuG5 | SEQ ID NO: 27 |
| DhuH8 | SEQ ID NO: 28 |

The first continuous numbered line of sequence, labeled “Hu-I77 VH,” shows the amino acid sequence of the heavy chain variable region of mouse monoclonal antibody HUI77 (SEQ ID NO: 1). To construct the humanized antibodies, the HUI77 sequence was compared with human antibody sequences and the human antibody having the greatest similarity to the HUI77 sequence was selected as the human framework donor.

For the HUI77 heavy chain variable (V_{H}) region, human antibody DP-28 (SEQ ID NO: 2) was selected and changed according to methods known in the art so as to introduce the three mouse CDR sequences. The differences between the amino acid sequence of DP-28 and that of HUI77 are indicated in the line labeled “DP-28.” Five partially and fully humanized antibody V_{H} region sequences are illustrated. The partially humanized antibodies, 12F10Q (SEQ ID NO: 3), QH2b-B7 (SEQ ID NO: 4), and Qcom1D3 (SEQ ID NO: 5), contain up to three changes in the V_{H} region relative to DP-28. These three changes are shown in bold lettering outside the underlined CDRs, at positions 75, 79, and 85. The fully-humanized antibody QhuD93 (also referred to herein as “D93;” SEQ ID NO: 7)) contains no changes in the V_{H} region relative to DP-28. The three CDRs in the V_{H} region, as indicated by underlining, are located from 31 to 35b (heavy chain CDR1), 50 to 65 (heavy chain CDR2) and 95 to 102 (heavy chain CDR3).

With regard to the HU177 light chain variable (V_{L}) region, human antibody DPK13 (SEQ ID NO: 9) was selected and changed to introduce the three mouse light chain CDR sequences. The partially-humanized antibodies contain one change or fewer in the V_{L} region relative to DPK13. This change is shown in bold lettering outside the underlined CDRs, at position 15. The three CDRs in the V_{L} region, as indicated by underlining, are located from 24 to 34 (light chain CDR1), 50 to 56 (light chain CDR2) and 89 to 97 (light chain CDR3).

The amino acids in antibodies DP-28 (SEQ ID NO: 2), DPK13 (SEQ ID NO: 9), 12F10Q (SEQ ID NOS: 3 and 10), QH2b-B7 (SEQ ID NOS: 4 and 11), Qcom1D3 (SEQ ID NOS: 5 and 12), QhuD9 (SEQ ID NOS: 6 and 13) and QhuD93 (SEQ ID NOS: 7 and 14) that differ from the original murine variable region sequences, are shown in plain text outside the underlined CDRs.

According to the diagram, humanized variant QhuD93 has two differences in the heavy chain CDR1 from the heavy chain CDR1 of HUI77 (an S to P at position 32 and a G to W at position 35b), one change in the heavy chain CDR2 (a Y to T at position 59) and one change in the heavy chain CDR3 (an M to Q at position 100e). D93 has two differences in the light chain CDRs, one at position 27d and one at position 28 in light chain CDR1. These changes are all shown in bold lettering within the underlined CDRs.

The HUIV26 (SEQ ID NO: 25)-derived antibodies are similarly represented. The three CDRs in the VH region of this antibody are underlined and extend from residues 31 to 35 (heavy chain CDR1), 50 to 65 (heavy chain CDR2), and 95 to 102 (heavy chain CDR3). The three CDRs in the VL region are underlined and are located from residues 24 to 34 (light chain CDR1,), 50 to 56 (light chain CDR2), and 89 to 97 (light chain CDR3).

The fully-humanized antibody based on HUIV26, DhuH8 (also referred to herein as “H8”) has one change in heavy chain CDR1 (an S to T at position 34), two changes in heavy chain CDR2 (an I to A at position 57 and an S to Y at position 62), one change in heavy chain CDR3 (a Y to P at position 102), two changes in light chain CDR1 (an S to W at position 27e and a G to Y at position 27f), and one change in light chain CDR3 (an S to Q at position 93).

The asterisks shown in place of one-letter amino acid codes in the V_{H}3-11 light chain CDR3 indicate that with regard to this antibody family, the sequence at these positions varies among family members.

### DETAILED DESCRIPTION OF THE INVENTION

In describing the embodiments provided herein, the following terms will be employed, and are intended to be defined as indicated below. Unless otherwise indicated, all terms used herein have the same ordinary meaning as they would to one skilled in the art of the present application.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### Definitions

Combination Therapies

The present application contemplates treatments comprising combinations of agents and/or other cancer therapies, said combinations having an effect on angiogenesis and being useful in methods for treating angiogenesis-dependent conditions. Treatments as provided herein, for example, block interactions between two proteins by binding to one protein, binding to the other protein, or both. For example, agents can be used in combination to block integrin binding to an ECM component by interacting with the integrin, the ECM component, or both. Combinations of antagonists that bind to or interfere with interactions between different proteins, combinations of antagonists that bind to multiple parts of the same protein, and combinations of antagonists that bind to multiple proteins or protein binding sites are also contemplated. Further contemplated are treatments including bi- or multi-specific antagonists that interfere with more than one protein or protein-binding site.

Examples of potential targets of the combination treatments are described in Tables I through XVI. These tables are intended to provide examples rather than to be limiting. The potential protein targets and agents indicated in the tables, as well as many others, have been described in the literature. Also provided are lists of other cancer therapies that can be used in any of the combinations described herein.

In preferred embodiments, the proteins and agents listed in Tables I through VIII are used in combination with one or more denatured collagen antagonists as described herein. In other preferred embodiments, the protein targets and agents listed in Tables IX through XVI are used in combination with one or more denatured laminin antagonists as described herein. In other embodiments, one or more denatured collagen antagonists or one or more of the types of target proteins or agents listed in each of the tables can be used together in particular combinations.

Other useful therapeutic combinations include combinations of one or more of the denatured collagen antagonists selected from Table A. For example, useful combinations include HU1U26 and CLK Peptide, HU177 and HU1U26, D93 and CLK Peptide, and the remaining potential combinations that can be created from combining the antagonists of Table A.

Other useful therapeutic combinations include combinations with Vitaxin with the denatured collagen antagonists (Table A), denatured laminin antagonists (Table B), antibodies that bind integrins (Table II), antibodies that bind cell surface receptors (Table III), antibodies that bind laminin (Table IV), laminin peptides (Table IV), antibodies that bind ECM components (Table V), enzymes inhibitors (Table VI), immune stimulants (Table VII), tyrosine kinase receptor inhibitors (Table VIII).

Other useful therapeutic combinations include combinations comprising 2, 3, 4, 5, 6 or more individual components selected from (Tables A, B, and 1-15).

For example, an antagonist of denatured collagen type-IV (e.g., the monoclonal antibody HUI77, D93, or the CLK-peptide or another antagonist that competes with an antagonist of denatured collagen type-IV for binding to denatured collagen type IV) can be used with an antagonist of αvβ3 integrin (e.g., the monoclonal antibody Vitaxin or an antibody that competes with Vitaxin for binding to αvβ3 integrin). As discussed herein, αvβ3 integrin interacts with denatured collagen type-IV. The interaction between αvβ3 integrin and denatured collagen type-IV is disrupted by mAb HUI77. Therefore, a combination treatment including antibody HUI77 or D93 targeting αvβ3 integrin, and the mAb Vitaxin targeting αvβ3 integrin, would potentially inhibit distinct integrin-ECM component interactions.

Alternatively, for example, a combination could include an antagonist of αvβ3 integrin and mAb HUIV26. This combination therapy would potentially inhibit both participants in the interaction between αvβ3 integrin and its binding site on denatured collagen.

With regard to denatured laminin, the combination of an antagonist of αvβ3 integrin (e.g., Vitaxin or LM609 antibody) and an antibody specific for denatured laminin could more successfully disrupt the interaction between αvβ3 and its cryptic laminin binding site than either agent alone.

It will be apparent to one of skill in the art that other such targets are being identified, and antagonists to these targets are being developed, and that any combinations of antagonists to such targets with one another or with the antagonists listed herein, and/or other cancer therapies can be used in combinations according to any of the methods provided herein.

Provided herein is a composition of a pharmaceutically acceptable carrier/excipient and any of the combinations provided herein. The composition can further include a therapeutic moiety, an imaging or diagnostic moiety. When the composition is to be administered to a patient, the composition can be substantially free of pyrogens. Compositions include, for example, pharmaceutical compositions for the therapeutic and diagnostic methods described herein.

Combination of Anti-Denatured Collagen Antibodies, Peptides, and Antibody Binding Epitopes with Other Agents for Therapeutic or Diagnostic Use.

The present application provides a combination of antagonists of denatured or proteolyzed collagens, such as but not restricted to antibodies HUI77, HUIV26, XL313, QH2B, 2D4, H8 and D93, and CLK peptide, with other agents that potentially enhance the prophylactic or therapeutic profile of a single agent. The combination therapy may provide a synergistic and/or beneficial effect or may allow lower doses of a combination to provide a greater margin of safety. Encompassed herein are treatment protocols that enhance the prophylactic or therapeutic effect of an antagonist of denatured or proteolyzed collagens using a specific antibody, or a fragment derived from an antibody, compound cross-linked or genetically fused to an antibody, peptide or mimetic epitope which binds the antibody, an antibody gene(s) using gene therapies for preventing, managing, treating or ablation of cancer or other diseases.

TABLE A
Denatured Collagen Antagonists

| | |
|---|---|
| HUI77 | 2D4 |
| HU1V26 | H8 |
| XL313 | D93 |
| QH2B | CLK Peptide |

Therapeutic Combinations Comprising Denatured Collagen Antagonist and Collagen Derived Peptide

The present application describes combination of anti-denatured collagen antibodies or binding peptides (shown in Table A) with peptides derived from collagen peptide sequences shown in Table 1. Certain collagen peptides are referenced with names for potential therapeutic and/or diagnostic use, such as Arrestin, Canstatin, etc. For most therapeutic indications the compounds are administered to inhibit diseases such as cancer and inflammation but may be used to augment host biological responses in certain disease conditions. The collagen peptides can be delivered either by the same route of administration in combination with the anti-denatured collagen mAbs or via different routes of administration depending on the optimum formulation, concentration and bioactive species for a patient with a specific disease. One or more of the peptides may also be used in combination with the anti-denatured collagen mAbs and formulated either together or separately and delivered by the same or a different route of administration depending on the optimum formulation and bioactive species for a patient with a specific disease. The collagen peptides may also be covalently linked to the anti-denatured collagen mAbs to achieve a beneficial therapeutic effect certain combinations of anti-denatured collagen peptides and collagen peptides, either separate or cross-linked, can also be used in diagnostic assays.

TABLE I
Collagen-Derived Peptides

| Collagen Peptide | Parent Protein | Reference |
|---|---|---|
| Human Endostatin | NC1 domain, α1 chain of type XVIII | O'Reilly, MS et al. |
| | collagen | Cell 88, 277-285 |
| | | (1997). |
| Human Endostatin- | NC10 domain, α1 chain of type XV | Ramchandran, R et |
| like protein | collagen | al, Biochem. |
| | | Biophys. Res. |
| | | Comm 255, 735-739 (1999) |
| Arrestin | NC1 domain, α1 chain of type IV | Kalluri et al. |
| | collagen | Cold Spring Harb |
| | | Symp Quant Biol. |
| | | 2002; 67: 255-66 |
| Canstatin | NC1 domain, α2 chain of type IV | Kalluri et al. |
| Angiocol | collagen | Cold Spring Harb |
| | | Symp Quant Biol. |
| | | 2002; 67: 255-66; also |
| | | Biostratum Patent |
| Tumastatin | NC1 domain, α3 chain of type IV | Kalluri et al. |
| | collagen | Cold Spring Harb |
| | | Symp Quant Biol. |
| | | 2002; 67: 255-66 |
| α6(IV) NC1 domain | NC1 domain, α6 chain of type IV | Kalluri et al. |
| | collagen | Cold Spring Harb |
| | | Symp Quant Biol. |
| | | 2002; 67: 255-66 |
| Denatured Collagen | Peptides bound by mAbs, HUI77, | — |
| Derived Peptides | HUIV26, D93, H8 and antibodies or | |
| | fragments that compete with binding | |
| | to HUI77, HUIV26, D93 and H8 to | |
| | their respective binding sites | |

Therapeutic Combinations Comprising Denatured Collagen Antagonists and Integrin Peptides, Antibodies and Derivatives

The present application describes combinations of anti-denatured collagen antibodies/peptides (shown in Table A) with peptides, antibodies, and siRNA, DNA or RNA sequences that encode integrin peptide sequences including those shown in Table II. Some the integrin peptides are referenced with names for potential therapeutic and/or diagnostic use, such as Tysabri, Vitaxin and ReoPro. For most therapeutic indications the compounds are administered to inhibit diseases such as cancer and inflammation but may be used to augment host biological responses in certain disease conditions. The compounds can be delivered either by the same route of administration or in combination with the anti-denatured collagen MAbs, or via different routes of administration depending on the optimum formulation, concentration and bioactive species for a patient with a specific disease. One or more of the compound species may also be used in combination with the anti-denatured collagen MAbs and formulated either together or separately and delivered by the same or different route of administration depending on the optimum formulation and bioactive species for a patient with a specific disease. The integrin derivatives may also be covalently linked to the anti-denatured collagen MAbs to achieve a beneficial therapeutic effect. Certain combinations of anti-denatured collagen antibodies and integrin species, either separate or cross-linked, can also be used in diagnostic assays.

TABLE II
Integrin Peptides, Antibodies and Derivatives

| | Antibodies, Peptide or | |
|---|---|---|
| Protein | Mimetics | Reference |
| α1β1 | Anti-VLA1 | J Clin Invest. 2002 |
| | | Dec; 110(12): 1773-82. |
| α2β1 | antibody | Br J Cancer. 1998 |
| | | Jun; 77(12): 2274-80. |
| α3β1 | antibody | Exp Cell Res. 1995 |
| | | Jul; 219(1): 233-42 |
| α4β1 | Anti-VLA-4 | Steinman L. Nat Rev Drug |
| | Natalizumab | Discov. 2005 Jun; 4(6): 510-8 |
| | Tysabri | |
| α5β1 | Volocixmab | J Biol Chem. 1997 Jul |
| | Anti-alpha5 | 11; 272(28): 17283-92 |
| α6β1 | Anti-alpha6 | Gastroenterology. 1995 |
| | | Feb; 108(2): 523-32. J Hepatol. |
| | | 1999 Oct; 31(4): 734-40 |
| α7β1 | antibodies | J Biol Chem. 1996 Oct |
| | | 11; 271(41): 25598-603 |
| α8β1 | siRNA | Cardiovasc Res. 2005 Mar |
| | | 1; 65(4): 813-22 |
| α9β1 | antibody | Mol Biol Cell. 2005 |
| | | Feb; 16(2): 861-70. Epub 2004 |
| | | Dec 1 |
| α10β1 | potential all | J Cell Sci. 2005 Mar 1; 118(Pt |
| | | 5): 929-36. Epub 2005 Feb 15. |
| α11β1 | antibody, potential all | Dev Biol. 2004 Jun |
| | | 15; 270(2): 427-42. |
| αVβ1 | Anti-CD51 (αV) | J Med Chem. 2005 Feb |
| | CNTO 95 | 24; 48(4): 1098-106 |
| | (Centocor) | |
| | Cyclic RGD peptide | |
| αVβ3 | LM609, Vitaxin | Ann Rheum Dis. 2002 Nov; 61 |
| | (Medimmune), | Suppl 2: ii96-9. |
| | (18)F-Galacto-RGD | J Nucl Med. 2005 |
| | | Aug; 46(8): 1333-41 |
| αVβ5 | antibody | Eur J Cancer. 2005 |
| | | May; 41(7): 1065-72. |
| αVβ8 | Anti-beta8 | Am J Pathol. 2003 |
| | | Aug; 163(2): 533-42 |
| αVβ6 | antibodies | Exp Cell Res. 2000 Feb |
| | | 25; 255(1): 10-7. |
| αVβ8 | RGD peptides | Exp Cell Res. 2003 Dec |
| | | 10; 291(2): 514-24. |
| α6β4 | antibodies | J Biol Chem. 2005 Mar |
| | | 4; 280(9): 8004-15. Epub 2004 |
| | | Dec 3. |
| Substituted | Chemical compounds | US patent: 6,855,722 |
| Indole | | |
| Integrin | | |
| Antagonists | | |
| Vitronectin | Chemical compounds | US patent: 6,818,201 |
| Receptor | | |
| Antagonists | | |

Therapeutic Combinations Comprising Denatured Collagen Antagonists and Cell Surface Peptides

The present application describes combinations of anti-denatured collagen antibodies, or binding peptides (shown in Table A) with peptides, antibodies, siRNA, DNA or RNA sequences that encode cell surface peptide sequences including those shown in Table III. For most therapeutic indications the compounds are administered to inhibit diseases such as cancer and inflammation but may be used to augment host biological responses in certain disease conditions. The compounds can be delivered either by the same route of administration in combination with the anti-denatured collagen MAbs or via different routes of administration depending on the optimum formulation, concentration and bioactive species for a patient with a specific disease. One or more of the compound species may also be used in combination with the anti-denatured collagen MAbs and formulated either together or separately and delivered by the same or by a different route of administration depending on the optimum formulation and bioactive species for a patient with a specific disease. The laminin derivatives may also be covalently linked to the anti-denatured collagen MAbs to achieve a beneficial therapeutic effect. Certain combinations of anti-denatured collagen antibodies and laminin species, either separate or cross-linked, can also be used in diagnostic assays.

TABLE III
Cell Surface Peptides, Antibodies and Derivatives

| | Antibodies, Peptide | |
|---|---|---|
| Protein | or Mimetics | Reference |
| erbB-2 | Herceptin, | Anticancer Res. 2005 Jul-Aug; |
| or neu | trastuzumab; peptide | 25(4): 3061-6 Cancer Res. |
| | | 2005 Aug 1; 65(15): 6891-900 |
| | | Oncology. 2001; 61 Suppl 2: 14-21 |
| CD44 | antibody | Anticancer Res. 2005 Mar-Apr; |
| | | 25(2A): 1115-21. |
| LFA-1 | Efalizumab, Raptiva | Ann Pharmacother. 2005 |
| | | Sep; 39(9): 1476-82. Epub 2005 Jul 5 |
| CD11a | Efalizumab | Am J Clin Dermatol. |
| | | 2005; 6(2): 113-8; |
| CD11b | antibody | J Infect Dis. 2005 May |
| | | 15; 191(10): 1755-60. Epub 2005 |
| | | Apr 6. |
| CD11c | antibody | Arch Dermatol Res. 1997 |
| | | Nov; 289(12): 692-7. |
| (GP) | ReoPro; abciximab | Chest. 2005 Feb; 127(2 Suppl): 53S-59S |
| IIb/IIIa | | |
| receptor | | |

Therapeutic Combinations Comprising Denatured Collagen Antagonists and Laminin

The present application describes combinations of anti-denatured collagen antibodies or binding peptides (shown in Table A) with peptides, antibodies, siRNA, DNA or RNA sequences that encode laminin peptide sequences including those shown in Table IV. For most therapeutic indications the compounds are administered to inhibit diseases such as cancer and inflammation but may be used to augment host biological responses. The compounds can be delivered either by the same route of administration in combination with the anti-denatured collagen MAbs or via different routes of administration depending on the optimum formulation, concentration and bioactive species for a patient with a specific disease. One or more of the compound species may also be used in combination with the anti-denatured collagen MAbs and formulated either together or separately and delivered by the same or a different route of administration depending on the optimum formulation and bioactive species for a patient with a specific disease. The laminin derivatives may also be covalently linked to the anti-denatured collagen MAbs to achieve is a beneficial therapeutic effect. Certain combinations of anti-denatured collagen antibodies and laminin species, either separate or cross-linked, can also be used in diagnostic assays.

TABLE IV
Laminin Peptides, Antibodies and Derivatives

| | Antibodies, Peptide | |
|---|---|---|
| Protein | or Mimetics | Reference |
| Laminin 5 | Potential for all | Curr Opin Cell Biol. 2000 |
| | | Oct; 12(5): 554-62. |
| Laminin 1 | Peptide, antibody | Endocr Relat Cancer. 2005 |
| | | Jun; 12(2): 393-406. Clin Dev |
| | | Immunol. 2005 Mar; 12(1): 67-73. |
| Laminin 8 | antibody | Breast Cancer Res. |
| | | 2005; 7(4): R411-21. Epub 2005 |
| | | Apr |
| Laminin 9 | antibody | Breast Cancer Res. |
| | | 2005; 7(4): R411-21. Epub 2005 |
| | | Apr |
| Laminin 3 | antibody | Exp Cell Res. 2000 Sep |
| | | 15; 259(2): 326-35. |
| Laminin 10 | antibody | Neoplasia. 2005 Apr; 7(4): 380-9. |
| Laminin 8-11 | antibody | J Cell Biol. 1997 May |
| | | 5; 137(3): 685-701 |

Therapeutic Combinations Comprising Denatured Collagen Antagonists and Other ECM or Secreted Peptides

The present application describes combinations of anti-denatured collagen antibodies or binding peptides (shown in Table A) with peptides, antibodies, siRNA, DNA or RNA sequences that encode other ECM proteins including those shown in Table V. For most therapeutic indications the compounds are administered to inhibit diseases such as cancer and inflammation but may be used to augment host biological responses in certain disease conditions. The compounds can be delivered either the same route of administration in combination with the anti-denatured collagen MAbs or by different routes of administration depending on the optimum formulation, concentration and bioactive species for a patient with a specific disease. One or more of the compound species may also be used in combination with the anti-denatured collagen MAbs and formulated either together or separately and delivered by the same or a different route of administration depending on the optimum formulation and bioactive species for a patient with a specific disease. The ECM derivatives may also be covalently linked to the anti-denatured collagen MAbs to achieve a beneficial therapeutic effect of having both compounds linked together. It may also be possible that certain combinations of anti-denatured collagen antibodies and ECM species, either separate or cross-linked, be used in diagnostic assays.

TABLE V
Other ECM or Secreted Peptides, Antibodies and Derivatives

| | Antibodies, Peptide | |
|---|---|---|
| Protein | or Mimetics | Reference |
| Nidogen | Antibody, potential | Eur J Biochem. 1987 Jul |
| | for all | 1; 166(1): 11-9. |
| Perlecan | Antibody, potential | Matrix. 1992 |
| | for all | Jun; 12(3): 221-32 |
| Osteopontins | Antibody, potential | Gut. 2005 |
| | for all | Sep; 54(9): 1254-62. |
| BMPs | Antibody, potential | Growth Factors. 2004 |
| | for all | Dec; 22(4): 233-41. |
| Aggrecan and | Potential for all, | Mol Cell Proteomics. |
| aggrecanase | enzyme inhibitors | 2005 Jun 21 |
| Versican | Potential for all | Cell Res. 2005 |
| | | Jul; 15(7): 483-94 |
| Decorin | Potential for all | Exp Mol Pathol. 2005 |
| | | Aug; 79(1): 68-73. |
| Biglycan | Potential for all | J Dermatol Sci. 2005 Jun |
| | | 29 |
| TSP-1 | Peptide mimetics | J Med Chem. 2005 Apr |
| | | 21; 48(8): 2838-46. |
| Fibronectin | Antibodies, peptides | Expert Opin Ther |
| | | Targets. 2005 |
| | | Jun; 9(3): 491-500. |
| VEGF | Avastin, Macugen, | Expert Opin Biol Ther. |
| | | 2005 Jul; 5(7): 997-1005 |
| Vitronectin | Antibodies, potential | J Clin Invest. 1990 |
| | for all | May; 85(5): 1372-8 |

Therapeutic Combinations Comprising Denatured Collagen Antagonists and Enzyme Inhibitors

The present application describes combinations of anti-denatured collagen antibodies/peptides (shown in Table A) with peptides, antibodies, siRNA, DNA or RNA sequences that encode other proteolytic enzymes shown in Table 6. The proteases described in Table 6 cover general classes of enzymes. For most therapeutic indications the compounds are administered to inhibit diseases such as cancer and inflammation but may be used to augment host biological responses in certain disease conditions. The compounds can be delivered either the same route of administration in combination with the anti-denatured collagen MAbs or by different routes of administration depending on the optimum formulation, concentration and bioactive species for a patient with a specific disease. One or more of the compound species may also be used in combination with the anti-denatured collagen MAbs and formulated either together or separately and delivered by the same or a different route of administration depending on the optimum formulation and bioactive species for a patient with a specific disease. The protease inhibitor derivatives may also be covalently linked to the anti-denatured collagen MAbs to achieve a beneficial therapeutic effect. Certain combinations of anti-denatured collagen antibodies and protease inhibitor species, either separate or cross-linked, can also be used in diagnostic assays.

TABLE VI
Enzyme Peptides, Antibodies and Derivatives

| | Antibodies, Peptide | |
|---|---|---|
| Protein | or Mimetics | Reference |
| MMPs | Enzyme inhibitors | Curr Cancer Drug |
| | | Targets. 2005 |
| | | May; 5(3): 203-20 |
| ADAMTS | Enzyme Inhibitors | Biochem J. 2005 Feb |
| | | 15; 386(Pt 1): 15-27 |
| Cathepsins (B, D and | Enzyme inhibitors | Neoplasma. |
| L) | | 2005; 52(3): 185-92. |
| TIMPs | peptide | Trends Mol Med. 2005 |
| | | Mar; 11(3): 97-103 Crit |
| | | Rev Oncol Hematol. 2004 |
| | | Mar; 49(3): 187-98 |
| Caspase | Protein, agonists | Oncology (Williston |
| | | Park). 2004 Nov; 18(13 |
| | | Suppl 10): 11-20 |
| uPA | Enzyme inhibitor | Neoplasma. |
| | | 2005; 52(3): 185-92. |

Therapeutic Combinations Comprising Denatured Collagen Antagonists and Immune Stimulants

The present application describes combinations of anti-denatured collagen antibodies/peptides (shown in Table A) with peptides, antibodies, siRNA, DNA or RNA sequences that encode other immune stimulants shown in Table VII. Some of the ECM proteins are referenced with names for potential therapeutic and/or diagnostic use, such as Canvaxin and Provenge. For most therapeutic indications the compounds are administered to inhibit diseases such as cancer and inflammation but may be used to augment host biological responses in certain disease conditions. The compounds can be delivered either by the same route of administration in combination with the anti-denatured collagen MAbs or via different routes of administration depending on the optimum formulation, concentration and bioactive species for a patient with a specific disease. One or more of the compound species may also be used in combination with the anti-denatured collagen MAbs and formulated either together or separately and delivered by the same or a different route of administration depending on the optimum formulation and bioactive species for a patient with a specific disease. The immune stimulant derivatives may also be covalently linked to the anti-denatured collagen MAbs to achieve a beneficial therapeutic effect. Certain combinations of anti-denatured collagen antibodies and immune stimulants, either separate or cross-linked, can also be used in diagnostic assays.

TABLE VII
Immune Stimulants

| | Antibodies, Peptide or | |
|---|---|---|
| Protein | Mimetics | Reference |
| Canvaxin | Cell-based active | Dev Biol (Basel). |
| | immunotherapy | 2004; 116: 209-17; |
| | | discussion 229-36 |
| Provenge | Peptide loaded APC | Prostate. 2004 Aug |
| | | 1; 60(3): 197-204. |
| Tumor Vaccines | Several approaches | J Clin Oncol. 2001 Mar |
| | | 15; 19(6): 1848-54 |

Therapeutic Combinations Comprising Denatured Collagen Antagonists and Tyrosine Kinase Receptor Inhibitors

The present application describes combinations of anti-denatured collagen antibodies/peptides (shown in Table A) with protease inhibitors, peptides, antibodies, siRNA, DNA or RNA sequences that encode other proteins shown in Table VIII. Some of the RTK proteins are referenced with names for potential therapeutic and/or diagnostic use, such as Tarceva, Gleevec and Iressa. For most therapeutic indications the compounds are administered to inhibit diseases such as cancer but may be used to augment host biological responses in certain disease conditions. The compounds can be delivered either the same route of administration in combination with the anti-denatured collagen MAbs or different routes of administration depending on the optimum formulation, concentration and bioactive species for a patient with a specific disease. One or more of the compound species may also be used in combination with the anti-denatured collagen MAbs and formulated either together or separately and delivered by the same or a different route of administration depending on the optimum formulation and bioactive species for a patient with a specific disease. The RTK derivatives may also be covalently linked to the anti-denatured collagen MAbs to achieve a beneficial effect. Certain combinations of anti-denatured collagen antibodies and RTK inhibitor species, either separate or cross-linked, can also be used in diagnostic assays.

TABLE VIII
Tyrosine Kinase Receptor (RTK) Inhibitors

| | Antibodies, Peptide | |
|---|---|---|
| Protein | or Mimetics | Reference |
| EGFR | Tarceva, Gefitinib | Cancer Invest. |
| | (Iressa) | 2005; 23(4): 296-302 J |
| | | Pharmacol Exp Ther. |
| | | 2005 Jul 7 |
| EGFR | Gleevec | J Pharmacol Exp Ther. |
| | | 2005 Jul 7 |
| RTK General | Enzyme inhibitor | J Pharmacol Exp Ther. |
| | | 2005 Jul 7 |

Combination of Anti-Dn Laminin Antibodies and Antibody Binding Epitopes with Other Agents for Therapeutic or Diagnostic Use.

The present application provides a combination of antagonists of denatured or proteolyzed collagens, such as but not restricted to antibodies, LMD1, LMD2, LMD5, LMD9, LMD11, LMD13, LMD14, LMD15, LMD16, LMD17, LMD21, LMD23, LMD24, LMD26, LMD30, LMD52, LMD105, LMD209, LMD418 and peptide STQ with other agents that potentially enhance the prophylactic or therapeutic profile of a single agent. The combination therapy may provide a synergistic and/or beneficial effect or may allow lower doses of a combination to provide a greater margin of safety. Encompassed herein are treatment protocols that enhance the prophylactic or therapeutic effect of an antagonist of denatured or proteolyzed laminins using a specific antibody but may be a fragment derived from an antibody, compound cross-linked or genetically fused to an antibody, peptide or mimetic epitope which binds the antibody, an antibody gene(s) using gene therapies for preventing, managing, treating or ablation of cancer or other diseases.

TABLE B
Denatured Laminin Antagonists

LMD1
LMD2
LMD5
LMD9
LMD11
LMD13
LMD14
LMD15
LMD16
LMD17
LMD21
LMD23
LMD24
LMD26
LMD30
LMD52
LMD105
LMD209
LMD418
STQ Peptide

Therapeutic Combinations Comprising Anti-Denatured Laminin Antagonists and Collagen Derived Peptides

The present application describes combinations of anti-denatured laminin antibodies or binding peptides (shown in Table B) with peptides derived from collagen peptide sequences including those shown in Table IX. Some of the collagen peptides are referenced with names for potential therapeutic and/or diagnostic use, such as Arrestin, Canstatin, etc. For most therapeutic indications the compounds are administered to inhibit diseases such as cancer and inflammation but may be used to augment host biological responses in certain disease conditions. The collagen peptides can be delivered either by the same route of administration in combination with the anti-denatured laminin MAbs or a different routes of administration depending on the optimum formulation, concentration and bioactive species for a patient with a specific disease. One or more of the peptides may also be used in combination with the anti-denatured laminin MAbs and formulated either together or separately and delivered by the same or different route of administration depending on the optimum formulation and bioactive species for a patient with a specific disease. The collagen peptides may also be covalently linked to the anti-denatured laminin MAbs to achieve a beneficial effect. Certain combinations of anti-denatured laminin antibodies and collagen peptides, either separate or cross-linked, can also be used in diagnostic assays.

TABLE IX
Collagen-Derived Peptides

| Collagen Peptide | Parent Protein | Reference |
|---|---|---|
| Human Endostatin | NC1 domanin, α1 chain of type | O′Reilly, MS et al. Cell 88, |
| | XVIII collagen | 277-285 (1997). |
| Human Endostatin- | NC10 domanin, α1 chain of type | Ramchandran, R et al, |
| like protein | XV collagen | Biochem. Biophys. Res. |
| | | Comm 255, 735-739 (1999) |
| Arrestin | NC1 domain, α1 chain of type | Kalluri et al. |
| | IV collagen | Cold Spring Harb Symp |
| | | Quant Biol. 2002; 67: 255-66 |
| Canstatin | NC1 domain, α2 chain of type | Kalluri et al. |
| Angiocol | IV collagen | Cold Spring Harb Symp |
| | | Quant Biol. 2002; 67: 255-66; |
| | | also |
| | | Biostratum Patent |
| Tumstatin | NC1 domain, α3 chain of type | Kalluri et al. |
| | IV collagen | Cold Spring Harb Symp |
| | | Quant Biol. 2002; 67: 255-66 |
| α6(IV) NC1 domain | NC1 domain, α6 chain of type | Kalluri et al. |
| | IV collagen | Cold Spring Harb Symp |
| | | Quant Biol. 2002; 67: 255-66 |

Therapeutic Combinations Comprising Anti-Denatured Laminin Antagonists and Integrin Peptides, Antibodies and Derivatives

The present application describes combinations of anti-denatured laminin antibodies/peptides (shown in Table B), with peptides, antibodies, siRNA, DNA or RNA sequences that encode integrin peptide sequences shown in Table X. Some the integrin peptides are referenced with names for potential therapeutic and/or diagnostic use, such as Tysabri, Vitaxin and ReoPro. For most therapeutic indications the compounds are administered to inhibit diseases such as cancer and inflammation but may be used to augment host biological responses in certain disease conditions. The compounds can be delivered either by the same route of administration in combination with the anti-denatured laminin MAbs or by different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The integrin derivatives may also be covalently linked to the anti-denatured collagen MAbs to achieve a potential beneficial effect. Certain combinations of anti-denatured laminin antibodies and integrin species, either separate or cross-linked, can also be used in diagnostic assays.

TABLE X
Integrin Peptides, Antibodies and Derivatives

| | Antibodies, Peptide or | |
|---|---|---|
| Protein | Mimetics | Reference |
| α1β1 | Anti-VLA1 | J Clin Invest. 2002 |
| | | Dec; 110(12): 1773-82. |
| α2β1 | antibody | Br J Cancer. 1998 |
| | | Jun; 77(12): 2274-80. |
| α3β1 | antibody | Exp Cell Res. 1995 |
| | | Jul; 219(1): 233-42 |
| α4β1 | Anti-VLA-4 | Steinman L. Nat Rev Drug |
| | Natalizumab | Discov. 2005 Jun; 4(6): 510-8 |
| | Tysabri | |
| α5β1 | Volocixmab | J Biol Chem. 1997 Jul |
| | Anti-alpha5 | 11; 272(28): 17283-92 |
| α6β1 | Anti-alpha6 | Gastroenterology. 1995 |
| | | Feb; 108(2): 523-32. J Hepatol. |
| | | 1999 Oct; 31(4): 734-40 |
| α7β1 | antibodies | J Biol Chem. 1996 Oct |
| | | 11; 271(41): 25598-603 |
| α8β1 | siRNA | Cardiovasc Res. 2005 Mar |
| | | 1; 65(4): 813-22 |
| α9β1 | antibody | Mol Biol Cell. 2005 |
| | | Feb; 16(2): 861-70. Epub 2004 |
| | | Dec 1 |
| α10β1 | potential all | J Cell Sci. 2005 Mar 1; 118(Pt |
| | | 5): 929-36. Epub 2005 Feb 15. |
| α11β1 | Antibody, potential all | Dev Biol. 2004 Jun |
| | | 15; 270(2): 427-42. |
| αVβ1 | Anti-CD51 (αV) | J Med Chem. 2005 Feb |
| | CNTO 95 (Centocor) | 24; 48(4): 1098-106 |
| | Cyclic RGD peptide | |
| αVβ3 | LM609, Vitaxin | Ann Rheum Dis. 2002 Nov; 61 |
| | (Medimmune), | Suppl 2: ii96-9. |
| | (18)F-Galacto-RGD | J Nucl Med. 2005 |
| | | Aug; 46(8): 1333-41 |
| αVβ5 | antibody | Eur J Cancer. 2005 |
| | | May; 41(7): 1065-72. |
| αVβ8 | Anti-beta8 | Am J Pathol. 2003 |
| | | Aug; 163(2): 533-42 |
| αVβ6 | antibodies | Exp Cell Res. 2000 Feb |
| | | 25; 255(1): 10-7. |
| αVβ8 | RGD peptides | Exp Cell Res. 2003 Dec |
| | | 10; 291(2): 514-24. |
| α6β4 | antibodies | J Biol Chem. 2005 Mar |
| | | 4; 280(9): 8004-15. Epub 2004 |
| | | Dec 3. |
| Substituted | Chemical compounds | U.S. Pat. No.: 6,855,722 |
| Indole Integrin | | |
| Antagonists | | |
| Vitronectin | Chemical compounds | U.S. Pat. No.: 6,818,201 |
| Receptor | | |
| Antagonists | | |

Therapeutic Combinations Comprising Anti-Denatured Laminin Antagonists and Cell Surface Peptides, Antibodies and Derivatives

The present application describes combinations of anti-denatured laminin antibodies (shown in Table B) with peptides, antibodies, siRNA, DNA or RNA sequences that encode laminin peptide sequences shown in Table XI. For most therapeutic indications the compounds are administered to inhibit diseases such as cancer and inflammation but may be used to augment host biological responses in certain disease conditions. The compounds can be delivered either the same route of administration in combination with the anti-denatured laminin MAbs or different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The laminin derivatives may also be covalently linked to the anti-denatured laminin MAbs if there is a beneficial effect of having both compounds linked together for a therapeutic effect. It may also be possible that certain combinations of anti-denatured laminin antibodies and laminin species, either separate or cross-linked, can also be used in diagnostic assays.

TABLE XI
Cell Surface Peptides, Antibodies and Derivatives

| | Antibodies, Peptide | |
|---|---|---|
| Protein | or Mimetics | Reference |
| erbB-2 or | Herceptin, | Anticancer Res. 2005 Jul-Aug; |
| neu | trastuzumab; peptide | 25(4): 3061-6 Cancer Res. |
| | | 2005 Aug 1; 65(15): 6891-900 |
| | | Oncology. 2001; 61 Suppl 2: 14-21 |
| CD44 | antibody | Anticancer Res. 2005 Mar-Apr; |
| | | 25(2A): 1115-21. |
| LFA-1 | Efalizumab, Raptiva | Ann Pharmacother. 2005 |
| | | Sep; 39(9): 1476-82. Epub 2005 |
| | | Jul 5 |
| CD11a | Efalizumab | Am J Clin Dermatol. |
| | | 2005; 6(2): 113-8; |
| CD11b | antibody | J Infect Dis. 2005 May |
| | | 15; 191(10): 1755-60. Epub 2005 |
| | | Apr 6. |
| CD11c | antibody | Arch Dermatol Res. 1997 |
| | | Nov; 289(12): 692-7. |
| (GP) IIb/IIIa | ReoPro; abciximab | Chest. 2005 Feb; 127(2 Suppl): |
| receptor | | 53S-59S |

Therapeutic Combinations Comprising Anti-Denatured Laminin Antagonists and Laminin Peptides, Antibodies and Derivatives

The present application describes combinations of anti-denatured laminin antibodies (shown in Table B) with peptides, antibodies, siRNA, DNA or RNA sequences that encode laminin peptide sequences including those shown in Table XII. For most therapeutic indications the compounds are administered to inhibit diseases such as cancer and inflammation but may be used to augment host biological responses in certain disease conditions. The compounds can be delivered either by the same route of administration in combination with the anti-laminin collagen MAbs or by different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The laminin derivatives may also be covalently linked to the anti-denatured laminin MAbs to achieve a beneficial therapeutic effect. Certain combinations of anti-denatured laminin antibodies and laminin species, either separate or cross-linked, can also be used in diagnostic assays.

TABLE XII
Laminin Peptides, Antibodies and Derivatives

| | Antibodies, Peptide | |
|---|---|---|
| Protein | or Mimetics | Reference |
| Laminin 5 | Potential for all | Curr Opin Cell Biol. 2000 |
| | | Oct; 12(5): 554-62. |
| Laminin 1 | Peptide, antibody | Endocr Relat Cancer. 2005 |
| | | Jun; 12(2): 393-406. Clin Dev |
| | | Immunol. 2005 Mar; 12(1): 67-73. |
| Laminin 8 | antibody | Breast Cancer Res. |
| | | 2005; 7(4): R411-21. Epub 2005 |
| | | Apr |
| Laminin 9 | antibody | Breast Cancer Res. |
| | | 2005; 7(4): R411-21. Epub 2005 |
| | | Apr |
| Laminin 3 | antibody | Exp Cell Res. 2000 Sep |
| | | 15; 259(2): 326-35. |
| Laminin 10 | antibody | Neoplasia. 2005 Apr; 7(4): 380-9. |
| Laminin 8-11 | antibody | J Cell Biol. 1997 May |
| | | 5; 137(3): 685-701 |

Therapeutic Combinations Comprising Anti-Denatured Laminin Antagonists and Other ECM or Secreted Peptides, Antibodies and Derivatives

The present application describes combinations of anti-denatured laminin antibodies (shown in Table B) with peptides, antibodies, siRNA, DNA or RNA sequences that encode other ECM proteins shown in Table XIII. For most therapeutic indications the compounds are administered to inhibit diseases such as cancer and inflammation but may be used to augment host biological responses in certain disease conditions. The compounds can be delivered by either the same route of administration in combination with the anti-denatured laminin MAbs or by different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The ECM derivatives may also be covalently linked to the anti-denatured laminin MAbs if there is a beneficial effect of having both compounds linked together for a therapeutic effect. It may also be possible that certain combinations of anti-denatured laminin antibodies and ECM species, either separate or cross-linked, be used in diagnostic assays.

TABLE XIII
Other ECM or Secreted Peptides, Antibodies and Derivatives

| | Antibodies, Peptide | |
|---|---|---|
| Protein | or Mimetics | Reference |
| Nidogen | Antibody, potential | Eur J Biochem. 1987 Jul |
| | for all | 1; 166(1): 11-9. |
| Perlecan | Antibody, potential | Matrix. 1992 |
| | for all | Jun; 12(3): 221-32 |
| Osteopontins | Antibody, potential | Gut. 2005 |
| | for all | Sep; 54(9): 1254-62. |
| BMPs | Antibody, potential | Growth Factors. 2004 |
| | for all | Dec; 22(4): 233-41. |
| Aggrecan and | Potential for all, | Mol Cell Proteomics. |
| aggrecanase | enzyme inhibitors | 2005 Jun 21 |
| Versican | Potential for all | Cell Res. 2005 |
| | | Jul; 15(7): 483-94 |
| Decorin | Potential for all | Exp Mol Pathol. 2005 |
| | | Aug; 79(1): 68-73. |
| Biglycan | Potential for all | J Dermatol Sci. 2005 Jun |
| | | 29 |
| TSP-1 | Peptide mimetics | J Med Chem. 2005 Apr |
| | | 21; 48(8): 2838-46. |
| Fibronectin | Antibodies, peptides | Expert Opin Ther |
| | | Targets. 2005 |
| | | Jun; 9(3): 491-500. |
| VEGF | Avastin, Macugen, | Expert Opin Biol Ther. |
| | | 2005 Jul; 5(7): 997-1005 |
| Vitronectin | Antibodies, potential | J Clin Invest. 1990 |
| | for all | May; 85(5): 1372-8 |

Therapeutic Combinations Comprising Anti-Denatured Laminin Antagonists and Enzyme Inhibitors

The present application describes combinations of anti-denatured laminin antibodies/peptides (shown in Table B) with peptides, antibodies, siRNA, DNA or RNA sequences that encode other proteolytic enzymes shown in Table XIV. The proteases described in Table XIV cover general classes of enzymes of which there are several members of each class. For most therapeutic indications the compounds are administered to inhibit diseases such as cancer and inflammation but may be used to augment host biological responses in certain disease conditions. The compounds can be delivered either by the same route of administration in combination with the anti-denatured laminin MAbs or by different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The protease inhibitor derivatives may also be covalently linked to the anti-denatured laminin MAbs if there is a beneficial effect of having both compounds linked together for a therapeutic effect. It may also be possible that certain combinations of anti-denatured laminin antibodies and protease inhibitor species, either separate or cross-linked, be used in diagnostic assays.

TABLE XIV
Enzyme Peptides, Antibodies and Derivatives

| | Antibodies, Peptide | |
|---|---|---|
| Protein | or Mimetics | Reference |
| MMPs | Enzyme inhibitors | Curr Cancer Drug |
| | | Targets. 2005 |
| | | May; 5(3): 203-20 |
| ADAMTS | Enzyme Inhibitors | Biochem J. 2005 Feb |
| | | 15; 386(Pt 1): 15-27 |
| Cathepsins (B, D and | Enzyme inhibitors | Neoplasma. |
| L) | | 2005; 52(3): 185-92. |
| TIMPs | peptide | Trends Mol Med. 2005 |
| | | Mar; 11(3): 97-103 Crit |
| | | Rev Oncol Hematol. 2004 |
| | | Mar; 49(3): 187-98 |
| Caspase | Protein, agonists | Oncology (Williston |
| | | Park). 2004 Nov; 18(13 |
| | | Suppl 10): 11-20 |
| Upa | Enzyme inhibitor | Neoplasma. |
| | | 2005; 52(3): 185-92. |

Therapeutic Combinations Comprising Anti-Denatured Laminin Antagonists and Immune Stimulants

The present application describes combinations of anti-denatured laminin antibodies/peptides (shown in Table B) with peptides, antibodies, siRNA, DNA or RNA sequences that encode other immune stimulants shown in Table XV. Some of the ECM proteins are referenced with names for potential therapeutic and/or diagnostic use, such as Canvaxin and Provenge. For most therapeutic indications the compounds are administered to inhibit diseases such as cancer and inflammation but may be used to augment host biological responses in certain disease conditions. The compounds can be delivered either by the same route of administration in combination with the anti-denatured laminin MAbs or by different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The immune stimulant derivatives may also be covalently linked to the anti-denatured laminin MAbs if there is a beneficial effect of having both compounds linked together for a therapeutic effect. It may also be possible that certain combinations of anti-denatured laminin antibodies and immune stimulants, either separate or cross-linked, be used in diagnostic assays.

TABLE XV
Immune Stimulants

| | Antibodies, Peptide or | |
|---|---|---|
| Protein | Mimetics | Reference |
| Canvaxin | Cell-based active | Dev Biol (Basel). |
| | immunotherapy | 2004; 116: 209-17; |
| | | discussion 229-36 |
| Provenge | Peptide loaded APC | Prostate. 2004 Aug |
| | | 1; 60(3): 197-204. |
| Tumor Vaccines | Several approaches | J Clin Oncol. 2001 Mar |
| | | 15; 19(6): 1848-54 |

Therapeutic Combinations Comprising Anti-Denatured Laminin Antagonists and Tyrosine Kinase Receptor Inhibitors

The present application describes combinations of anti-denatured laminin antibodies/peptides (shown in Table B) with protease inhibitors, peptides, antibodies, siRNA, DNA or RNA sequences that encode other tyrosine kinase (RTK) proteins shown in Table XVI. Some of the RTK proteins are referenced with names for potential therapeutic and/or diagnostic use, such as Tarceva, Gleevec and Iressa. For most therapeutic indications the compounds are administered to inhibit diseases such as cancer but may be used to augment host biological responses in certain disease conditions. The compounds can be delivered either the same route of administration in combination with the anti-denatured laminin MAbs or different routes of administration depending on the optimum formulation, concentration and bioactive species for a patient with a specific disease. One or more of the compound species may also be used in combination with the anti-denatured laminin MAbs and formulated either together or separately and delivered by the same or different route of administration depending on the optimum formulation and bioactive species for a patient with a specific disease. The RTK derivatives may also be covalently linked to the anti-denatured laminin MAbs if there is a beneficial effect of having both compounds linked together for a therapeutic effect. It may also be possible that certain combinations of anti-denatured laminin antibodies and RTK inhibitor species, either separate or cross-linked, be used in diagnostic assays.

TABLE XVI
Tyrosine Kinase Receptor (RTK) Inhibitors

| | Antibodies, Peptide | |
|---|---|---|
| Protein | or Mimetics | Reference |
| EGFR | Tarceva, Gefitinib | Cancer Invest. |
| | (Iressa) | 2005; 23(4): 296-302 J |
| | | Pharmacol Exp Ther. |
| | | 2005 Jul 7 |
| EGFR | Gleevec | J Pharmacol Exp Ther. |
| | | 2005 Jul 7 |
| RTK General | Enzyme inhibitor | J Pharmacol Exp Ther. |
| | | 2005 Jul 7 |

ECM Components

As used herein, an “ECM component” is a component of the non-cellular compartment. ECM components include, e.g., fibrin, fibrinogen, vitronectin, von Willebrand's factor, osteospontin, bone sialoprotein I, collagen, laminin, elastin, thrombospondin, tenascin, osteopontin, and fibronectin, as well as other proteins and molecules found in association with these ECM components or found in the same location as these ECM components (Gustafsson, E., et al., R. Exp. Cell Res. 2000, 261:52-68; Werb, Z., et al., Ann. N.Y. Acad. Sci. 1998, 857:110-118, and; Heissig, et al., Curr. Opin. Hematol. 2003, 10:136-141).

An epitope is that amino acid sequence or sequences that are recognized by an antagonist, e.g., an antibody antagonist or fragment thereof as provided herein. An epitope can be a linear peptide sequence or can be composed of non-contiguous amino acid sequences. An antagonist can recognize one or more sequences, therefore an epitope can define more than one distinct amino acid sequence target. The epitopes recognized by an antagonist can be determined by peptide mapping and sequence analysis techniques well known to one of skill in the art.

A “cryptic epitope of an ECM component” is an epitope of an ECM component protein sequence that is not exposed for recognition within a native ECM component, but is capable of being recognized by an antagonist of a denatured or proteolyzed ECM component. Sequences that are not exposed, or are only partially exposed, in the native structure are potential cryptic epitopes. If an epitope is not exposed, or only partially exposed, then it is likely that it is buried within the interior of the molecule. The sequence of cryptic epitopes can be identified by determining the specificity of an antagonist. Candidate cryptic epitopes also can be identified, for example, by examining the three-dimensional structure of a native ECM component.

It is likely that the degradation of a number of ECM molecules results in exposure of sites that play an essential role in angiogenesis and tumor growth. Recent studies suggest that distinct domains present within ECM proteins regulate angiogenesis and tumor growth. Fragments of molecules such as plasminogen (i.e. angiostatin), collagen-XVIII (i.e. endostatin), collagen-XV (i.e. Restin), MMP-2 (i.e. PEX-domain), fibronectin, and thrombospondin have all been shown to regulate angiogenesis (O'Reilly, M. S., Holmgren, L., Shing, Y., Chen, C., Rosenthal, R. A., Moses, M., Lane, W. S., Cao, Y., Sage, E. H., and Folkman, J. (1994) Cell 79(2), 315-28; O'Reilly, M. S., Boelm, T., Shing, Y., Fukai, N., Vasios, G., Lane, W. S., Flynn, E., Birkhead, J. R., Olsen, B. R., and Folkman, J. (1997) Cell 88(2), 277-85; Ramchandran, R., Dhanabal, M., Volk, R., Waterman, M. J., Segal, M., Lu, H., Knebelmann, B., and Sukhatme, V. P. (1999) Biochemical & Biophysical Research Communications 255(3), 735-9; Brooks, P. C., Silletti, S., von Schalscha, T. L., Friedlander, M., and Cheresh, D. A. (1998) Cell 92(3), 391-400; Castellani, P., Viale, G., Dorcaratto, A., Nicolo, G., Kaczmarek, J., Guerze, G., and Zardi, L. (1994) International Journal of Cancer 59(5), 612-8; Tolsma, S. S., Volpert, O. V., Good, D. J., Frazier, W. A., Polyerini, PlJ., and Bouck, N. (1993) Journal of Cell Biology 122(2), 497-511).

Collagens

Collagens, for example, may be from any mammal such as rat, mouse, pig, rabbit etc. or from a bird such as chicken. Generally, a collagen is an extracellular matrix protein containing a [Gly-Xaa-Xaa]ₙ sequence. Collagen types are well known in the art (see, e.g., Olsen, B. R. 1995, Curr. Op. Cell. Biol. 5:720-727; Kucharz, E. J. The Collagens: Biochemistry and Pathophysiology. Springer-Verlag, Berlin, 1992; Kunn, K. in Structure and Function of Collagen Types, eds. R. Mayne and R. E. Burgeson, Academic Press, Orlando; U.S. Pub. No. 2003/0113331). Human collagens are preferred collagens. Denatured collagen refers to collagen that has been treated such that it no longer predominantly assumes the native triple helical form. Denaturation can be accomplished by heating the collagen. In one embodiment, collagen is denatured by heating for about 15 minutes at about 100° C. Denaturation also can be accomplished by treating the collagen with a chaotropic agent. Suitable chaotropic agents include, for example, guanidinium salts. Denaturation of a collagen can be monitored, for example, by spectroscopic changes in optical properties such as absorbance, circular dichroism or fluorescence of the protein, by nuclear magnetic resonance, by Raman spectroscopy, or by any other suitable technique. Denatured collagen refers to denatured full-length collagens as well as to fragments of collagen. A fragment of collagen can be any collagen sequence shorter than a native collagen sequence. For fragments of collagen with substantial native structure, denaturation can be effected as for a native full-length collagen. Fragments also can be of a size such that they do not possess significant native structure or possess regions without significant native structure of the native triple helical form. Such fragments are denatured all or in part without requiring the use of heat or of a chaotropic agent. The term denatured collagen encompasses proteolyzed collagen.

Proteolyzed collagen refers to a collagen that has been fragmented through the action of a proteolytic enzyme. In particular, proteolyzed collagen can be prepared by treating the collagen with a metalloproteinase, such as MMP-1, MMP-2 or MMP-9, or by treating the collagen with a cellular extract containing collagen degrading activity. Proteolyzed collagen can also be that which occurs naturally at sites of ECM remodeling in a tissue.

Potentially useful antagonists that specifically bind to cryptic epitopes of ECM components have been described in the literature. Antibody antagonists of cryptic collagen epitopes are described in U.S. Publication No. 2003/0113331, U.S. Publication No. 2004/0242490 A1 and WO 2004/073649, all incorporated herein by reference. Antibody antagonists, including mAbs HUI77, HUIV26 and XL313, described in U.S. Publication No. 2003/0113331 bind to a denatured collagen or collagens, and are reported to bind with substantially reduced affinity to the native form of the collagen or collagens. Antagonists useful in the methods provided herein can have an affinity to the native form of collagen, or another ECM component, of about 1.5-fold lower than that for the denatured collagen or denatured ECM component. Antagonists as described herein are, preferably, specific for any one of the denatured collagens, e.g., types-I, II, III, IV, V, VI, VII, VIII, IX, X, and combinations thereof.

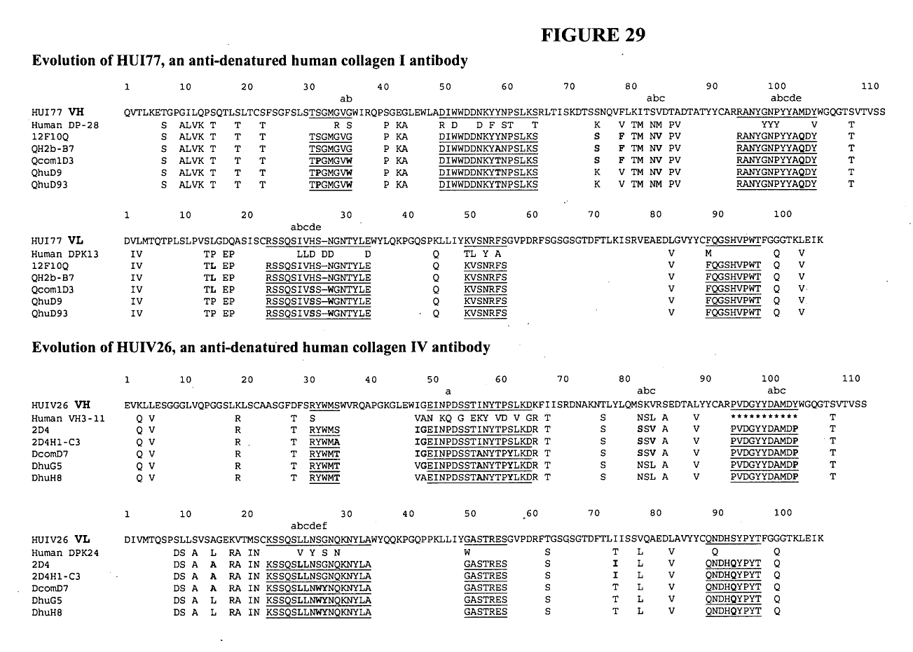

The CDRs of HUI77 and HUIV26 humanized antibodies are shown in FIG. 29 and described in the description of the figure. These antibodies, which were either partially or fully humanized according to methods known to those of skill in the art, bind to denatured collagen. Description of these antibodies, related antibodies, and their production are provided in U.S. Ser. No. 10/011,250, and US Publication No. 2004/0091482, incorporated herein by reference in their entirety.

Antibodies to denatured collagen have been identified and deposited with the ATCC (Manassas, Va.) under the following accession numbers: Hybridoma HUIV26 (PTA-6563), Hybridoma HUI177 (PTA-6551) and Hybridoma XL313 (PTA-6552).

In U.S. Publication No. 2004/0242490 A1 and WO 2004/073649, “CLK-Peptide and SLK-Peptide,” antagonists to denatured collagen, CLK-peptide and SLK-peptide, are reported as having a binding affinity to denatured collagen type-IV that is substantially greater than the binding affinity of the antagonist to native collagen type-IV. A “substantially greater affinity” is defined therein as a binding affinity at least 1.5-fold greater for the target compound (denatured collagen) as compared to the standard compound (native collagen).

A preferred denatured collagen type-IV selective peptide antagonist contemplated for use in the compositions and methods provided herein is the CLK-peptide. CLK-peptide binds to denatured collagen type-IV with high specificity. The amino acid sequence of CLK peptide is NH2-C-L-K-Q-N-G-G-N—F—S-L-G-COOH (SEQ ID NO: 29). CLK-peptide binds to regions within denatured collagen type-IV and inhibits cellular interactions with denatured collagen type-IV.

Another selective denatured collagen type-IV peptide antagonist contemplated for use in the compositions and methods provided herein is SLK-peptide. SLK-peptide binds with high specificity to denatured collagen type-IV and inhibits cellular interactions with denatured collagen type-IV. The amino acid sequence of SLK-peptide is NH2-S-L-K-Q-N-G-G-N—F—S-L-C—COOH (SEQ ID NO: 30).

A further preferred selective denatured collagen type-IV peptide antagonist contemplated for use in the compositions and methods provided herein is KGGCLK peptide (residues 1-6 of SEQ ID NO: 31). KGGCLK peptide (residues 1-6 of SEQ ID NO: 31) binds with high specificity to denatured collagen type-IV and inhibits cellular interactions with denatured collagen type-IV. The amino acid sequence of KGGCLK peptide is NH2-K-G-G-C-L-K-Q-N-G-G-N—F—S-L-G-G-K—COOH (SEQ ID NO: 31).

Laminins

Laminins are a large family of extracellular matrix glycoproteins. Laminins have been shown to promote cell adhesion, cell growth, cell migration, cell differentiation, neurite growth, and to influence the metastatic behavior of tumor cells (U.S. Pat. No. 5,092,885). Laminin, of which there are at least ten isoforms, is a major component of basement membranes and has been shown to mediate cell-matrix attachment, gene expression, tyrosine phosphorylation of cellular proteins, and branching morphogenesis (Streuli, et al., J. Cell Biol. 1993, 129:591-603; Malinda and Kleinman, Int. J. Biochem. Cell Biol. 1996, 28:957-1959; Timpl and Brown, Matrix Biol. 1994, 14:275-281; Tryggvason, Curr. Op. Cell Biol. 1993 5:877-882; Stahl, et al., J. Cell Sci. 1997, 110:55-63). Laminin binds to type IV collagen, heparin, gangliosides, and cell surface receptors and promotes the adhesion and growth of various epithelial and tumor cells as well as neurite outgrowth. Laminin is thought to mediate cell-matrix interactions and to be a structural component of all basement membranes binding to collagen IV, heparin sulfate proteoglycan, and nidogen-entactin. The laminin molecule is composed of three polypeptide chains (α, β, and γ) assembled into a cross-shaped structure. Different α, β, and γ chains may be combined, which accounts for the large size of the laminin family (Jones, J. C. R. et al., Micr. Res. Tech. 2000, 51:211-213; Patarroyo, M. et al., Semin. Cancer Biol. 2002, 12:197-207).

The extracellular protein laminin is a heterotrimeric molecule found primarily, but not exclusively, in the basement membrane (Jones, J. C. R. et al., Micr. Res. Tech. 2000; 51:211-213; Patarroyo, M. et al., Semin. Cancer Biol. 2002; 12:197-207). Each laminin molecule has one copy each of a α-laminin chain, a β-laminin chain, and a γ-laminin chain. There are at least five different alpha chains (molecular weight approximately 400 kDa), three different beta chains (molecular weight approximately 200 kDa), and three different gamma chains of laminin (molecular weight approximately 200 kDa). If they assembled randomly, there would be 45 possible isoforms, but the assembly appears to be biased to favor certain combinations. For example, as far as it is known, the γ2-chain only assembles with the α3- and β3-chains, thereby forming the combination known as laminin-5 (Tunggal et al., Microsc. Res. Tech. (2000) 51:214-27). As of 2000, twelve (12) isoforms had been characterized. The table below lists the characteristics of these isoforms together with associated GenBank accession numbers.

| | | | | alpha | beta |
|---|---|---|---|---|---|
| Laminin 1 | α1 | β1 | γ1 | P25391 | P07942 |
| Laminin 2 | α2 | β1 | γ1 | | P07942 |
| Laminin 3 | α1 | β2 | γ1 | P25391 | |
| Laminin 4 | α2 | β2 | γ1 | | |
| Laminin 5 | α3 | β3 | γ2 | | |
| Laminin 6 | α3 | β1 | γ1 | | P07942 |
| Laminin 7 | α3 | β2 | γ1 | | |
| Laminin 8 | α4 | β1 | γ1 | | P07942 |
| Laminin 9 | α4 | β2 | γ1 | | |
| Laminin 10 | α5 | β1 | γ1 | O15230 | P07942 |
| Laminin 11 | α5 | β2 | γ1 | O15230 | |
| Laminin 12 | α2 | β1 | γ3 | P24043 | P07942 |

Based on structural studies, generally the carboxy-terminal regions of the beta and gamma chains associate a portion of the alpha chain in a long rod-like structure. The amino-terminal domains project from the trimeric structure as monomeric arms and the carboxy-terminal region continues out from the trimeric structure as a globular G domain.

Antagonists of cryptic laminin epitopes are disclosed in U.S. Publication No. 2004/224896 and WO 2004/087734. In U.S. Publication No. 2004/224896 and WO 2004/087734, peptide antagonists of denatured laminin are also described as having a binding affinity to denatured laminin that is “substantially greater” than the binding affinity of the antagonists to native laminin. “Substantially greater affinity” is defined therein as a binding affinity at least 1.5-fold greater for the target compound as compared to the standard compound and, more preferably, at least 10-fold greater and, most preferably, at least 100-fold greater. The selective antagonists are specific for denatured laminin (the target compound) and the binding affinities of the selective antagonists are compared to native laminin (the standard compound).

Denaturation of laminin can be accomplished similarly to that described for collagen, and is discussed in U.S. Publication No. 2004/224896 and WO 2004/087734.

One denatured laminin antagonist described in these publications having the amino acid sequence NH2-S-T-Q-N-A-S-L-L-S-L-T-V—C—COOH (SEQ ID NO: 32). Another preferred denatured laminin selective antagonist for use in the compositions and methods provided herein is a peptide having the amino acid sequence NH2-K-G-G-C—S-T-Q-N-A-Q-L-L-S-L-I—V-G-K-A-COOH (STQ-peptide; SEQ ID NO: 33). Another preferred denatured laminin selective antagonist for use in the compositions and methods provided herein is a peptide having the amino acid sequence NH2-K-G-G-S-T-Q-N-A-Q-L-L-S-L-I—V-G-K-A-COOH (STQ-peptide-S; SEQ ID NO: 34).

Antibodies to denatured laminin, denatured collagen, fibronectin, and other substrates, can be obtained by subtractive immunization methods described herein and in the art. For example, subtractive immunization can be used to isolate monoclonal antibodies that react specifically with the denatured forms of human laminin.

Antibodies to denatured laminin have been identified and deposited with the ATCC under the following accession numbers:

Also provided herein are methods for screening antagonists that bind specifically to a denatured laminin or laminins, but bind with substantially reduced affinity to the native form of the laminin or laminins. Such antagonists may be used to inhibit angiogenesis.

In one aspect, the antagonist specifically binds to a denatured laminin or laminins but binds to the native form of each of the aforementioned laminin or laminins with substantially reduced affinity. In some embodiments, the antagonist has at least about a two-fold increased binding to denatured laminin when compared to its binding to native laminin. In another embodiment, the antagonist has at least about a five-fold increase in binding between denatured laminin compared to its binding to native laminin. In still another embodiment, the antagonist has at least about a six-fold increase in binding between denatured laminin compared to its binding to native laminin. In a further embodiment, the antagonist has at least about a ten-fold increase in binding between denatured laminin compared to its binding to native laminin.

In another aspect, the antagonist specifically binds to a backbone domain in denatured laminin but binds the aforementioned backbone domain in the native form of said laminin with an affinity that is less by a factor of at least about two. In an embodiment, the antagonist that specifically binds to a backbone domain in denatured laminin but binds the aforementioned backbone domain in the native form of said laminin with an affinity that is less by a factor of at least about five. In another embodiment, the antagonist that specifically binds to a backbone domain in denatured laminin but binds the aforementioned backbone domain in the native form of said laminin with an affinity that is less by a factor of at least about six. In a further embodiment, the antagonist that specifically binds to a backbone domain in denatured laminin but binds the aforementioned backbone domain in the native form of said laminin with an affinity that is less by a factor of at least about ten.

In a further embodiment of the invention, the antagonist is a monoclonal antibody. In non-limiting embodiments, the antagonist is a monoclonal antibody having the binding specificity of monoclonal antibody clone LMD2, LMD9, LMD21, LMD24, LMD52, LMD105, LMD1, LMD5, LMD11, LMD17, LDM26, LMD13, LMD14, LMD15, LMD16, LMD23, LMD30, LMD209, or LMD418. In other embodiments, the antagonist is a polyclonal antibody. In further embodiments, the antagonist is a humanized monoclonal antibody. In still further embodiments, the antagonist is a chemically modified monoclonal antibody. In yet other embodiments, the antagonist is a fragment of a monoclonal antibody, polyclonal antibody, humanized monoclonal antibody or a chemically modified monoclonal antibody.

In another embodiment, the antagonist is a polypeptide, a linear peptide, or a cyclic peptide. In other embodiments, the antagonist is a non-peptidic compound. In further embodiments, the antagonist is, for example, an oligonucleotide, a carbohydrate, a lipid, or a synthetic polymer.

In an additional aspect, the invention features an epitope of laminin wherein the epitope is bound by an antibody or antagonist of the invention with a greater affinity in denatured laminin than the antibody or antagonist binds native the epitope in native laminin. The epitope may be a purified or isolated peptide. The epitope may comprise amino acids other than those of the epitope. The epitope may also consist essentially of the amino acids of the epitope. The epitope may also consist of the amino acids of the epitope. If the epitope comprises amino acids in addition to those of the epitope, it may comprise about 1 to 10 additional amino acids, it may comprise about 10 to 33 additional amino acids, it may comprise about 30 to 50 additional amino acids, it may comprise about 40 to 66 additional amino acids, or it may comprise about 50 to 100 additional amino acids, or it may comprise more than about 100 additional amino acids.

An antagonist may be an antibody, or functional fragment thereof, that binds via its variable region with denatured laminin but binds to a substantially lesser extent with the native form of the laminin. The antibodies provided herein can be monoclonal or polyclonal; means for isolating such antibodies are described below. An antagonist can also be a non-antibody molecule capable of specifically binding denatured laminin, but binding a native form of the laminin with less affinity. Such non-antibody antagonists also can be other proteins, other polypeptides, or non-peptidic compounds such as a small organic molecules, carbohydrates, or oligonucleotides.

Antibodies to denatured collagen have been identified and deposited with the ATCC (Manassas, Va.) under the following accession numbers: Hybridoma LMD-9 (PTA-6586), Hybridoma LMD-11 (PTA-6587), and Hybridoma LMD-17 (PTA-658).

Matrix Metalloproteases (MMPs)

Matrix metalloproteases (MMPs) play a role in the proteolytic remodeling of the extracellular matrix (ECM). The synthesis of connective tissues is in dynamic equilibrium with the degradation of the extracellular matrix. That degradation is due, in part, to MMPs, a family of proteases (enzymes) involved in the degradation and remodeling of connective tissues. Members of this family of endopeptidase enzymes are secreted as proenzymes from various cell types that reside in or are associated with connective tissue, such as fibroblasts, monocytes, macrophages, endothelial cells, and invasive or metastatic tumor cells. MMP expression is stimulated by growth factors and cytokines in the local tissue environment, where these enzymes act to specifically degrade protein components of the extracellular matrix, such as collagen, proteoglycans (protein core), fibronectin and laminin. These ubiquitous extracellular matrix components are present in the linings of joints, interstitial connective tissues, basement membranes and cartilage. The MMPs share a number of properties, including zinc and calcium dependence, secretion as zymogens, and 40-50% amino acid sequence homology.

Excessive degradation of extracellular matrix by MMPs is implicated in the pathogenesis of many diseases of both chronic and acute nature. For example, numerous studies, as reviewed in Exp. Opin. Invest. Drugs, 5, 323-335, (1996), have established that expression and activation of MMPs are critical events in tumor growth, invasion and metastasis. In addition, MMP activity has been found to be required for angiogenesis, which is necessary for tumor growth as well as for other pathological conditions such as macular degeneration.

The members of this family of enzymes include, but are not limited to, collagenases (MMP-1), gelatinases or collagenases of type IV (MMP-2, MMP-9), matrilysin (MMP-7, PUMP-1), and stromelysins (MMP-3).

U.S. Publication No. 2002/0182215, incorporated herein by reference in its entirety, describes peptide antagonists of MMP-2 potentially useful in the methods of the present invention. In particular, this publication describes compositions for inhibiting angiogenesis comprising a peptide that contains a specific amino acid sequence of MMP-2. The peptide, defined by amino acid positions 582-590 of MMP-2, potently inhibits angiogenesis and tumor growth. It comprises the following amino acid sequence: Ile-Phe-Ala-Gly-Asp-Lys-Phe-Trp-Arg (SEQ ID NO: 35).

The publication also describes a peptide having the above sequence flanked by other amino acids. For, example, the 9 amino acid sequence may be flanked by cysteine or other residues at the amino and carboxy termini as follows: Cys-Ile-Phe-Ala-Gly-Asp-Lys-Phe-Trp-Arg-Cys (termed “Pexstatin;” SEQ ID NO: 36).

The application further provides compositions for inhibiting angiogenesis or tumor growth comprising organic and non-peptidic mimetics based on the above amino acid sequence as well as optimized sequences flanking the region of MMP-2 within which the sequence lies. It also provides compositions for inhibiting angiogenesis comprising agonists of a site defined by a specific amino acid sequence of MMP-2. Further, the invention provides antagonists for inhibiting angiogenesis directed specifically to a specific site within MMP-2 that include organic and peptidic or non-peptidic mimetics of the epitope.

Protein-protein interactions involving certain sequences within the proteolytic enzyme MMP-9 and/or β1 integrin receptors have been reported to contribute to angiogenesis and/or tumor growth by localizing the proteolytic activity to the cell surface. Therefore, modifying protein-protein interactions involving certain sequences found within the MMP-9 and/or β1 integrin receptors can inhibit angiogenesis and/or tumor growth.

The gelatinase MMP-9 is a 92-kD enzyme released by mononuclear phagocytes, neutrophils, corneal epithelial cells, tumor cells, cytotrophoblasts and keratinocytes. There is evidence that MMP-9 binds directly with the β1 integrin, α5β1 integrin, and reports that MMP-9 and α5β1 integrin are closely associated within both the human vascular compartment as well as on the tumor cells themselves suggest that MMP-9 and α5β1 integrin may co-localize on the surface of a cell and blood vessels.

Inhibitors of MMP-9-β1 integrin interactions are contemplated for use in the combination therapies of the invention. WO 01/004157, entitled “Novel Method and Composition for Inhibition of Angiogenesis Using Antagonists Based on MMP-9 and β1 Integrins,” to Brooks et al., incorporated herein in its entirety by reference, describes a polypeptide identified as FRIP-1 for mediating the interaction between MMP-9 and α5β1 integrin, and demonstration that FRIP-1 inhibits angiogenesis.

The publication describes the use of FRIP-1 to identify antagonists for modifying the protein-protein interactions involving certain amino acid sequences within MMP-9 and/or α5β1 integrin. By injecting mice with FRIP-1 conjugated to a carrier protein, a monoclonal antibody, FM155, was identified. The mAb FM155 was found to potently inhibit tumor growth in vivo. It was found to have a high specificity for FRIP-1 but did not react with the control peptide AAAA.

WO 01/004157 also describes methods for generating other antagonists including, but not limited to, peptides, polypeptides, non-peptidic molecules, for example, organic molecules and oligonucleotides, proteins, enzymes, antibodies, monoclonal and polyclonal, etc. The antagonists described bind to FRIP-1 but bind to the control peptide AAAA with substantially reduced affinity. Apparent affinities can be determined by methods such as an enzyme linked immunosorbent assay (ELISA) or any other technique familiar to one of skill in the art. True affinities can be measured by techniques known to one of skill in the art.

Other antagonists that compete at the epitope defined by Mab FM155 can have similar anti-angiogenic and anti-tumor activities. Such antagonists include additional function blocking mAbs, humanized Mabs, chimeric Mabs, toxin conjugated Mabs, polyclonal antibodies, small peptide antagonists directed to this epitope, as well as organic and non-peptidic mimetics of the epitope defined by FM155. In addition, the epitopes defined by the monoclonal antibody FM155 may themselves function as potent anti-angiogenic and/or anti-tumor compounds. Moreover, peptides containing epitopes recognized by an antagonist can be used themselves.

Antagonists can specifically modify protein-protein interactions, wherein the protein-protein interactions comprise interactions between at least one amino acid sequence within a first protein and at least one amino acid sequence within a second protein. The first protein of such an antagonist can be MMP-9 or it can be a β1-containing integrin. Alternatively, the first protein can be MMP-9 and the second protein can be a β1-containing integrin. Further, in such a case, the protein-protein interactions may be such as to cause MMP-9 to bind to the β1-containing integrin.

Alternatively, when the first protein is a β1-containing integrin it can be α5β1 integrin or when the second protein is a β1-containing integrin it can be α5β1 integrin.

The antagonist can also be a polypeptide, a linear peptide, or a cyclic peptide. It can also be a non-peptidic compound, for example, a small organic compound or an oligonucleotide. The antagonist can further be conjugated to cytotoxic or cytostatic agents.

The polypeptide for inhibiting angiogenesis or tumor growth can specifically bind to MMP-9 with a binding capacity significantly greater than the binding capacity of the control peptide to MMP-9. In another embodiment, the invention is a polypeptide for inhibiting angiogenesis or tumor growth wherein the polypeptide specifically binds to a β1 containing integrin with a binding capacity significantly greater than the binding capacity of the control peptide to the β1-containing integrin.

The peptide can also comprise a sequence encoding an epitope recognized by an antagonist that specifically modifies protein-protein interactions, wherein the protein-protein interactions comprise interactions between at least one amino acid sequence within a first protein and at least one amino acid sequence within a second protein. For example, the antagonist can be a monoclonal antibody (e.g., mAb FM155) or a peptide.

The antagonists can be antibodies, which, in the general case, modify protein-protein interactions involving certain amino acid sequences within MMP-9 and/or β1 integrin. Such antibodies could include antibodies that bind to a peptide with the polypeptide sequence of FRIP-1, but that do not bind to the control peptide sequence. The antibody antagonists also can inhibit angiogenesis. Cell lines which produce the antibodies, methods for producing the cell lines, and methods for producing the monoclonal antibodies are also included.

The antibodies can be monoclonal or polyclonal. A monoclonal antibody would comprise antibody molecules that immunoreact with MMP-9 and α5β1 integrin. Antibody antagonists of the invention can be generated according to a number of methods known to one of skill in the art. For example, an animal can be immunized with FRIP-1 or fragment thereof. Antibodies thus generated can be selected both for their ability to bind to FRIP-1 but not to bind to the control peptide. The monoclonal antibody can also have the immunoreaction characteristics of FM155.

Polypeptide antagonists of the localization of MMP-9 to the cell surface can be any peptide or polypeptide capable of modifying protein-protein interactions involving certain amino acid sequences within MMP-9 and/or β1 integrin. The identification of preferred antagonist peptides having selectivity for MMP-9 or β1 integrins can readily be identified in a typical inhibition of binding assay, such as the ELISA assay.

As described elsewhere, peptide and polypeptide antagonists can be generated by a number of techniques known to one of skill in the art. For example, a two hybrid system (e.g., Fields, S. (1989) Nature 340:245-6) can use a fragment of MMP-9 as “bait” for selecting protein antagonists from a library that bind to the FRIP-I. The library of potential antagonists can be derived from a cDNA library, for example. In another embodiment, the potential antagonists can be variants of known MMP-9 binding proteins. Such proteins can be randomly mutagenized or subjected to gene shuffling, or other available techniques for generating sequence diversity.

As also described elsewhere, peptide and polypeptide antagonists can be generated by techniques of molecular evolution. Protein pools representing numerous variants can be selected for their ability to bind to FRIP-1, for instance by passing such protein pools over a solid matrix to which a FRIP-1 has been attached. Elution with gradients of salt, for example, can provide purification of variants with affinity for the FRIP-1. A negative selection step also can be included whereby such pools are passed over a solid matrix to which the control peptide AAAA has been attached. The filtrate will contain those variants within the pool that have a reduced affinity for the AAAA.

Peptide and polypeptide antagonists of the invention also can be generated by phage display. A randomized peptide or protein can be expressed on the surface of a phagemid particle as a fusion with a phage coat protein. Techniques of monovalent phage display are widely available (see, e.g., Lowman H. B. et al. (1991) Biochemistry 30:10832-8.) Phage expressing randomized peptide or protein libraries can be panned with a solid matrix to which a AAAA molecule has been attached. Remaining phage do not bind AAAA, or bind AAAA with substantially reduced affinity. The phage are then panned against a solid matrix to which a FRIP-1 has been attached. Bound phages are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In another embodiment, a polypeptide includes any analog, fragment or chemical derivative of a polypeptide whose amino acid residue sequence is shown herein so long as the polypeptide is an antagonist of FRIP-1 but not of the control peptide. Therefore, a present polypeptide can be subject to various changes, substitutions, insertions, and deletions where such changes provide for certain advantages in its use. In this regard, a FRIP-1 antagonist polypeptide of this invention corresponds to, rather than is identical to, the sequence of a recited peptide where one or more changes are made and it retains the ability to function as an antagonist of the invention in one or more of the assays as defined herein.

Antagonists of MMP-9 can also be small organic molecules, such as those natural products, or those compounds synthesized by conventional organic synthesis or combinatorial organic synthesis. Compounds can be tested for their ability to modify protein-protein interactions involving certain amino acid sequences within MMP-9 and/or β1 integrin. Compounds also are selected for reduced affinity for the control peptide AAAA.

Assay methods, as described herein, including ELISA, well known to those of skill in the art can be used to evaluate candidate antagonists for their ability to bind both FRIP-1 and the AAAA control peptide, and furthermore can be evaluated for their potency in inhibiting angiogenesis in a tissue.

Integrins

Many physiological processes require that cells come into close contact with other cells and/or extracellular matrix. Such adhesion events may be required for cell activation, migration, proliferation and differentiation. Cell-cell and cell-matrix interactions are mediated through several families of cell adhesion molecules (CAMS) including the selectins, integrins, cadherins and immunoglobulins. CAMS play an essential role in both normal and pathophysiological processes. Therefore, the targeting of specific and relevant CAMS in certain disease conditions without interfering with normal cellular functions is essential for an effective and safe therapeutic agent that inhibits cell-cell and cell-matrix interactions.

Of the various Cams discussed above, the integrin superfamily is found in various combinations on nearly every mammalian cell type. (for reviews see: E. C. Butcher, Cell, 67, 1033 (1991); T. A. Springer, Cell, 76,301 (1994); D. Cox et al., “The Pharmacology of the Integrins.” Medicinal Research Rev. 14, 195 (1994) and V. W. Engleman et al., “Cell Adhesion Integrins as Pharmaceutical Targets.” in Ann. Repts. in Medicinal Chemistry, Vol. 31, J. A. Bristol, Ed.; Acad. Press, NY, 1996, p. 19 1).

The connection between the ECM (native and degraded) and vascular cells is mediated by integrins (Brooks, P. C. (1996) European Journal of Cancer; Brooks, P. C. (1996) Cancer & Metastasis Reviews; Luscinskas & Lawler (1994)). Integrins are a family of cell surface heterodimers composed of α and β chains that mediate cellular interactions with both ECM components and other cells. Studies have provided evidence that integrins play an important role in the regulation of vascular cell adhesion and migration. Whereas the native ECM interacts with the vascular cells through one set of integrins, there is evidence that the degraded ECM interacts with a distinct (but overlapping) set of integrins. The alteration of integrin interactions upon proteolytic remodeling of the ECM is likely to result in altered cellular signaling, promoting proliferation and migration of the vascular cells.

The integrins represent one of the best characterized superfamilies of adhesion receptors. Integrins are glycoprotein heterodimers which contain a non-covalently associated alpha (a) and beta (β) subunit. Integrin subunits are transmembrane proteins which contain an extracellular domain for interacting with an extracellular matrix or cellular component, a transmembrane domain spanning the cell membrane and a cytoplasmic domain for interacting with one or more cytoskeletal components.

There are fourteen known α subunits and eight known β subunits which can pair to form at least twenty different integrin molecules. Several distinct integrin at chains are capable of pairing with one type of β chain to form a β chain subfamily.

Of particular interest here is the β₁ (β1) subfamily, which includes seven members (also known as the VLA proteins: α1β1-α7β1).

Angiogenesis and Diseases Potentially Treated by Inhibitors of Angiogenesis

As used herein, the terms “angiogenesis inhibitory,” “angiogenesis inhibiting” or “anti-angiogenic” include vasculogenesis, and are intended to mean effecting a decrease in the extent, amount, or rate of neovascularization. Effecting a decrease in the extent, amount, or rate of endothelial cell proliferation or migration in the tissue is a specific example of inhibiting angiogenesis.

The term “angiogenesis inhibitory composition” refers to a composition which inhibits angiogenesis-mediated processes such as endothelial cell migration, proliferation, tube formation and subsequently leading to the inhibition of the generation of new blood vessels from existing ones, and consequently affects angiogenesis-dependent conditions.

As used herein, the term “angiogenesis-dependent condition” is intended to mean a condition where the process of angiogenesis or vasculogenesis sustains or augments a pathological condition or beneficially influences normal physiological processes. Therefore, treatment of an angiogenesis-dependent condition in which angiogenesis sustains a pathological condition could result in mitigation of disease, while treatment of an angiogenesis-dependent condition in which angiogenesis beneficially influences normal physiological processes could result in, e.g., enhancement of a normal process.

Angiogenesis is the formation of new blood vessels from pre-existing capillaries or post-capillary venules. Vasculogenesis results from the formation of new blood vessels arising from angioblasts which are endothelial cell precursors. Both processes result in new blood vessel formation and are included in the meaning of the term angiogenesis-dependent conditions. The term “angiogenesis” as used herein is intended to include de novo formation of vessels such as that arising from vasculogenesis as well as those arising from branching and sprouting of existing vessels, capillaries and venules.

Examples of diseases in which angiogenesis plays a role in the maintenance or progression of the pathological state are listed herein in the Background of the Invention. Other diseases are known to those skilled in the art and are similarly intended to be included within the meaning of “angiogenesis-dependent condition” and similar terms as used herein.

Cancers, Tumors, and Tissues

The methods of the invention are contemplated for use in treatment of a tumor tissue of a patient with a tumor, solid tumor, a metastasis, a cancer, a melanoma, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include, but are not limited to, tumors of the skin, melanoma, lung, pancreas, breast, colon, laryngeal, ovarian, prostate, colorectal, head, neck, testicular, lymphoid, marrow, bone, sarcoma, renal, sweat gland, and the like tissues. Further examples of cancers treated are glioblastomas.

A tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

Thus, methods which inhibit angiogenesis in a diseased tissue ameliorate symptoms of the disease and, depending upon the disease, can contribute to cure of the disease. In embodiments, the invention contemplates inhibition of angiogenesis in a tissue. The extent of angiogenesis in a tissue, and therefore the extent of inhibition achieved by the present methods, can be evaluated by a variety of methods, such as are described herein.

Any of a variety of tissues, or organs comprised of organized tissues, can support angiogenesis in disease conditions including skin, muscle, gut, connective tissue, joints, bones and the like tissue in which blood vessels can invade upon angiogenic stimuli. Thus, in one embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In the absence of neovascularization of tumor tissue, the tumor tissue does not obtain the required nutrients, slows in growth, ceases additional growth, regresses and ultimately becomes necrotic resulting in killing of the tumor. The present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because their formation requires vascularization of a primary tumor so that the metastatic cancer cells can exit the primary tumor and their establishment in a secondary site requires neovascularization to support growth of the metastases.

The invention also contemplates the practice of the method in conjunction with other therapies such as conventional chemotherapy directed against solid tumors and for control of establishment of metastases. The administration of an angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases. Patients

The invention contemplates treatment of patients including human and non-human patients. In a preferred embodiment, the patient is a human. The term patient as used in the present application refers to all different types of mammals including humans and the present invention is effective with respect to all such mammals. “Patient” and “subject” are used interchangeably herein. The present invention is effective in treating any mammalian species. The present invention has particular application to agricultural and domestic mammalian species including, but not limited to, horses, primates (e.g., gorillas, monkeys, chimpanzees), cows and bulls, dogs, cats, sheep, pigs, poultry (e.g., chickens, turkeys), rodents (e.g., mice, rats), or any other veterinary animal for which the invention has uses.

It will be appreciated that a “patient suffering from cancer” of the invention may not yet be symptomatic for the disease. In one non-limiting example, where the cancer is colon cancer (which is associated with the mutant K-ras protein), a patient with a mutant K-ras protein in some cells of the colon is a patient according to the invention even though that patient may not yet be symptomatic for colon cancer. “Associated with a mutant protein” means signs or symptoms of illness in a majority of patients are present when the mutant protein is present in the patient's body, but in which signs or symptoms of illness are absent when the mutant protein is absent from the patient's body. “Signs or symptoms of illness” are clinically recognized manifestations or indications of disease.

“Administering” is defined herein as a means providing the composition to the patient in a manner that results in the composition being inside the patient's body. Such an administration can be by any route including, without limitation, locally, regionally or systemically by subcutaneous, intradermal, intravenous, intra-arterial, intraperitoneal, or intramuscular administration (e.g., injection). “Concurrent Administration” means administration within a relatively short time period from each other. Preferably such time period is less than 2 weeks, more preferably less than 7 days, most preferably less than 1 day and could even be administered simultaneously.

“Contacting” is defined herein as a means of bringing a composition as provided herein in physical proximity with a cell, organ, tissue or fluid as described herein. Contacting encompasses systemic or local administration of any of the compositions provided herein and includes, without limitation, in vitro, in vivo and/or ex vivo procedures and methods. “Combining” and “contacting” are used interchangeably herein and are meant to be defined in the same way.

The present invention is effective in treating any mammalian species which have a disease associated with angiogenesis or which reduction of angiogenesis would result in treatment of a condition including tumor metastasis, tumor growth, cell adhesion, cell proliferation or cell migration.

Modes of Carrying Out the Invention

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

As used herein, the term “angiogenesis-dependent condition” is intended to mean a condition where the process of angiogenesis or vasculogenesis sustains or augments a pathological condition, or beneficially influences normal physiological processes. Therefore, influencing processes dependent on angiogenesis to treat an angiogenesis-dependent condition could result in mitigation of the condition if angiogenesis sustains that pathological condition, while treatment of an angiogenesis-dependent condition in which angiogenesis beneficially influences normal physiological processes could result in, e.g., enhancement of a normal process.

Antibody Antagonists

Antagonists to denatured ECM components include denatured ECM component antagonists in the form of antibodies which bind to a denatured ECM component or components but bind to a native ECM component or components with a substantially reduced affinity.

Antibody antagonists to protein targets, e.g., those listed in Tables I-XVI are also contemplated for use in the methods of the invention.

Antibodies useful in the invention can be monoclonal or polyclonal. In one embodiment, antibodies used are monoclonal. A monoclonal antibody of this invention comprises antibody molecules that immunoreact with a denatured ECM component, but immunoreact with a substantially reduced affinity with the native form of the ECM component.

Monoclonal antibodies which preferentially bind to denatured collagen include monoclonal antibodies having the immunoreaction characteristics of, e.g., Mab, HUIV26, HUI77, D93 and H8.

Antibody antagonists of the invention can be generated according to a number of methods known to one of skill in the art. For example, an animal can be immunized with a denatured collagen or fragment thereof. Antibodies thus generated can be selected both for their ability to bind to denatured or proteolyzed ECM components and for a substantially reduced affinity for the native form of the same ECM component. Antibodies can, for example, be generated by the method of “subtractive immunization” (see, e.g., Brooks, P. C. et al., J. Cell. Biol. 1993, 122:1351-1359 and U.S. Publication No. 2003/0113331).

The subtractive immunization technique allows one to experimentally manipulate the immune response within mice to selectively enhance an immune response to a rare and/or low abundant epitope within a mixture of common highly antigenic epitopes. As described in U.S. Publication No. 2003/0113331 with regard to preparation of antibodies that selectively bind to denatured collagen, the method can be carried out using an ECM component as follows: mice are injected intraperitoneally with a native ECM component. At 24 and 48 hours following the injections of the native ECM component, the mice are injected with the tolerizing agent, cyclophosphamide, to kill activated B-cells that would produce antibodies directed to common immunodominant epitopes within the native ECM component. Following the tolerization protocol, the mice are next injected with thermally denatured human ECM component to stimulate an immune response to epitopes exposed following thermal denaturation. The ECM component can be denatured, e.g., by boiling for 15 minutes or by proteolysis as described herein. The injections of the thermally denatured ECM component are given every three weeks for a total of 4 to 5 injections. Serum from each mouse is tested for immunoreactivity with both the native and denatured ECM components. The mice demonstrating the highest titer for reactivity to the denatured ECM component as compared to the native ECM component are used for the production of hybridomas. Spleen cells from the selected mice are fused with myeloma cells by standard techniques. Individual hybridoma clones are tested for the production of antibody to either native or denatured ECM component. Hybridoma clones are selected that produce antibodies that demonstrate a selective reactivity to the denatured ECM component as compared to the native ECM component.

Mabs are purified by standard techniques. Monoclonal antibodies demonstrating preferential reactivity for denatured ECM proteins or other protein targets can be isotyped according to established procedures utilizing the Mouse Typer Sub-Isotyping Kit (Bio-Rad) according to the manufacturer's protocol.

IgG monoclonal antibodies can be purified from culture media or ascites by affinity to Protein-A-Agarose using standard techniques. IgM monoclonal antibodies can be purified by affinity to Mannin-Binding-Column (Pierce) using the protocol provided by the manufacturer.

As cyclophosphamide is an immunosuppressant, it may suppress B-cell proliferation so that few viable B-cells remain. If this occurs, the dosage of cyclophosphamide can be reduced to a lower level (e.g. 100 mg/kg). Alternate approaches, such as immunization with specific peptides that are suspected to play a role in regulating angiogenesis, may be used to identify antibodies that recognize cryptic sites.

As used in this application, the term “antibody” or “antibody molecule” refers to a population of a immunoglobulin molecules and/or immunological active portions of those particular immunoglobulin molecules that contain the portion of an antibody which binds to its antigens, also known as the “antibody-combining site.”

The term “antibody” also includes molecules which have been engineered through the use of molecular biological technique to include only portions of the native molecule as long as those molecules have the ability to bind to a particular antigen with the required specification. Such alternative antibody molecules include classically known portions of the antibodies molecules and single chain antibodies.

Antibodies for use in the present invention are intact immunoglobulin molecules, substantially intact immunoglobulin molecules and those portions of an immunoglobulin molecule that contain the paratope, including those portions known in the art. An antibody or functional fragment thereof, includes, but is not limited to, a polyclonal antibody, a monoclonal antibody, a chimeric antibody, a humanized antibody, a human antibody, a labeled antibody, a Fab, a F (ab)₂, a F(ab′)₂, a scFv, a genetically engineered antibody and a single chain binding polypeptide.

In one exemplary embodiment, the invention contemplates a single chain binding polypeptide having a heavy chain variable region, a light chain variable region and, optionally, an immunoglobulin Fc region. Such a molecule is a single chain variable fragment optionally having effector function through the presence of the immunoglobulin Fc region. Methods of preparing single chain binding polypeptides are known in the art (e.g., US. Patent Application 2005/0238646).

The invention embodies a truncated immunoglobulin molecule comprising a Fab fragment derived from a monoclonal antibody of this invention. The Fab fragment, lacking Fc receptor, is soluble, and affords therapeutic advantages in serum half life and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by proteolysis using papain and pepsin, respectively, on substantially intact antibodies by methods that are well known. See for example, U.S. Pat. No. 4,342,566 to Theofilopolous and Dixon. Fab′ antibody portions also are well known and are produced from F(ab′)₂ portions, followed by reduction of disulfide bonds linking the two heavy chains as with mercaptoethanol, and followed by alkylation of the resulting protein mercaptan with a reagent such as iodoacetamide.

The term monoclonal antibody as used herein refers to an antibody molecule population that has only one particular antibody combining site and is capable of immunoreacting with a particular epitope. A monoclonal antibody typically displays a single binding affinity for that epitope and such binding can be measured by standard amino acids. Monoclonal antibodies that are useful in this invention may also contain a number of different antibody combining sites wherein each antibody combining site is specific for a particular epitope. Examples of such monoclonal antibodies include bi-specific monoclonal antibodies. Monoclonal antibodies contemplated by the present invention also include monoclonal antibodies that are produced by various methods including traditional monoclonal antibodies technology and modern molecular techniques which isolate the antibody combining site of a particular antibody and express it as either a part of a immunological molecule or as part of another molecule.

A monoclonal antibody can be composed of antibodies produced by clones of a single cell called a hybridoma that produces only one kind of antibody molecule. The hybridoma cell is formed by fusing an antibody-producing cell and a myeloma or other self-perpetuating cell line. The preparation of such antibodies was first described by Kohler and Milstein, Nature 1975, 256: 495-497. Additional methods are described by Zola, Monoclonal Antibodies: A Manual of Techniques, CRC Press, Inc. (1987).

A monoclonal hybridoma culture is initiated comprising a nutrient medium containing a hybridoma that secretes antibody molecules of the appropriate specificity. The culture is maintained under conditions and for a time period sufficient for the hybridoma to secrete the antibody molecules into the medium. The hybridoma supernatant so prepared can be screened for the presence of antibody molecules that immunoreact with cryptic epitopes of ECM components.

To form the hybridoma from which the monoclonal antibody is produced, a myeloma or other self-perpetuating cell line is fused with lymphocytes obtained from the spleen of a mammal hyperimmunized with a source of a target protein.

It is preferred that the myeloma cell line used to prepare a hybridoma be from the same species as the lymphocytes. A mouse of the strain 129 G1X⁺ is typically the preferred mammal. Suitable mouse myelomas for use in the present invention include the hypoxanthine-aminopterin-thymidine-sensitive (HAT) cell lines P3×63-Ag8.653, and Sp2/0-Ag14 that are available from the American Type Culture Collection, Rockville, Md., under the designations CRL 1580 and CRL 1581, respectively.

Splenocytes are typically fused with myeloma cells using a space inhibitor such as polyethylene glycol (PEG) 1500. Fused hybrids are selected by their sensitivity to a selective growth medium, such as HAT (hypoxanthine aminopterin thymidine) medium. Hybridomas producing a monoclonal antibody of this invention can be identified using the enzyme linked immunosorbent assay (ELISA).

Media useful for the preparation of these compositions are both well known in the art and commercially available and include synthetic culture media, media derived from inbred mice and the like. An exemplary synthetic medium is Dulbecco's minimal essential medium (DMEM; Dulbecco et al., Virol. 1959, 8:396, 1959) supplemented with 4.5 g/L glucose, 20 nM glutamine, and 20% fetal calf serum. An exemplary inbred mouse strain is the BALB/c.

Alternatively, the monoclonal antibody may be produced using cloning methods to isolate the gene(s) encoding the monoclonal antibody. Such techniques are well known in the art. See, for example, the method of isolating monoclonal antibodies from an immunological repertoire as described by Sastry et al., Proc. Natl. Acad. Sci. USA 1989, 86:5728-5732; and Huse et al., Science 1989, 246:1275-1281.

Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Humanized monoclonal antibodies offer particular advantages over murine monoclonal antibodies, particularly insofar as they can be used therapeutically in humans.

Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention.

Thus, the invention contemplates, in one embodiment, a monoclonal antibody of this invention that is humanized by grafting to introduce components of the human immune system without substantially interfering with the ability of the antibody to bind antigen.

The antibody of the invention can also be a fully human antibody such as those generated, for example, by selection from an antibody phage display library displaying human single chain or double chain antibodies such as those described in de Haard, H. J. et al. (1999) J. Biol. Chem. 274:18218-30 and in Winter, G. et al. (1994) Annu. Rev. Immunol. 12:433-55.

As used herein, the term “CDR” or “complementarity determining region” is intended to mean the non-contiguous antigen combining sites found within the variable region of both heavy and light chain polypeptides. There are three CDRs in each of the heavy and light chains, i.e., six CDRs in total. These particular regions have been described by Kabat et al., J. Biol. Chem. 252: 6609-6616 (1977); Kabat et al., U.S. Dept. of Health and Human Services, “Sequences of proteins of immunological interest” (1991); by Chothia et al., J. Mol. Biol. 196: 901-917 (1987); and by MacCallum et al., J. Mol. Biol. 262: 732-745 (1996), where the definitions include overlapping or subsets of amino acid residues when compared against each other.

Peptide and Polypeptide Antagonists

Preparation of Peptide and Polypeptide Antagonists

Peptides can be linear or cyclic, although particularly preferred peptides are cyclic. Longer polypeptides, e.g., of greater than about 100 residues, can be provided in the form of a fusion protein or protein fragment. Antagonists of native or denatured ECM components also can be polypeptides or peptides. The term polypeptide refers to a sequence of 3 or more amino acids connected to one another by peptide bonds between the alpha-amino group and carboxy group of contiguous amino acid residues. The term peptide as used herein refers to a series of two or more amino acid residues connected to one to the other as in a polypeptide.

It should be understood that a subject polypeptide need not be identical to the amino acid residue sequence of a cryptic epitope of an ECM component or to an epitope of a target protein.

A subject polypeptide includes any analog, fragment or chemical derivative of a polypeptide antagonist of a target protein. Therefore, a present polypeptide can be subject to various changes, substitutions, insertions, and deletions where such changes provide for certain advantages in its use. In this regard, an antagonist polypeptide of this invention corresponds to, rather than is identical to, the sequence of a recited peptide where one or more changes are made and it retains the ability to function as an antagonist in one or more of the assays as defined herein.

The polypeptides or peptides of the present invention may be a peptides or polypeptides derivative that include those residue or chemical changes including amides, conjugates with proteins, cyclic peptides, polymerized peptides and analogs of fragments of chemically modified peptides or proteins and other types of derivatives.

The term “analog” includes any polypeptide having an amino acid residue sequence substantially identical to a given sequence. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” as used in this application includes chemically derivatized residues that are used to replace a non-derivatized residue in a peptide or polypeptide that results in a peptide or polypeptide that maintains the desired function.

Polypeptide antagonists of the present invention can have sequences in which one or more conservative or non-conservative substitutions have been made, usually up to about 30 (number) percent. Up to about 10 (number) percent of the amino acid residues can be substituted. Additional residues may also be added at either terminus of a polypeptide for the purpose of providing a “linker” by which the polypeptides of this invention can be conveniently affixed to a label or solid matrix, or carrier.

The term “chemical derivative” as used in this application refers to polypeptide or peptide having amino acid sequence residues that are changed or derivatized chemically by using a reaction with a functional side group. Other contemplated derivitizations of peptides or polypeptides includes a chemical derivative which uses backbone modifications including α-amino acids substitutions, such as N-methyl, N-ethyl, N-propyl and other similar substitutions to replace various residues within the backbone. Other potential derivatives utilizing backbone modifications include α-carbonyl substitutions such as thioester, thioamide, guanidino, and other similar substitutions. The present invention also contemplates the use of derivatized molecules which include pre-amino acid groups which have been derivatized to form hydrochlorides, p-toleune sulfonyl groups carbobenzoxy groups, t-butyloxycarbonyl groups, chloroacetyl groups or formyl groups. The free carboxyl groups typically may be derivatized to form salts, methyl and ethyl esters or other types of esters or hydrazides. In the free hydroxyl groups may be derivatized to form O-acyl or O-alkyl derivatives of those peptides or polypeptides.

Labels, solid matrices and carriers that can be used with the polypeptides of this invention are described herein below.

Amino acid residue linkers are usually at least one residue and can be 40 or more residues, more often 1 to 10 residues, but do not form an epitope of a target protein, i.e. a cryptic epitope of an ECM component. Typical amino acid residues used for linking are tyrosine, cysteine, lysine, glutamic and aspartic acid, or the like. In addition, a subject polypeptide can differ, unless otherwise specified, from the natural sequence of the ECM cryptic epitope ligand by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong the half-life of the polypeptides in solutions, particularly biological fluids where proteases may be present. In this regard, polypeptide cyclization is also a useful terminal modification because of the stable structures formed by cyclization and in view of the biological activities observed for such cyclic peptides.

Any peptide of the present invention may be used in the form of a pharmaceutically acceptable salt. Suitable acids which are capable of forming salts with the peptides of the present invention include inorganic acids such as trifluoroacetic acid (TFA) hydrochloric acid (HC), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HC and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine) and optionally substituted ethanolamines (e.g. ethanolamine and diethanolamine).

In addition, a peptide useful in the methods of this invention can be prepared without including a free ionic salt in which the charged acid or base groups present in the amino acid residue side groups (e.g., Arg, Asp, and the like) associate and neutralize each other to form an “inner salt” compound.

A peptide of the present invention can be synthesized by any of the techniques that are known to those skilled in the polypeptide art, including recombinant DNA techniques. Synthetic chemistry techniques, such as a solid-phase Merrifield-type synthesis can be advantageous for reasons of purity, antigenic specificity, freedom from undesired side products, ease of production and the like. Summaries of the many techniques available can be found in, e.g., Steward et al., “Solid Phase Peptide Synthesis,” W.H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis, and Schroder et al., “The Peptides,” Vol. 1, Academic Press (New York), 1965 (for classical solution synthesis). Appropriate protective groups usable in such synthesis are also described in J. F. W. McOmie, “Protective Groups in Organic Chemistry,” Plenum Press, New York, 1973.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

Linear polypeptides may be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Alternative methods for cyclic peptide synthesis are described by Gurrath et al., Eur. J. Biochem., 210:911-921 (1992).

In addition, the antagonist can be provided in the form of a fusion protein. Fusion proteins are proteins produced by recombinant DNA methods known and described in the art, in which the subject polypeptide is expressed as a fusion with a second carrier protein such as a glutathione sulfhydryl transferase (GST) or other well-known carrier.

Thus, a polypeptide can be present in any of a variety of forms of peptide derivatives, including amides, conjugates with proteins, cyclized peptides, polymerized peptides, analogs, fragments, chemically modified peptides, and like derivatives.

Identification of Peptide and Polypeptide Antagonists

Contemplated herein is the use of antagonists in the form of polypeptides. A polypeptide antagonist of a target protein can be any peptide or polypeptide capable of binding to a target protein, but one that binds to the native form of the target protein with substantially reduced affinity.

The identification of denatured ECM component antagonist peptides having selectivity for denatured ECM components or antagonist peptides specific for other target proteins can readily be identified in a typical inhibition of binding assay, such as the ELISA assay.

Peptide and polypeptide antagonists of denatured ECM components can be generated by a number of techniques known to one of skill in the art. For example, a two-hybrid system (e.g., Fields, S., Nature 1989, 340:245-6) can use a protein fragment, e.g., a fragment of denatured collagen or laminin, as “bait” for selecting protein antagonists from a library that bind to the fragment. The library of potential antagonists can be derived from a cDNA library, for example. The potential antagonists can also be variants of known target protein binding proteins. Such proteins can be randomly mutagenized or subjected to gene shuffling, or other available techniques for generating sequence diversity.

Peptide and polypeptide antagonists also can be identified by techniques of molecular evolution. Libraries of proteins can be generated by mutagenesis, gene shuffling or other available techniques for generating molecular diversity. Protein pools representing numerous variants can be selected for their ability to bind to denatured target protein, for instance by passing such protein pools over a solid matrix to which a target protein, e.g., denatured collagen, has been attached. Elution with gradients of salt, for example, can provide purification of variants with affinity for the target protein. When screening for denatured ECM protein antagonists peptides, a negative selection step also can be included whereby such pools are passed over a solid matrix to which a native ECM component has been attached. The filtrate will contain those variants within the pool that have a reduced affinity for the native form of the collagen. This method can be applied to the identification of antagonists having specificity for the denatured forms of other ECM components.

Peptide and polypeptide antagonists of the invention also can be generated by phage display. A randomized peptide or protein can be expressed on the surface of a phagemid particle as a fusion with a phage coat protein. Techniques of monovalent phage display are widely available (see, e.g., Lowman H. B. et al., Biochemistry 1991, 30:10832-8.) For example, phage expressing randomized peptide or protein libraries can be panned with a solid matrix to which a native ECM component molecule has been attached. Remaining phage do not bind the native molecule, or bind native molecules with substantially reduced affinity. The phage are then panned against a solid matrix to which the denatured ECM component has been attached. Bound phage are isolated and separated from the solid matrix by either a change in solution conditions or, for a suitably designed construct, by proteolytic cleavage of a linker region connecting the phage coat protein with the randomized peptide or protein library. The isolated phage can be sequenced to determine the identity of the selected antagonist.

In another embodiment, a polypeptide includes any analog, fragment or chemical derivative of a given polypeptide so long as the polypeptide is an antagonist of a target protein. Therefore, a present polypeptide can be subject to various changes, substitutions, insertions, and deletions where such changes provide for certain advantages in its use. In this regard, an antagonist polypeptide of this invention corresponds to, rather than is identical to, the sequence of a recited peptide where one or more changes are made and it retains the ability to function as a target protein antagonist.

Other Antagonists

Antagonists as provided herein also can be small organic molecules, such as those natural products, or those compounds synthesized by conventional organic synthesis or combinatorial organic synthesis. Compounds can be tested for their ability to bind to a target protein for example by using the affinity-purification technique described herein.

Compounds also are selected for reduced affinity for the native form of the target protein by a similar affinity-purification technique.

Antagonists as provided herein also can be non-peptidic compounds, including, for example, oligonucleotides. Oligonucleotides, as used herein, refers to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g. phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. “Oligonucleotide” also encompasses heteropolymers with distinct structures that also present aromatic bases, including polyamide nucleic acids and the like.

An oligonucleotide antagonist can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution containing the pool over a solid matrix to which a target protein or fragment thereof has been affixed. Sequences within the pool that bind to the target protein are retained on the solid matrix. These sequences are eluted with a solution of different salt concentration or pH. Sequences selected are subjected to a second selection step. For example, when screening for antagonists of denatured ECM components, the selected pool is passed over a second solid matrix to which the native ECM component has been affixed. The column retains those sequences that bind to the native ECM component, thus enriching the pool for sequences specific for the denatured ECM component. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

Identification of Antagonists of Cryptic Epitopes of ECM Components

Potentially useful antagonists of cryptic epitopes of ECM components have been described in U.S. Publication No. 2003/0113331; U.S. Publication No. 2004/0242490 A1; WO 2004/073649; U.S. Publication No. 2004/224896 A1, and; WO 2004/087734. In the identification methods of the invention, candidate antagonists are evaluated for their ability to bind to denatured ECM components, and furthermore can be evaluated for their potency in altering metastasis, angiogenesis, and other tumor development processes, in a tissue. Measurement of binding of antagonists to denatured or native ECM components in the solid phase can be accomplished, e.g., using an enzyme-linked-immunosorbent assay (ELISA), described in these publications and herein. The ELISA is commonly used and well-known to those of skill in the art.

The ELISA also can be used to identify compounds which exhibit increased specificity for denatured, as compared to the native forms of ECM components. The specificity assay is conducted by running parallel ELISAs in which a potential antagonist is screened concurrently in separate assay chambers for the ability to bind denatured and native ECM components. Another technique for measuring apparent binding affinity familiar to those of skill in the art is a surface plasmon resonance technique (analyzed on a BIACORE 2000 system) (Liljeblad, et al., Glyco. J. 2000, 17: 323-329). Standard measurements and traditional binding assays are described by Heeley, R. P., Endocr. Res. 2002, 28: 217-229.

Antagonists of denatured ECM components can also be identified by their ability to compete for binding with antagonists useful in the present invention. For example, putative antagonists can be screened by monitoring their effect on the affinity of a known antagonist, such as antibody HUIV26, described in U.S. Publication No. 2003/0113331. Such antagonists likely have the same specificity as, and recognize the same cryptic epitope, as the antibodies themselves. Putative antagonists selected by such a screening method can bind either to the ECM component or to the antagonist. Antagonists can be selected from the putative antagonists by conventional binding assays to determine those that bind to the cryptic epitope of the ECM component but not to the known antagonist.

Antagonists can be identified by their ability to bind to a solid matrix containing a denatured ECM component. Such putative antagonists are collected after altering solution conditions, such as salt concentration, pH, temperature, etc. The putative antagonists are further identified by their ability to pass through, under appropriate solution conditions, a solid matrix to which a native ECM component has been affixed.

Antagonists useful herein can be assayed for their ability to influence tumor development processes, e.g., angiogenesis, tumor metastasis, cell adhesion, cell migration, and tumor growth in a tissue as well as their effect on angiogenesis-dependent conditions. Any suitable assay known to one of skill in the art can be used to monitor such effects. Several such assays are described herein.

In a related embodiment, the invention contemplates the practice of the method in conjunction with other therapies such as conventional chemotherapy directed against solid tumors and for control of establishment of metastases.

Other Cancer Therapies

Other cancer therapies useful in the combinations provided herein include but are not limited to chemotherapy, radiation therapy, biological therapies, hormonal therapies and/or surgery, or combinations thereof. For example, combinations could include one or more doses of a prophylactically or therapeutically effective amount of one or more biological therapies/immunotherapies or hormonal therapies other than the antagonists of angiogenesis. Such methods can optionally further comprise the administration of one or more doses of a prophylactically or therapeutically effective amount of other cancer therapies, such as but not limited to radiation therapy, chemotherapies, and/or surgery. Examples of such biological therapies/immunotherapies include, but are not limited to, tamoxifen, leuprolide or other LHRH agonists, non-steroidal antiandrogens (flutamide, nilutamide, bicalutamide), steroidal antiandrogens (cyproterone acetate), estrogens (DES, chlorotrianisene, ethinyl estradiol, conjugated estrogens U.S.P., DES-diphosphate), aminoglutethimide, hydrocortisone, flutamide withdrawal, progesterone, ketoconazole, prednisone, interferon-alpha, interferon-beta, interferon-gamma, interleukin-2, tumor necrosis factor-alpha, vincristine, doxorubicin, heceptin, uinblastin and melphalan. Biological therapies also include cytokines such as, but not limited to, TNF ligand family members such as TRAIL anti-cancer agonists that induce apoptosis, TRAIL antibodies that bind to TRAIL receptors 1 and 2 otherwise known as DR4 and DR5 (Death Domain Containing Receptors 4 and 5), as well as DR4 and DR5. TRAIL and TRAIL antibodies, ligands and receptors are known in the art and described in U.S. Pat. Nos. 6,342,363, 6,284,236, 6,072,047 and 5,763,223.

Chemotherapeutic Agents

It is recognized in the art that a benefit of using chemotherapeutic agents in conjunction with other therapies, e.g., the antagonists of the present invention, can be useful for allowing administration of lower doses of chemotherapeutic agents, thereby potentially resulting in a reduction in toxic side effects. It is also known to those of skill in the art that therapeutically-effective dosages of chemotherapeutic agents can vary when these drugs are used in treatment combinations. Methods for experimentally determining therapeutically-effective dosages of chemotherapeutic drugs and other agents for use in combination treatment regimens are described in the literature. For example, the use of metronomic chemotherapy dosing, i.e., providing more frequent, lower doses in order to minimize toxic side effects, has been described extensively in the literature. A combination treatment regimen encompasses treatment regimens in which administration of a chemotherapeutic agent is initiated prior to, during, or after treatment with the second agent, e.g., an antibody, and continues until any time during treatment with the other agent or after termination of treatment with the other agent. It also includes treatments in which the agents being used in combination are administered simultaneously or at different times and/or at decreasing or increasing intervals during the treatment period. Combination treatment includes periodic treatments that start and stop at various times to assist with the clinical management of the patient. For example, an agent in the combination can be administered weekly at the onset of treatment, decreasing to biweekly, and decreasing further as appropriate.

Commonly used chemotherapeutic agents, and the preparation of such agents, are described, e.g., in U.S. Pat. No. 6,858,598, hereby incorporated by reference in its entirety. Chemotherapeutic agents useful in the methods of the present invention include, e.g., taxanes (i.e., taxol, docetaxel, paclitaxel), campthothecin (i.e., irinotecan, or CPT-11), dacarbazine (DTIC), adriamycin, bleomycin, gemcitabine, cyclophosphamide, oxaliplatin, fludarabine, cisplatin and carboplatin.

The phrase “taxane” includes a family of diterpene alkaloids all of which contain a particular eight (8) member “taxane” ring structure. Taxanes, such as paclitaxel, prevent the normal post-division breakdown of microtubules, which form to pull and separate the newly duplicated chromosome pairs to opposite poles of a cell prior to cell division. In rapidly dividing cancer cells, taxane therapy causes the microtubules to accumulate, ultimately preventing further division of the cancer cell. Taxane therapy also affects other cell processes dependent on microtubules, including cell motility, cell shape and intracellular transport. Certain adverse side-effects associated with taxane therapy can be classified into cardiac effects, neurotoxicity, haematological toxicity, and hypersensitivity reactions. (See Exp. Opin. Thera. Patents (1998) 8(5)). Specific adverse side-effects include neutropenia, alopecia, bradycardia, cardiac conduction defects, acute hypersensitivity reactions, neuropathy, mucositis, dermatitis, extravascular fluid accumulation, arthralgias, and myalgias. Various treatment regimens have been developed in an effort to minimize the side effects of taxane therapy, but adverse side-effects remain the limiting factor in taxane therapy.

Taxane derivatives have been found to be useful in treating refractory ovarian carcinoma, urothelial cancer, breast carcinoma, melanoma, non-small-cell lung carcinoma, gastric, and colon carcinomas, squamous carcinoma of the head and neck, lymphoblastic and myeloblastic leukemia, and carcinoma of the esophagus.

Gemcitabine is a pyrimidine antimetabolite with antineoplastic activity against a wide range of solid tumors including metastatic pancreatic carcinoma, non-small cell lung cancer, ovarian and breast cancer. It is sold under the trademark, GEMZAR® by Eli Lilly and Company, Indianapolis, Ind. Its use and production are known in the art and are described, e.g., in U.S. Pat. Nos. 5,464,826, 5,912,366, and 6,001,994, incorporated herein by reference in their entirety, as well as in Kaye, J. Clin. Oncol. 12, 1527 (1994), and in Plunkett et al., Nucleosides Nucleotides 8, 775 (1989).

Irinotecan, a derivative of camptothecin, is also called CPT-11. It is widely used as a first-line therapy for colorectal cancer. Methods for preparing and administering irinotecan are described in the art, e.g., in U.S. Pat. No. 4,604,463.

Methods for experimentally determining chemotherapeutic dosages in animal models prior to administering the drug to humans are known in the art and described in the literature. Chemotherapy doses for animals are normally expressed as mg/kg and those for humans in mg/m². The approximate conversion factor for these units is 1 mg/kg to 35 mg/m².

Single-therapy dosages in humans are described, e.g., in Cancer Management: A Multidisciplinary Approach (Medical, Surgical, and Radiation Oncology), eds. Pazdur R, Coia L R, Hoskins W J, Wagman L D (2000), Publisher PRR, Melville, N.Y., pp 984-988. For example, breast cancer in humans can be treated using taxol (or paclitaxel) at a dosage of 175 mg/m² (5 mg/kg) repeated every 34 weeks, or 80-100 mg/m2 (2.3-2.9 mg/kg) per week. Paclitaxel is typically administered in a 15420 mg/m² dose over a 6 to 24 hour infusion. For renal cell carcinoma, squamous carcinoma of head and neck, carcinoma of esophagus, small and non-small cell lung cancer, and breast cancer, paclitaxel is typically administered as a 250 mg/m² 24 hour infusion every 3 weeks. For refractory ovarian cancer paclitaxel is typically dose-escalated starting at 110 mg/m². Docetaxel is typically administered in a 60-100 mg/m² i. v. over 1 hour, every three weeks.

Colorectal adenocarcinoma in humans can be treated using irinotecan (or CPT-11) at a dosage of 125 mg/m2 (3.57 mg/kg) once/week for 4 weeks, or 350 mg/m2 (10 mg/kg) every 3 weeks. Pancreatic cancer in humans can be treated with gemcitabine at a dosage of 1000 mg/m2 (28.5 mg/kg) 1×/week for 7 weeks.

It should be noted, however, that specific dose regimen depends upon dosing considerations based upon a variety of factors including the type of neoplasia; the stage of the neoplasm; the age, weight, sex, and medical condition of the patient; the route of administration; the renal and hepatic function of the patient; and the particular agents and combination employed, etc.

The administration of angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Antagonists of Integrins

Antagonists of integrins bind to and interfere with the functional interactions of the integrins with natural integrin ligands. As used herein, the term “antagonists” refers to molecules or compounds including, but not limited to, antibodies, peptides, oligonucleotides, and small molecule compounds. Such antagonists, and methods for preparing and identifying therein, are described in e.g., U.S. Pat. No. 6,500,924; U.S. Pat. No. 5,753,230; U.S. Pub. No. 2004/0063790 A1; U.S. Pub. No. 2004/0258691; U.S. Pub. No. 2004/0265317; U.S. Pub. No. 2005/0002936, and; U.S. Pub. No. 2004/0176334 (the disclosures of which are incorporated herein by reference in their entirety) as well as in the present application.

Contemplated herein as examples of useful antagonists analogs of an integrin which are derived from the portion of the integrin that is considered to be the ligand binding site, integrin mimetics, mimetics of natural ligand of integrin that include or functionally act as the structural region involved in the integrin—ligand binding, sequences corresponding to the functional binding domain of the integrin natural ligand including peptides and polypeptides, sequences corresponding to the RGD domain of the natural ligand which bond to integrin including peptides, polypeptides and the like, and antibodies monoclonal antibodies which bind with the integrin or the natural integrin ligand.

The useful integrin antagonists of integrin have the ability to substantially inhibit the binding of a naturally occurring ligand such as vitronectin or fibrinogen to the αvβ3 molecules. At a concentration of less than 5 μm, concentrations less than 0.1 μm, and concentrations of less than 0.05 μm. The term “substantially” indicates that at least 50% of the binding of fibrinogen is reduced in a presence of the αvβ3 antagonist. The term “IC₅₀ value” as used herein is meant to refer to 50% inhibition in binding.

An integrin antagonist may potentially show selective binding to that integrin as compared to the binding to other integrins. For example, when an αvβ3 antagonist does show selectivity the binding of αvβ3 to fibrinogen is substantially inhibited but the binding between αvβ3 and other integrins, such a αvβ1, αvβ5, αvβ3 is not substantially inhibited. The αvβ3 antagonists are particularly useful in the present invention to show a 10-fold to a 100-fold lower IC₅₀ value for inhibiting the binding of αvβ3 to fibrinogen when compared to the IC₅₀ value for binding of αvβ3 to other integrins. The methods for measuring IC₅₀ activity are well known in the art and, for example, methods for demonstrating inhibitions of fibronecting to a particular integrin are now described in the United States Patent Publication No. 2004/0063790.

Peptide and Polypeptide Antagonists of Integrins

The peptides useful in the compositions and methods provided herein can be either linear or cyclical although cyclic peptides are preferred in some applications. Peptides or polypeptides are in longer length, such as a length of greater than 100 amino acid residues, can be produced as a fusion protein or a fragment of a protein as described in the description of this invention. Peptides and polypeptides that are useful in this invention may not have the identical amino acid residue sequences of the integrin's natural ligand, and it may have that amino acid sequence as part of a longer sequence or a fusion protein as long as that polypeptide or peptide is able to function as an integrin antagonist in the assays useful in this invention.

Polypeptides and peptides of the combinations and methods provided herein include any fragment, analog or chemical derivative of that peptide or polypeptide that has an amino acid residue sequence as shown in this application, and that the particular amino acid residue sequence, fragment or chemical derivative functions as an integrin antagonist. The peptides and polypeptides, as contemplated, may include changes, substitutions, insertions and deletions where the changes in the sequence or particular chemical makeup of particular residues provide for certain advantages in the present invention. An integrin antagonist polypeptide or peptide useful in the compositions and methods provided herein need not be identical to but rather may correspond to the sequence of a particular peptide or polypeptide that is recited in the present application where changes made to that polypeptide or peptide between the integrin antagonist function in an assay described herein.

The polypeptides or peptides provided herein may be a peptides or polypeptides derivative that include those residue or chemical changes including amides, conjugates with proteins, cyclic peptides, polymerized peptides and analogs of fragments of chemically modified peptides or proteins and other types of derivatives.

As used herein, the term “analog” includes peptides and polypeptides having a sequence of amino acid residues that is substantially identical to an amino acid sequence specifically described in this application in which one or more amino acids has been conservatively substituted with an amino acid residue that functions in a similar manner and allows the resulting αvβ3 antagonist to have the activity described in this application. Conservative substitutions are well known in the art and include the substitutions one non-polar (hydrophobic) residue such as isoleucine, valine, leucine or methionine for another, the substitution of a polar (hydrophilic) residue for another such as the substitution of arginine and lysine, glutamine and asparagine, lysine and serine or a substitution of a basic residue for another basic residue such as lysine, arginine or histidine substitutions. Other conservative substitutions would include the substitutions of acidic amino acid residues for another such as the substitution of aspartic acids or glutamic acid.

The term “conservative substitution” as used in this application includes chemically derivatized residues that are used to replace a non-derivatized residue in a peptide or polypeptide that results in a peptide or polypeptide that maintains the desired function.

The term “chemical derivative” as used in this application refers to polypeptide or peptide having amino acid sequence residues that are changed or derivatized chemically by using a reaction with a functional side group. Other contemplated derivitizations of peptides or polypeptides includes a chemical derivative which uses backbone modifications including α-amino acids substitutions, such as N-methyl, N-ethyl, N-propyl and other similar substitutions to replace various residues within the backbone. Other potential derivatives utilizing backbone modifications include α-carbonyl substitutions such as thioester, thioamide, guanidino, and other similar substitutions. The present invention also contemplates the use of derivatized molecules which include pre-amino acid groups which have been derivatized to form hydrochlorides, p-toleune sulfonyl groups carbobenzoxy groups, t-butyloxycarbonyl groups, chloroacetyl groups or formyl groups. The free carboxyl groups typically may be derivatized to form salts, methyl and ethyl esters or other types of esters or hydrazides. The free hydroxyl groups may be derivatized to form O-acyl or O-alkyl derivatives of those peptides or polypeptides.

Solid matrices, labels and carriers that can be used with the polypeptides of this invention are described herein below.

Amino acid residue linkers are usually at least one residue and can be 40 or more residues, more often 1 to 10 residues. Tyrosine, cysteine, lysine, glutamic and aspartic acid are some examples of amino acid residues which are typically used for linking. In addition, a subject polypeptide can differ, unless otherwise specified, from the sequence of an αvβ3 ligand by modifying the sequence with terminal-NH₂ acylation, e.g., acetylation, or thioglycolic acid amidation, by terminal-carboxylamidation, e.g., with ammonia, methylamine, and the like terminal modifications. It is well known that terminal modifications are useful to reduce susceptibility by proteinase digestion, and therefore serve to prolong the half-life of the polypeptides in solutions and in particular in biological fluids where proteases may be present. In this regard, polypeptide cyclization is also a useful terminal modification in view of the biological activities observed for such cyclic peptides and because of the stable structures formed by cyclization.

A peptide provided herein may be used in the form of a pharmaceutically acceptable salt. Suitable acids which are capable of forming salts with the peptides of the present invention include inorganic acids such as trifluoroacetic acid (TFA) hydrochloric acid (HC), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the peptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine) and optionally substituted ethanolamines (e.g. ethanolamine and diethanolamine).

In addition, a peptide useful in the methods described herein can be prepared without including a free ionic salt in which the charged acid or base groups present in the amino acid residue side groups (e.g., Arg, Asp, and the like) associate and neutralize each other to form an “inner salt” compound.

A peptide as provided herein can be synthesized by any of the techniques that are known to those skilled in the art, including polypeptide and recombinant DNA techniques. Synthetic chemistry techniques, such as a solid-phase Merrifield-type synthesis can be advantageous since they produce products having high purity, antigenic specificity, freedom from undesired side products, ease of production and the like. Summaries of the some techniques available can be found in, e.g., Steward et al., “Solid Phase Peptide Synthesis,” W.H. Freeman Co., San Francisco, 1969; Bodanszky, et al., “Peptide Synthesis,” John Wiley & Sons, Second Edition, 1976; J. Meienhofer, “Hormonal Proteins and Peptides,” Vol. 2, p. 46, Academic Press (New York), 1983; Merrifield, Adv. Enzymol. 1969, 32:221-96; Fields et al., Int. J. Peptide Protein Res. 1990, 35:161-214; U.S. Pat. No. 4,244,946 for solid phase peptide synthesis, and Schroder et al., “The Peptides,” Vol. 1, Academic Press (New York), 1965 (for classical solution synthesis). Such synthesis can utilize appropriate protective groups which are described in J. F. W. McOmie, “Protective Groups in Organic Chemistry,” Plenum Press, New York, 1973.

Solid-phase synthesis methods generally comprise the sequential addition of one or more amino acid residues or suitably protected amino acid residues to a growing peptide chain. Normally, either the amino or carboxyl group of the first amino acid residue is protected by a suitable, selectively removable protecting group. For amino acids containing a reactive side group (e.g., lysine), a different, selectively removable protecting group is utilized.

In solid phase synthesis, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next suitably protected amino acid is then added, and so forth. After all the desired amino acids have been linked in the proper sequence, any remaining terminal and side group protecting groups (and solid support) are removed sequentially or concurrently, to afford the final linear polypeptide.

Linear polypeptides may be reacted to form their corresponding cyclic peptides. A method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol, sodium hydroxide solution is added, and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Cyclic peptide synthesis can be achieved by alternative methods as described by Gurrath et al., Eur. J. Biochem. 1992, 210:911-921.

In addition, the antagonist can be provided in the form of a fusion protein. Fusion proteins are proteins produced by recombinant DNA methods known and described in the art, in which the subject polypeptide is expressed as a fusion with a second carrier protein such as a glutathione sulfhydryl transferase (GST) or other well-known carrier.

Thus, a polypeptide can be present in any of a variety of forms of peptide derivatives, including amides, conjugates with proteins, cyclized peptides, polymerized peptides, analogs, fragments, chemically modified peptides, and like derivatives.

Specific peptides and derivative αvβ3 antagonist peptides contemplated as candidates for use in the present invention, including polypeptides derived from MMP-2, are disclosed in U.S. Pub. No. 2003/0176334.

A polypeptide (peptide) integrin antagonist can have the sequence characteristics of the natural ligand of the integrin. Alternatively, the integrin antagonist can have the sequence characteristics of the integrin itself at the region involved in integrin—ligand interaction and display integrin antagonist activity as described herein. An integrin antagonist peptide can contain the RGD tripeptide and correspond in sequence to the natural ligand in the RGD-containing region.

Polypeptides can have a sequence corresponding to the amino acid sequence of the RGD-containing region of a natural ligand of αvβ3 such as fibrinogen, vitronectin, von Willebrand factor, laminin, thrombospondin, and the like. The sequences of these αvβ3 ligands are well-known. An integrin antagonist peptide can be derived from any of the natural ligands.

Antibody Antagonists of Integrins

Polyclonal or monoclonal integrin antagonists in the form of antibodies that immunoreact with the integrin and inhibit integrin binding to its natural ligand are contemplated for use in embodiments of the present invention. Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Particular monoclonal antibodies of this invention immunoreact with isolated αvβ3, and inhibit ECM component binding to αvβ3. Preferred monoclonal antibodies which preferentially bind to αvβ3 include a monoclonal antibody having the immunoreaction characteristics of Mab LM609, secreted by hybridoma cell line ATCC HB 9537. Mab LM609 has been described previously, e.g. in U.S. Publication No. 2005/0002936.

As used in this application the term “antibody” or “antibody molecule” refers to a population of a immunoglobulin molecules and/or immunological active portions of those particular immunoglobulin molecules that contain the portion of an antibody which binds to its antigens, also known as the “antibody-combining site.”

The term “antibody” also includes molecules which have been engineered through the use of molecular biological technique to include only portions of the native molecule as long as those molecules have the ability to bind to a particular antigen with the required specification. Such alternative antibody molecules include classically known portions of the antibodies molecules and single chain antibodies.

Antibodies for use in the compositions and methods provided herein are intact immunoglobulin molecules, substantially intact immunoglobulin molecules and those portions of an immunoglobulin molecule that contain the paratope, including those portions known in the art. An antibody or functional fragment thereof, includes, but is not limited to, a polyclonal antibody, a monoclonal antibody, a chimeric antibody, a humanized antibody, a human antibody, a labeled antibody, a Fab, a F (ab)₂, a F(ab′)₂, a scFv, a genetically engineered antibody and a single chain binding polypeptide.

In one exemplary embodiment, the invention contemplates a single chain binding polypeptide having a heavy chain variable region, a light chain variable region and, optionally, an immunoglobulin Fc region. Such a molecule is a single chain variable fragment optionally having effector function through the presence of the immunoglobulin Fc region. Methods of preparing single chain binding polypeptides are known in the art (e.g., US. Patent Application 2005/0238646).

Encompassed herein is a truncated immunoglobulin molecule comprising a Fab fragment derived from a monoclonal antibody of this invention. The Fab fragment, lacking Fc receptor, is soluble, and affords therapeutic advantages in serum half life and diagnostic advantages in modes of using the soluble Fab fragment. The preparation of a soluble Fab fragment is generally known in the immunological arts and can be accomplished by a variety of methods.

For example, Fab and F(ab′)₂ portions (fragments) of antibodies are prepared by proteolysis using papain and pepsin, respectively, on substantially intact antibodies by methods that are well known. See for example, U.S. Pat. No. 4,342,566 to Theofilopolous and Dixon. Fab′ antibody portions also are well known and are produced from F(ab′)₂ portions, followed by reduction of disulfide bonds linking the two heavy chains as with mercaptoethanol, and followed by alkylation of the resulting protein mercaptan with a reagent such as iodoacetamide.

The term monoclonal antibody as used herein refers to an antibody molecule population that has only one particular antibody combining site and is capable of immunoreacting with a particular epitope. A monoclonal antibody typically displays a single binding affinity for that epitope and such binding can be measured by standard amino acids. Monoclonal antibodies that are useful in this invention may also contain a number of different antibody combining sites wherein each antibody combining site is specific for a particular epitope. Examples of such monoclonal antibodies include bi-specific monoclonal antibodies. Monoclonal antibodies contemplated by the present invention also include monoclonal antibodies that are produced by various methods including traditional monoclonal antibodies technology and modern molecular techniques which isolate the antibody combining site of a particular antibody and express it as either a part of a immunological molecule or as part of another molecule.

A monoclonal antibody can be composed of antibodies produced by clones of a single cell called a hybridoma that produces only one kind of antibody molecule. The hybridoma cell is formed by fusing an antibody-producing cell and a myeloma or other self-perpetuating cell line. The preparation of such antibodies was first described by Kohler and Milstein, Nature 1975, 256:495-497. Additional methods are described by Zola, Monoclonal Antibodies: A Manual of Techniques, CRC Press, Inc. (1987).

A monoclonal hybridoma culture is initiated comprising a nutrient medium containing a hybridoma that secretes antibody molecules of the appropriate specificity. The culture is maintained under conditions and for a time period sufficient for the hybridoma to secrete the antibody molecules into the medium. The hybridoma supernatant so prepared can be screened for the presence of antibody molecules that immunoreact with αvβ3.

To form the hybridoma from which the monoclonal antibody is produced, a myeloma or other self-perpetuating cell line is fused with lymphocytes obtained from the spleen of a mammal hyperimmunized with a source of αvβ3.

It is preferred that the myeloma cell line used to prepare a hybridoma be from the same species as the lymphocytes. A mouse of the strain 129 G1X⁺ is typically the preferred mammal. Suitable mouse myelomas for use in the present invention include the hypoxanthine-aminopterin-thymidine-sensitive (HAT) cell lines P3×63-Ag8.653, and Sp2/0-Ag14 that are available from the American Type Culture Collection, Rockville, Md., under the designations CRL 1580 and CRL 1581, respectively.

Splenocytes are typically fused with myeloma cells using a space inhibitor such as polyethylene glycol (PEG) 1500. Fused hybrids are selected by their sensitivity to a selective growth medium, such as HAT (hypoxanthine aminopterin thymidine) medium. Hybridomas producing a monoclonal antibody of this invention can be identified using the enzyme linked immunosorbent assay (ELISA).

Media useful for the preparation of these compositions are both well known in the art and commercially available and include synthetic culture media, media derived from inbred mice and the like. An exemplary synthetic medium is Dulbecco's minimal essential medium (DMEM; Dulbecco et al., Virol. 1959, 8:396, 1959) supplemented with 4.5 g/L glucose, 20 nM glutamine, and 20% fetal calf serum. An exemplary inbred mouse strain is the BALB/c.

Alternatively, the monoclonal antibody may be produced using cloning methods to isolate the gene(s) encoding the monoclonal antibody. Such techniques are well known in the art. See, for example, the method of isolating monoclonal antibodies from an immunological repertoire as described by Sastry et al., Proc. Natl. Acad. Sci. USA 1989, 86:5728-5732; and Huse et al., Science 1989, 246:1275-1281.

Antibodies, whether polyclonal or monoclonal, can be raised against the desired proteins or peptides by any methods known in the art (see e.g., Antibody Production: Essential Techniques, Delves, Wiley, John & Sons, Inc., 1997; Basic Methods in Antibody Production and Characterization, Howard and Bethell, CRC Press, Inc., 1999; and Monoclonal Antibody Production Techniques and Applications: Hybridoma Techniques, Schook, Marcel Dekker, 1987).

Humanized monoclonal antibodies offer advantages over murine monoclonal antibodies, particularly insofar as they can be used therapeutically in humans. Human antibodies are not cleared from the circulation as rapidly as “foreign” antigens, and do not activate the immune system in the same manner as foreign antigens and foreign antibodies. Methods of preparing “humanized” antibodies are known in the art, and can be applied to the antibodies of the present invention.

Thus, the invention contemplates, in one embodiment, a monoclonal antibody of this invention that is humanized by grafting to introduce components of the human immune system without substantially interfering with the ability of the antibody to bind antigen.

The antibody of the invention can also be a fully human antibody such as those generated, for example, by selection from an antibody phage display library displaying human single chain or double chain antibodies such as those described in de Haard, H. J. et al., J. Biol. Chem. 1999, 274:18218-30 and in Winter, G. et al., Annu. Rev. Immunol. 1994, 12:433-55.

Other Antagonists of Integrins

Antagonists as provided herein also can be small organic molecules, such as those natural products, or those compounds synthesized by conventional organic synthesis or combinatorial organic synthesis. Compounds can be tested for their ability to bind to an integrin, e.g., αvβ3, αvβ1, or any others, e.g. those listed in Table II, for example by using the affinity-purification technique described herein.

Antagonists as provided herein also can be non-peptidic compounds, including, for example, oligonucleotides. “Oligonucleotides,” as used herein, refer to any heteropolymeric material containing purine, pyrimidine and other aromatic bases. DNA and RNA oligonucleotides are suitable for use with the invention, as are oligonucleotides with sugar (e.g., 2′ alkylated riboses) and backbone modifications (e.g. phosphorothioate oligonucleotides). Oligonucleotides may present commonly found purine and pyrimidine bases such as adenine, thymine, guanine, cytidine and uridine, as well as bases modified within the heterocyclic ring portion (e.g., 7-deazaguanine) or in exocyclic positions. “Oligonucleotide” also encompasses heteropolymers with distinct structures that also present aromatic bases, including polyamide nucleic acids and the like.

An oligonucleotide antagonist as provided herein can be generated by a number of methods known to one of skill in the art. In one embodiment, a pool of oligonucleotides is generated containing a large number of sequences. Pools can be generated, for example, by solid phase synthesis using mixtures of monomers at an elongation step. The pool of oligonucleotides is sorted by passing a solution containing the pool over a solid matrix to which αvβ3 or fragment thereof has been affixed. Sequences within the pool that bind to the αvβ3 are retained on the solid matrix. These sequences are eluted with a solution of different salt concentration or pH. Sequences selected are subjected to a second selection step. The selected pool is passed over a second solid matrix to which αvβ3 has been affixed. The column retains those sequences that bind to αvβ3, thus enriching the pool for sequences specific for αvβ3. The pool can be amplified and, if necessary, mutagenized and the process repeated until the pool shows the characteristics of an antagonist of the invention. Individual antagonists can be identified by sequencing members of the oligonucleotide pool, usually after cloning said sequences into a host organism such as E. coli.

Identification of Antagonists of Integrins and Other Target Proteins

Antagonists of αvβ3 have been described in U.S. Publication No. 2003/0113331; U.S. Publication No. 2004/242490 A1; WO 2004/073649; U.S. Publication No. 2004/224896 A1, and; WO 2004/087734. Antagonists of β1 integrins are discussed in “Integrin Receptors and the Regulation of Angiogenesis,” Rodrigueq, D., and Brooks, P., Chapter 5, The New Antiotherapy, eds. Fan and Kohn, Humana Press, Totowa, N.J. 2002; and Drake, C. J. et al., Dev. Dyn., 1992, 193:83-91, both incorporated herein by reference. Antagonists are evaluated for their ability to bind an integrin, and furthermore can be evaluated for their ability to inhibit binding the integrin to its ligand. Measurement of binding of antagonists to an integrin, and their ability to inhibit binding of the integrin to other molecules, including its natural ligands, can be accomplished, e.g., using an enzyme-linked-immunosorbent assay (ELISA), described in the publications listed above and herein. The ELISA is commonly used and well-known to those of skill in the art.

The ELISA also can be used to identify compounds which exhibit increased specificity for an integrin in comparison to other molecules including other integrins. The specificity assay is conducted by running parallel ELISAs in which a potential antagonist is screened concurrently in separate assay chambers for the ability to bind the integrin. Another technique for measuring apparent binding affinity familiar to those of skill in the art is a surface plasmon resonance technique (analyzed on a BIACORE 2000 system) (Liljeblad, et al., Glyco. J. 2000, 17:323-329). Standard measurements and traditional binding assays are described by Heeley, R. P., Endocr. Res. 2002, 28:217-229.

Antagonists of integrins and other target molecules, e.g. those listed in Tables I-XVI, can also be identified by their ability to compete for binding with an antagonist useful in the present invention. For example, putative antagonists can be screened by monitoring their effect on the affinity of a known antagonist, such as antibody LM609, described, e.g., in U.S. Publication No. 2005/0002936. Such antagonists likely have the same specificity as, and recognize the same epitope, as the antibody itself. Putative antagonists selected by such a screening method can bind either to an integrin, target protein or to the known antagonist. Antagonists can be selected from the putative antagonists by conventional binding assays to determine those that bind to the target protein but not to the known antagonist.

Antagonists can also be identified by their ability to bind to a solid matrix containing an integrin or other target protein. Such putative antagonists are collected after altering solution conditions, such as salt concentration, pH, temperature, etc. The putative antagonists are further identified by their ability to pass through, under appropriate solution conditions, a solid matrix to which αvβ3 has been affixed.

Antagonists useful in the compositions and methods provided herein also can be assayed for their ability to influence tumor development processes, e.g., angiogenesis, tumor metastasis, cell adhesion, cell migration, cell proliferation, and tumor growth in a tissue. Any suitable assay known to one of skill in the art can be used to monitor such effects. Several such techniques are described herein.

Methods for Identifying Proteins Modulated by Antagonists of αvβ3 Integrin

Modulation of protein levels, including those of IGFBP-4 and TSP-1, can be measured using methods described in the literature and well-known to those of skill in the art. Enzyme Linked Immunosorbent Assay (ELISA), Western Blot analysis, radioimmunoassay and immunoprecipitation are examples of methods that can be used to detect and quantitate the proteins of interest. Enzymatic assays, also well known in the art, can also be used where appropriate.

Proteins that are modulated at least 1.5 to 2-fold (up or down) are preferred for use in the methods provided herein. For example, as determined by ELISA and described in Example IX, levels of TSP-1 were found to be increased in conditioned medium (CM) from cells lacking αvβ3 (ECVL and M21 L) by nearly 2 to 4 fold as compared to CM from cells expressing αvβ3 (ECV and M21).

As described in Example XV, ELISA showed that the relative levels of IGFBP-4 increased in CM from ECVL by greater than 10-fold as compared to ECV.

Administered Products of Genes

Gene products or proteins identified and administered according to the methods of the invention include TSP-1 and IGFBP-4. Also contemplated for administration are polypeptide portions of IGFBP-4, wherein the portion of the gene product is an active portion having angiogenesis, metastasis or tumor development-inhibiting properties, or it has the ability to exert a beneficial effect on angiogenesis-dependent conditions. IGFBP-4 has been shown to be proteolyzed (see, e.g., Overgaard, J. Biol. Chem. 2000, 275(40):31128-33). It has been reported in the literature that a number of proteins that inhibit angiogenesis, including angiostatin, endostatin, pexstatin, tumstatin, laminin, and fibronectin, have increased anti-angiogenic activity when present in cleaved forms as compared to full-length forms. The resulting cleavage products possess anti-angiogenic activity. For example, the angiogenesis inhibitor, angiostatin, is derived from plasminogen, and the prothrombin kringle-2 domain is a cleavage product of prothrombin (Lee, et al., J. Biol. Chem. 1998, 273 (44):28805-12; Soff, G. A., Cancer Metastasis Rev. 2000, 19(1-2):97-107). A short peptide from matrix metalloproteinase-2 (MMP-2) has also been found to inhibit angiogenesis and tumor growth (U.S. Pub. No. 2002/0182215 A1, incorporated herein by reference in its entirety). Therefore, identified polypeptides, as well as naturally-occurring cleavage products, are contemplated for use according to the methods of the invention. The use of cryptic regions of ECM components having anti-angiogenic function are discussed in, e.g., Schenk, S., et al., Trends in Cell Biol. 2003, 13: 366-375 and Kalluri, R. Nat. Rev. Cancer 2003, 3: 422-433.

Gene products can be expressed from genes identified according to the methods of the invention by numerous methods known to those of skill in the art and described in the literature.

For example, recombinantly-produced proteins of the present invention can be directly expressed or expressed as fusion proteins. The recombinant protein can be purified by a combination of cell lysis (e.g., sonication, French press) and affinity chromatography. For fusion products, subsequent digestion of the fusion protein with an appropriate proteolytic enzyme can release the desired recombinant protein.

Polynucleotides containing genes identified using the methods of the present invention may be cloned, using standard cloning and screening techniques, from a cDNA library, (see for instance, Sambrook, et al., Molecular Cloning: A Laboratory Manual, 2nd Ed., Cold Spring Harbor Laboratory Press, Cold Spring Harbor, N.Y. (1989)). These polynucleotides can also be obtained from natural sources such as genomic DNA libraries or can be synthesized using well known and commercially available techniques.

When genes are used for the recombinant production of gene products or proteins, the polynucleotide including the gene sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. Polynucleotides can also contain non-coding 5′ and 3′ sequences, such as transcribed, non-translated sequences, splicing and polyadenylation signals, ribosome binding sites and sequences that stabilize mRNA.

There are a number of methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Modifications of the technique, exemplified by the Marathon technology (Clontech Laboratories Inc.) for example, have significantly simplified the search for longer cDNAs. In the Marathon technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene-specific and adaptor-specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al., 1989. Preferred methods of introducing polynucleotides into host cells include, for instance, calcium phosphate transfection, DEAE-dextran mediated transfection, transfection, micro-injection, cationic lipid-mediated transfection, electroporation, transduction, scrape loading, ballistic introduction or infection.

Representative examples of appropriate hosts include, e.g., bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

As understood in the art, a great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., 1989. Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

The proteins provided herein, recombinant or synthetic, can be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). The protein may then be isolated from cells expressing the protein and further purified by standard protein chemistry techniques.

Methods of Assaying Tumor Metastasis

Tumor metastasis can be measured by a number of techniques known to those of skill in the art and published in the literature. The Examples describe assaying tumor metastasis using the chick embryo model (Brooks et al., Meth. Mol. Biol. 1999, 129:257-269; Testa et al., Cancer Res 1999, 59:3812-3820), and the murine model (Vantyghem, et al., Cancer Res 2003, 63:4763-4765). Subsequent histological and immunofluorescence analyses can be performed as described in the literature (Brooks, et al., Cell 1996, 85:683-693; Brooks, et al., Science 1994, 264:569-571).

Methods of Assaying Angiogenesis

Methods of measuring alterations in angiogenesis are well known in the art. For example, angiogenesis can be measured in the chick chorioallantoic membrane (CAM), in a method referred to as the CAM assay. The CAM assay has been described in detail by others and has been used to measure both angiogenesis and neovascularization of tumor tissues. See Ausprunk et al., Am. J. Pathol., 1975, 79:597-618 and Ossonski et al., Cancer Res. 1980, 40:2300-2309. The CAM assay is a well-recognized assay model for in vivo angiogenesis because it involves the neovascularization of whole tissue with chick embryo blood vessels growing into either the CAM or into the tissue grown on the CAM.

The CAM assay is particularly useful because the system includes an internal control for toxicity. The health of the embryo indicates toxicity since the chick embryo itself is exposed to test reagents.

Another method for measuring alterations in angiogenesis is the in vivo rabbit eye model, referred to as the rabbit eye assay. The rabbit eye assay has been described in detail by others and has been used to measure both angiogenesis and neovascularization in the presence of angiogenic inhibitors such as thalidomide. See D'Amato et al., Proc. Natl. Acad. Sci. 1994, 91:4082-4085.

The rabbit eye assay is a well recognized assay model for in vivo angiogenesis because the neovascularization process, exemplified by rabbit blood vessels growing from the outer rim of the cornea into the cornea, is easily visualized through the naturally transparent corneal membrane. Additionally, both the extent and the amount of stimulation/regression of neovascularization can easily be monitored over time. Finally, this method has an additional benefit of indicating toxicity of the test reagent. Since the rabbit is exposed to test reagents, the health of the rabbit is an indication of toxicity of the test reagent.

Another assay, referred to as the chimeric mouse assay, measures angiogenesis in the chimeric mouse:human mouse model. This assay is described herein, and in detail by others, as a method for measuring angiogenesis, neovascularization, and regression of tumor tissues. See Yan, et al., J. Clin. Invest. 1993, 91:986-996.

The chimeric mouse assay is a useful in vivo model for angiogenesis because the transplanted skin grafts closely resemble normal human skin histologically. Additionally, neovascularization of whole tissue is occurring wherein human blood vessels are growing from grafted human skin into human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of new vessel growth. Furthermore, it is easy to monitor effects on the growth of any tissue transplanted upon the grafted skin, such as a tumor tissue. Finally, the assay is useful because there is an internal control for toxicity in the assay system. The health of the mouse is an indication of toxicity when exposed to a test reagent.

To confirm the effects of a compound, e.g., IGFBP-4, on angiogenesis, the mouse Matrigel plug angiogenesis assay can be used. Various growth factors (IGF-1, bFGF or VEGF) (250 ng) and Heparin (0.0025 units per/ml) are mixed with growth factor reduced Matrigel as previously described (Montesano, et al., J. Cell Biol. 1983, 97:1648-1652; Stefansson, et al., J. Biol. Chem. 2000, 276:8135-8141). IGFBP-4 or control BSA (10 to 500 ng) can be included in the Matrigel preparations. In control experiments, Matrigel is prepared in the absence of growth factors. Mice are injected subcutaneously with 0.5 ml of the Matrigel preparation and allowed to incubate for one week. Following the incubation period, the mice are sacrificed and the polymerized Matrigel plugs surgically removed. Angiogenesis within the Matrigel plugs is quantified by two established methods, including immunohistochemical analysis and hemoglobin content (Furstenberger, et al., Lancet. 2002, 3:298-302; Volpert, et al., Cancer Cell 2002, 2(6):473-83; Su, et al., Cancer Res. 2003, 63:3585-3592). For immunohistochemical analysis, the Matrigel plugs are embedded in OCT, snap frozen and 4 μm sections prepared. Frozen sections are fixed in methanol/acetone (1:1). Frozen sections are stained with polyclonal antibody directed to CD31. Angiogenesis is quantified by microvascular density counts within 20 high powered (200×) microscopic fields.

Hemoglobin content can be quantified as described previously (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164). The Matrigel implants are snap frozen on dry ice and lyophilized overnight. The dried implants are resuspended in 0.4 ml of 1.0% saponin (Calbiochem) for one hour, and disrupted by vigorous pipetting. The preparations are centrifuged at 14,000 g for 15 minutes to remove any particulates. The concentration of hemoglobin in the supernatant is then determined directly by measuring the absorbency at 405 nm and compared to a standard concentration of purified hemoglobin. This method of quantification has been used successfully and has been shown to correlate with angiogenesis (Schnaper, et al., J. Cell Physiol. 1993, 256:235-246; Montesano, et al., J. Cell Biol. 1983, 97:1648-1652; Stefansson, et al., J. Biol. Chem. 2000, 276:8135-8141; Gigli, et al., J. Immunol. 1986, 100:1154-1164).

Methods of Assaying Cell Adhesion

Cell adhesion can be measured by methods known to those of skill in the art. Assays have been described previously, e.g. by Brooks, et al., J. Clin. Invest 1997, 99:1390-1398. For example, cells can be allowed to adhere to substrate (i.e., an ECM component) on coated wells. Non-attached cells are removed by washing, and non-specific binding sites are blocked by incubation with BSA. The attached cells are stained with crystal violet, and cell adhesion is quantified by measuring the optical density of eluted crystal violet from attached cells at a wavelength of 600 nm.

Methods of Assaying Cell Migration

Assays for cell migration have been described in the literature, e.g., by Brooks, et al., J. Clin. Invest 1997, 99:1390-1398 and methods for measuring cell migration are known to those of skill in the art. In one method for measuring cell migration described herein, membranes from transwell migration chambers are coated with substrate (here, thermally denatured collagen), the transwells washed, and non-specific binding sites blocked with BSA. Tumor cells from sub-confluent cultures are harvested, washed, and resuspended in migration buffer in the presence or absence of assay antibodies. After the tumor cells are allowed to migrate to the underside of the coated transwell membranes, the cells remaining on the top-side of the membrane are removed and cells that migrate to the under-side are stained with crystal violet. Cell migration is then quantified by direct cell counts per microscopic field.

Methods of Assaying Tumor Growth

Tumor growth can be assayed by methods known to those of skill in the art, e.g., as described in Xu, et al., J. Cell Biol 2001, 154:1069-1079. An assay for chick embryo tumor growth can be performed as follows: single cell suspensions of CS1 melanoma (5×10⁶ per embryo) or HT1080 fibrosarcoma (4×10⁵ per embryo) are applied in a total volume of 40 μl of RPMI to the CAMs of 10-day-old embryos (Brooks et al., 1998). Twenty four hours later, the embryos receive a single intravenous injection of an inhibitor of αvβ3, e.g., LM609, or control molecule (100 μg per embryo). For example, if an antibody inhibitor is used, an isotype-matched antibody can serve as a control. Tumors are grown for 7 days, then resected and wet weights are determined. Experiments can be performed with five to ten embryos per condition.

Another method for assaying tumor growth makes use of the SCID mouse, as follows:

Subconfluent human M21 melanoma cells are harvested, washed, and resuspended in sterile PBS (20×10⁶ per ml). SCID mice are injected subcutaneously with 100 μl of M21 human melanoma cell (2×10⁶) suspension. Three days after tumor cell injection, mice are either untreated or treated intraperitoneally (100 μg/mouse) with either Mab LM609 or an isotype-matched control antibody. The mice are treated daily for 24 days. Tumor size is measured with calipers and the volume estimated using the formula V=(L²×W)/2, where V is equal to the volume, L is equal to the length, and W is equal to the width.

Methods of Assaying Cell Proliferation

Cell proliferation can be assayed by methods known to those of skill in the art. As described herein, subconfluent human endothelial cells (HUVECs) can be resuspended in proliferation buffer containing low (5.0%) serum in the presence or absence of CM (25 μl) from ECV or ECVL cells, and endothelial cells allowed to proliferate for 24 hours. Proliferation can be quantified by measuring mitochondrial dehydrogenase activity using a commercially available WST-1 assay kit (Chemicon).

Methods for Administering Gene Product to a Patient

The dosage ranges for the administration of the product of a gene that is modulated by the specific binding of an antagonist to αvβ3, or fragment thereof, depend upon the form of the gene product, and its potency, and are amounts large enough to produce the desired effect wherein angiogenesis, tumor metastasis, tumor growth, cell adhesion, cell proliferation, or cell migration are inhibited, or wherein the effect is favorable for treatment of an angiogenesis-dependent condition. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of the protein or polypeptide, e.g., a portion of the gene product having angiogenesis-, tumor metastasis-, tumor growth-, cell adhesion- or cell migration-inhibiting properties, sufficient to produce a measurable inhibition of angiogenesis, tumor metastasis, tumor growth, cell adhesion or cell migration in the tissue being treated or to have an effect on an angiogenesis-dependent condition. Inhibition of these symptoms can be measured according to methods described herein, or by other methods known to one skilled in the art. Methods for assessing the effect on an angiogenesis-dependent condition will depend on the condition being treated, and for the particular condition, such methods will be known to those of skill in the art.

It is to be appreciated that the potency, and therefore an expression of a “therapeutically effective” amount can vary. However, as shown by the present assay methods, one skilled in the art can readily assess the potency of a gene product of this invention. Potency can be measured by a variety of means, including, but not limited to: the measurement of inhibition of angiogenesis in the CAM assay, in the in vivo rabbit eye assay, or in the in vivo chimeric mouse:human assay; the inhibition of tumor metastasis in the chick embryo model or in the murine model; the inhibition of cell adhesion in a cell adhesion assay; or the inhibition of cell migration in a cell migration assay, the inhibition of tumor growth in the chick embryo assay or the SCID mouse assay, all as described herein and in the literature and known to those of skill in the art, and the like assays.

A “therapeutically effective” amount of IGFBP-4 can be determined by prevention or amelioration of adverse conditions or symptoms of diseases, injuries or disorders being treated. For all the indications of use of IGFBP-4, the appropriate dosage will of course vary depending upon, for example, the tumor type and stage and severity of the disease disorder to be treated and the mode of administration. For example, tumor inhibition as a single agent may be achieved at a daily dosage of from about to 0.1 mg/kg to 40 mg/kg body weight, preferably from about 0.2 mg/kg to about 20 mg/kg body weight of a binding protein of the invention. In larger mammals, for example, humans, as indicated daily dosage is from about 0.25 to about 5 mg/kg/day or about 70 mg per day for an average adult at a dose of 1 mg/kg/day conveniently administered parenterally, for example once a day. Dosage ranges for IGFBP-3 are described in U.S. Publication No. 2004/0127411, incorporated herein by reference.

The proteins or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, proteins or polypeptides of the invention can be administered intravenously, intraperitoneally, intramuscularly, subcutaneously, intracavity, transdermally, and can be delivered by peristaltic means.

Therapeutic compositions are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with the protein or polypeptide as described herein, dissolved or dispersed therein as an active ingredient. In a preferred embodiment, the therapeutic protein or polypeptide composition is not immunogenic when administered to a mammal or human patient for therapeutic purposes.

As used herein, the terms “pharmaceutically acceptable,” “physiologically tolerable,” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, etc. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Examples of such additional liquid phases are glycerin, vegetable oils such as cottonseed oil, and water-oil emulsions.

In further embodiments, the invention enables any of the foregoing methods to be carried out in combination with other therapies such as, for example, treatment with another compound, e.g., an inhibitor of angiogenesis and tumor development processes (e.g., a monoclonal antibody that binds to the cryptic collagen epitope, HUIV26), chemotherapy or radiation therapy, or treatment with cytotoxic agents. Chemotherapeutic agents useful in the methods of the present invention include, e.g., taxanes (i.e., Taxol, Docetaxel, Paclitaxel), dacarbazine (DTIC), Adriamycin, Bleomycin, Gemcitabine, Cyclophosphamide, Oxaliplatin, Camptothecan, Ironotecan, Fludarabine, Cisplatin and Carboplatin.

An angiogenesis inhibitor may be administered to a patient in need of such treatment before, during, or after chemotherapy. It is also preferred to administer an angiogenesis inhibitor to a patient as a prophylaxis against metastases after surgery on the patient for the removal of solid tumors.

The dosage ranges for the administration of the denatured collagen antagonist depend upon the form of the antagonist, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage also can be adjusted by the individual physician in the event of any complication.

A therapeutically effective amount is an amount of denatured collagen antagonist sufficient to produce a measurable inhibition of angiogenesis in the tissue being treated, i.e., an angiogenesis-inhibiting amount. Inhibition of angiogenesis can be measured in situ by immunohistochemistry, as described herein, or by other methods known to one skilled in the art.

Potency of a denatured collagen antagonist can be measured by a variety of means including inhibition of angiogenesis in the CAM assay, in the in vivo rabbit eye assay, in the in vivo chimeric mouse:human assay and the like assays.

A therapeutically effective amount of a denatured collagen antagonist of this invention in the form of a monoclonal antibody is typically an amount such that when administered in a physiologically tolerable composition is sufficient to achieve a plasma concentration of from about 0.01 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

Where the antagonist is in the form of a fragment of a monoclonal antibody, the amount can readily be adjusted based on the mass of the fragment relative to the mass of the whole antibody. A preferred plasma concentration in molarity is from about 2 micromolar (μM) to about 5 millimolar (mM) and preferably about 100 μM to 1 mM antibody antagonist.

A therapeutically effective amount of a denatured collagen antagonist of this invention in the form of a polypeptide, or small molecule, is typically an amount of polypeptide such that when administered in a physiologically tolerable composition is sufficient to achieve a plasma concentration of from about 0.1 microgram (μg) per milliliter (ml) to about 200 μg/ml, preferably from about 1 μg/ml to about 150 μg/ml. Based on a polypeptide having a mass of about 500 grams per mole, the preferred plasma concentration in molarity is from about 2 micromolar (μM) to about 5 millimolar (mM) and preferably about 100 μM to 1 mM polypeptide antagonist. Stated differently, the dosage per body weight can vary from about 0.1 mg/kg to about 300 mg/kg, and preferably from about 0.2 mg/kg to about 200 mg/kg, in one or more dose administrations daily, for one or several days.

The monoclonal antibodies or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule.

Thus, antagonists including monoclonal antibodies, polypeptides, and derivatives thereof can be administered intravenously, intraperitoneally, intramuscularly, subcutaneously, intracavity, transdermally, topically, intraocularly, orally, intranasally and can be delivered by peristaltic means.

The therapeutic compositions containing a monoclonal antibody or a polypeptide of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

In one embodiment, the denatured collagen antagonist is administered in a single dosage intravenously.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful. Methods for formulating injectable preparations of either antibodies or chemotherapeutic agents are well known to those of skill in the art.

The compositions are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

### EXAMPLES

Elements of the present application are illustrated by the following examples, which should not be construed as limiting in any way.

### Example I

### Expression of αvβ3 Enhanced Tumor Growth In Vivo

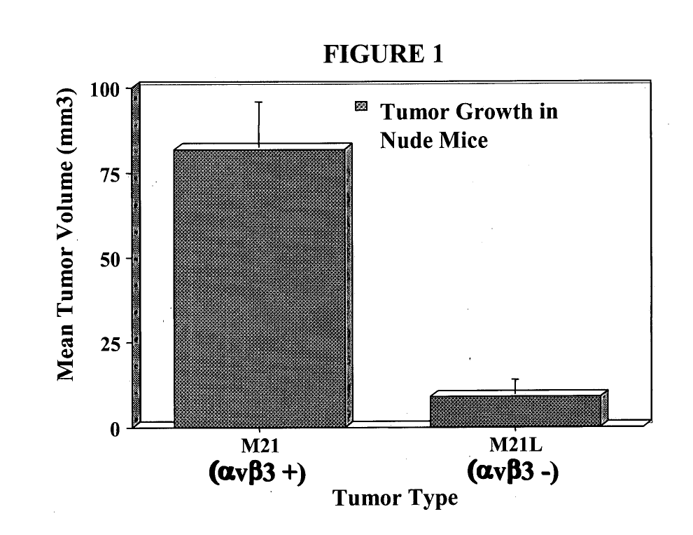

To further investigate the effect of αvβ3 on tumor growth, human melanoma cell variants (M21 or M21L) were injected subcutaneously in nude mice using methods performed similarly to methods previously described (Gasparini et al. “Vascular integrin alpha (v) beta 3: a new prognostic indicator in breast cancer.” Clin. Cancer Res. 11: 2625-2634 (1998)). Tumor growth was monitored by caliper measurements on day 7. As shown in FIG. 1, αvβ3-expressing M21 cells formed tumors that were approximately 9-fold larger (P<0.05) than tumors from cells that lacked αvβ3 (M21L). These findings confirmed previously reported results and suggest that functional expression of αvβ3 may provide a growth advantage in vivo.

### Example II: Isolation of αvβ3 Expression Variants of Human ECV304 Bladder Carcinoma Cells

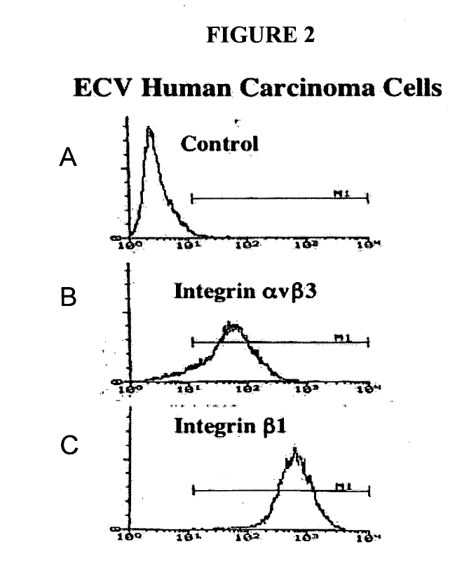

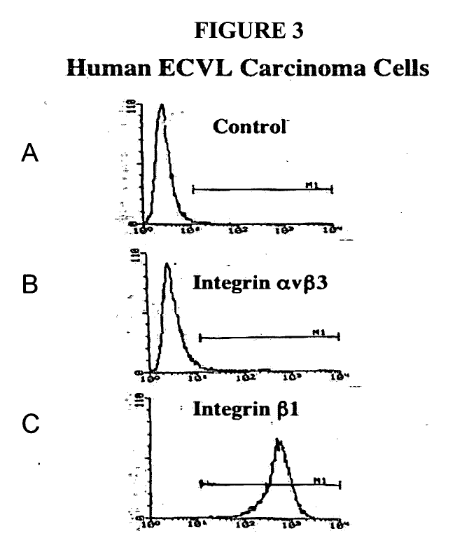

To examine the functional significance of αvβ3 on tumor growth in a histologically distinct tumor type, we isolated variants of the human bladder carcinoma cell line ECV304 that either expressed (ECV) or lacked expression (ECVL) of αvβ3. To isolate these variants, ECV cells we subjected to Fluorescence Activated Cell Sorting (FACS) of cells stained with Mab LM609 directed to αvβ3 integrin. ECV cells were incubated with Mab LM609 and FACS sorted. ECV cells that failed to express cell surface αvβ3 were expanded. The negative FACS selection procedure was carried out a total of 4 times to ensure a stable population of αvβ3 negative ECV cells. As shown in FIG. 2, the parent ECV carcinoma cells expressed high surface levels of αvβ3 (middle panel) and β1 integrins (bottom panel). In contrast, negatively-selected (ECVL) cells (see FIG. 3) expressed no detectable αvβ3 on the cell surface (middle panel). Reduction of αvβ3 expression in these cells resulted in little if any change in β1 integrin expression (bottom panel).

### Example III

### Expression of αvβ3 Enhanced Human Carcinoma Growth In Vivo but Not In Vitro

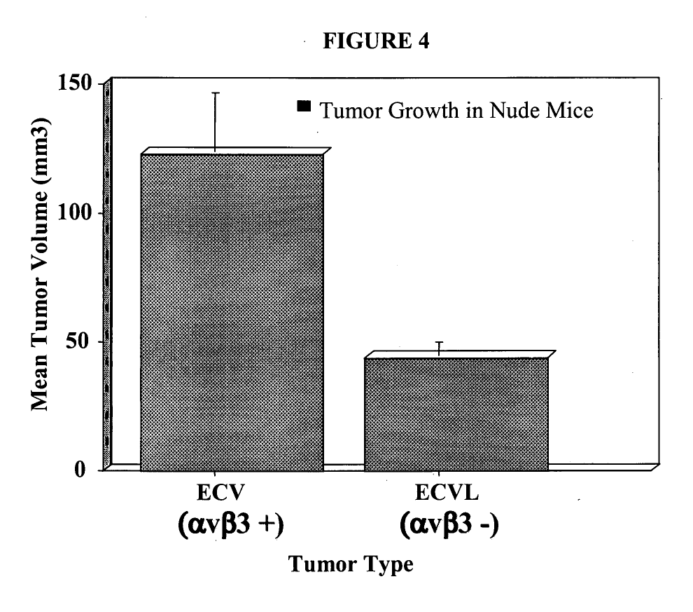

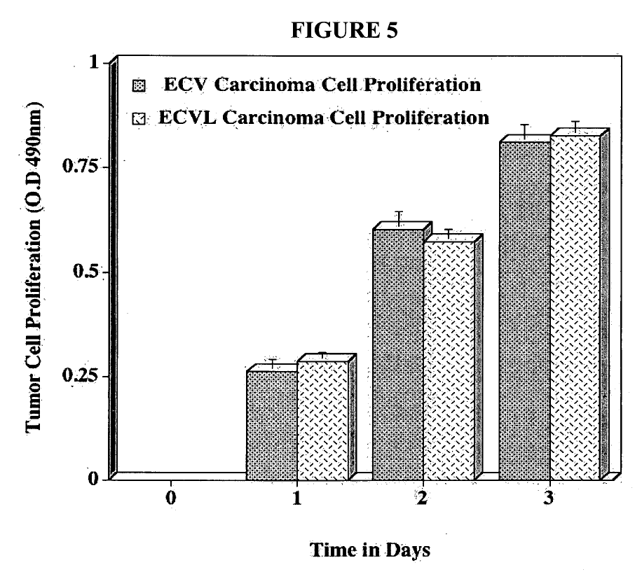

To examine whether loss of αvβ3 cell-surface expression altered tumor growth in vivo, human ECV and ECVL cells were injected subcutaneously in nude mice. Tumor growth was monitored with caliper measurements on day 14 following tumor cell inoculation. As shown in FIG. 4, αvβ3 expressing ECV cells formed tumors that were approximately 3-fold larger than ECVL cells lacking αvβ3. Given the possibility that expression of αvβ3 within ECV cells directly impacts cellular proliferation, thereby contributing to the increase in tumor size, we compared the proliferative capacities of ECV and ECVL cells in vitro. Equal numbers of cells were allowed to proliferation for 3 days. As shown in FIG. 5, little if any change in proliferation was detected between ECV and ECVL cells in vitro. These findings agree with previously published data using M21 and M21L cells (Gasparini et al. “Vascular integrin alpha (v) beta 3: a new prognostic indicator in breast cancer.” Clin. Cancer Res. 11: 2625-2634 (1998)).

### Example IV

### Elevated Angiogenesis Associated with Tumors Expressing Integrin αvβ3

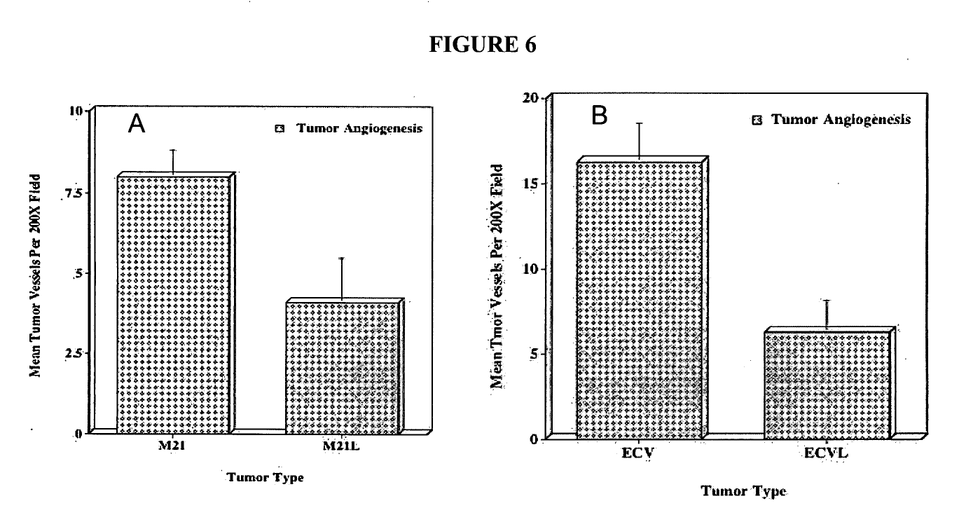

To evaluate the possibility that the growth advantage of αvβ3 expressing tumors (M21 or ECV) may be associated with an increase in angiogenesis, tumors from mice were harvested and tumor angiogenesis was analyzed. Frozen sections of tumors were stained with a polyclonal antibody directed to CD31. The number of CD31-expressing blood vessels per 200× microscopic field were determined using methods previously described (Gasparini et al. “Vascular integrin alpha (v) beta 3: a new prognostic indicator in breast cancer.” Clin. Cancer Res. 11: 2625-2634 (1998)). As shown in FIGS. 6A and 6B, αvβ3-expressing tumors (M21 and ECV) exhibited a significant (P<0.05) 2.0 to 2.5-fold increase in the number of blood vessels as compared to tumors lacking αvβ3 (M21 L and ECVL). These findings suggest that αvβ3 modulates angiogenesis within these tumors.

### Example V

### CS1 Melanoma Tumors Expressing αvβ3 Exhibited Enhanced Blood Flow

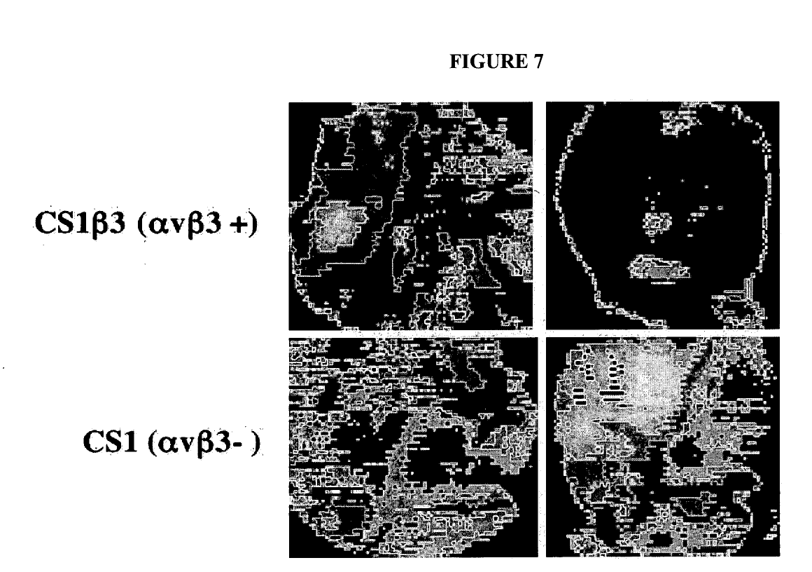

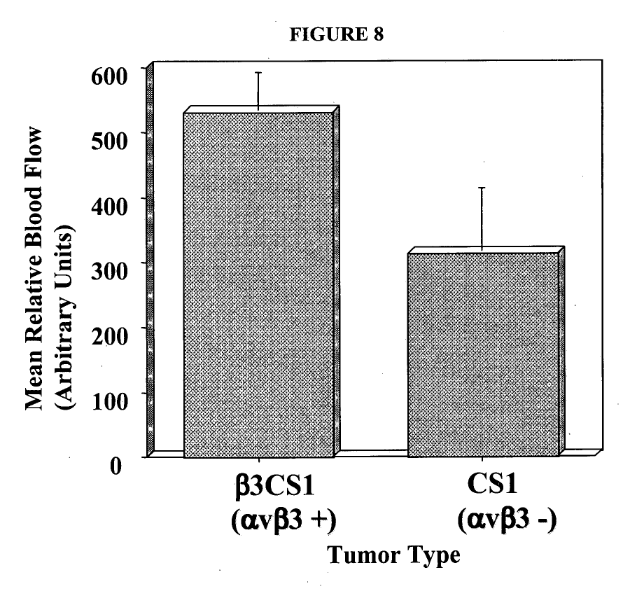

To study the potential role of αvβ3 in tumor angiogenesis in a third model, the relative tumor blood flow was examined in vivo using laser Doppler imaging. CS1 cell variants that either express (CS1β3) or lack (CS1) αvβ3 have been described previously (Brooks et al. “Insulin-like growth factor cooperates with integrin alpha v beta 5 to promote tumor cell dissemination in vivo.” J. Clin. Invest. 99: 1390-1398 (1997)). CS1 cell variants were inoculated on the CAMs to 10-day old chick embryos (Petitclerc et al. “New Functions for non-collagenous domains of human collagen type-IV: novel integrin ligands inhibiting angiogenesis and tumor growth in vivo.” J. Biol. Chem. 275: 8051-8061 (2000)). Tumors were allowed to grow for a total of 7 days and the relative tumor blood flow was examined by laser Doppler scanning (Rai and Gulati. “Evidence for the involvement of ET(B) receptors in ET-1-induced changes in blood flow to the rat breast tumor.” Cancer. Chemother. Pharmacol. 51: 21-28 (2002); Jacob et al. “Laser Doppler flux-metry in laryngeal squamous cell carcinoma.” Clin. Otolaryngol. 28: 24-28 (2003); and Stanton et al. “Expansion of Microvascular bed and increased solute flux in human basal cell carcinoma in vivo, measured by fluorescein video angiography.” Cancer Res. 63:3969-3979 (2003)). As shown in FIG. 7, CS1β3 tumors were associated with elevated levels of blood flow (red color) as compared to CS1 tumors. In fact, CS1β3 tumors were associated with an approximately 40% increase in blood flow as compared to CS1 tumors (P<0.05) that lacked αvβ3 (see FIG. 8).

### Example VI

### Inhibition of Angiogenesis In Vivo by Conditioned Medium (CM) from Tumor Cells Lacking αvβ3

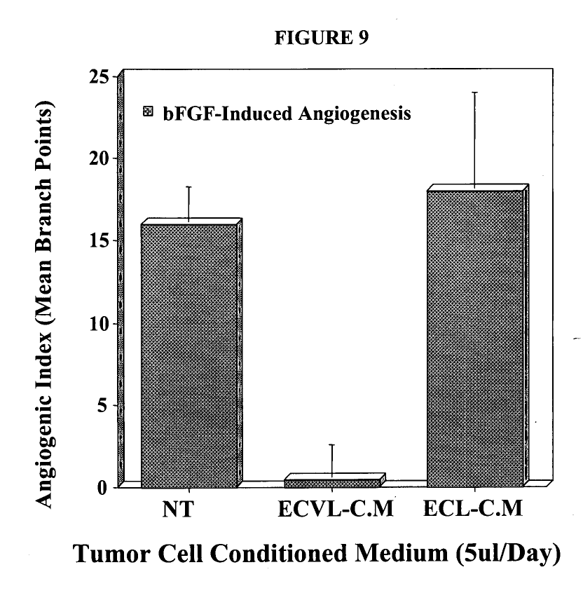

To investigate the possibility that αvβ3 may regulate angiogenesis by modulating expression of angiogenesis inducers, inhibitors, or a combination of both, concentrated serum-free conditioned media (CM) from equal numbers of tumor cells expressing (M21 and ECV) or lacking (M21L and ECVL) αvβ3 were examined for their effects on bFGF-induced angiogenesis. Filter discs containing bFGF were placed on the chorioallantoic membranes (CAMs) of 10-day old chick embryos (Brooks et al. “Use of the 10-day old chick embryo model for studying angiogenesis.” Meth. Mol. Biol. 129: 257-269 (1999)). Twenty-four hours later, the embryos were treated topically (40 μl/day) with CM. At the end of a 3-day incubation period the CAMs were removed and angiogenesis quantified (Brooks et al. “Use of the 10-day old chick embryo model for studying angiogenesis.” Meth. Mol. Biol. 129: 257-269 (1999)). As shown in FIG. 9, CM from ECVL cells significantly (P<0.001) inhibited bFGF-induced angiogenesis by greater than 90% as compared to control. CM from ECV cells had no significant effect (P>0.300) on angiogenesis. In similar studies, CM from M21L cells also (P<0.01) inhibited bFGF-induced angiogenesis by greater than 90%, while CM from M21 cells had only minimal effects on angiogenesis. Taken together, these findings suggest that αvβ3 regulates expression of a secreted inhibitor of angiogenesis.

### Example VII

### Inhibition of Endothelial Cell Proliferation In Vitro by CM from Tumor Cells Lacking αvβ3

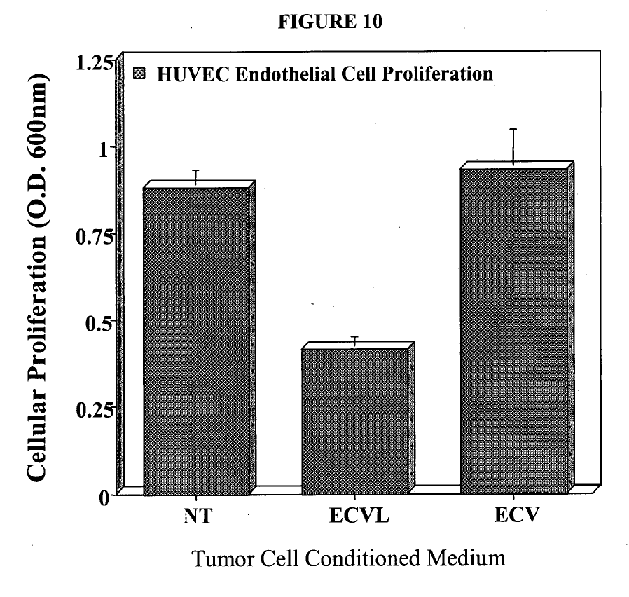

To assess the effects of tumor cell CM on endothelial cell proliferation in vitro, subconfluent human endothelial cells (HUVECs) were resuspended in proliferation buffer containing low (5.0%) serum in the presence or absence of CM (251 μl) from ECV or ECVL cells. Endothelial cells were allowed to proliferate for 24 hours. Proliferation was quantified by measuring mitochondrial dehydrogenase activity using the commercially available WST-1 assay kit. As shown in FIG. 10, CM from ECVL cells inhibited HUVEC cell proliferation by approximately 50%, while CM from ECV cells had no effect.

### Example VIII

### Inhibition of Tumor Growth In Vitro by CM from Tumor Cells Lacking αvβ3

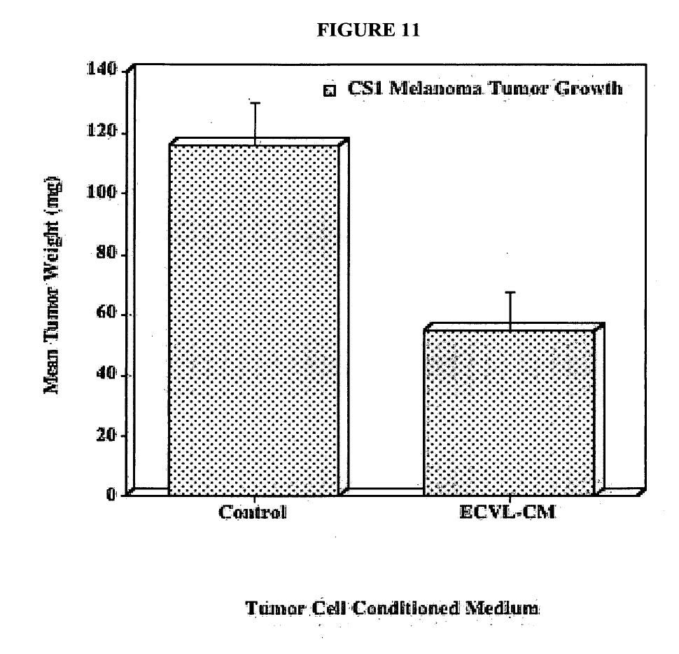

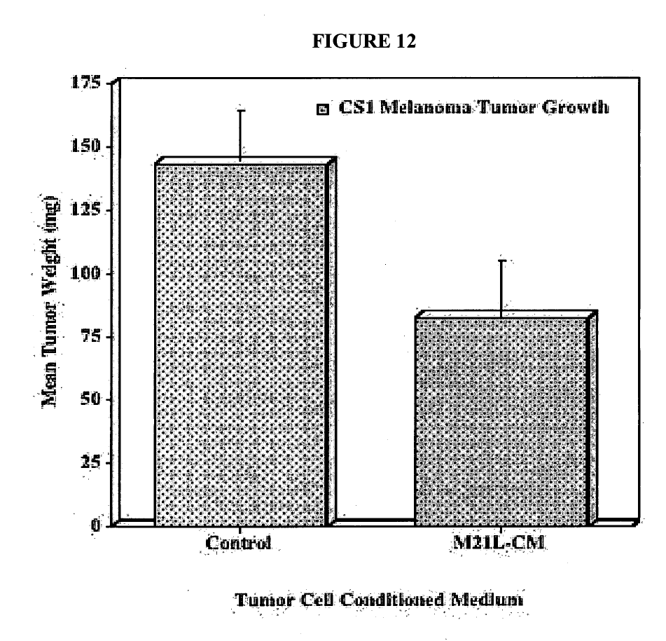

To examine the effects of CM from tumor cells that lacked expression (M21L and ECVL) of αvβ3 on tumor growth in vivo, CS1 tumors were seeded on the CAMs of 10-day old chick (Brooks et al. “Disruption of angiogenesis by PEX, a non-catalytic metalloproteinase fragment with integrin binding activity.” Cell. 92: 391-400 (1998)). The embryos were treated daily by topical addition (25 μl/day) of CM from either M21L or ECVL cells. At the end of a 7-day treatment period the resulting tumors were removed and wet weights determined. As shown in FIGS. 11 and 12, daily treatments with CM from either M21L or ECVL tumor cells resulted in a significant decrease (P<0.05) in tumor weight by approximately 50% compared to controls.

### Example IX

### Elevation in Levels of TSP-1 in CM from Tumor Cells Lacking αvβ3

To investigate the potential mechanisms by which αvβ3 may regulate angiogenesis, an Affymetrix™-based differential cDNA array analysis was performed on ECV cells that either expressed (ECV) or lacked (ECVL) αvβ3.

To perform the cDNA array analysis, either ECV or ECVL tumor cells were resuspended in serum-free media and added to plates (7×10⁶ cells per plate). The cells were allowed to incubate for a total of 12 hours. Following the 12-hour incubation period, the cells were harvested and the RNA was isolated using both a TRIzol reagent and the Qiagen Rneasy Mini Protocol for RNA Cleanup. After RNA extraction, the amount and quality of RNA was quantified utilizing a spectrophotomer.

Total RNA (5-8 μg) was utilized to synthesize double-stranded cDNA. The first cDNA strand was obtained using a reaction mixture containing a T7-(dT) 24 Primer, 1× First Strand Buffer, 0.1 M DTT and 10 mM dNTP mix in addition to the extracted RNA. The tubes were incubated at 42° C. for approximately 1.5 hours. For the second-strand cDNA synthesis, a 1× Second Strand Buffer, 10 mM dNTP mix, 10 U/ml of E. coli DNA Ligase, 10 U/ml of DNA Polymerase I and RNaseH were added and allowed to incubate at 16° C. for 2.5 hours.

Following the incubation period, T4 DNA Polymerase was added and the tubes were incubated for 5 min and stored at −80° C. The final double-stranded cDNA product was cleaned utilizing phenol extraction and ethanol precipitation. Next, the synthesized cDNA was converted to cRNA and labeled with biotin labeled ribonucleotides in a reaction mixture that also included HY Reaction Buffer, 10×DTT, RNase Inhibitor Mix and 20×RNA Polymerase.

The final cRNA product was cleaned utilizing the Qiagen Rneasy Mini Protocol for RNA Cleanup and 15 μg of cRNA was fragmented and hybridized to a U95Av2 chip.

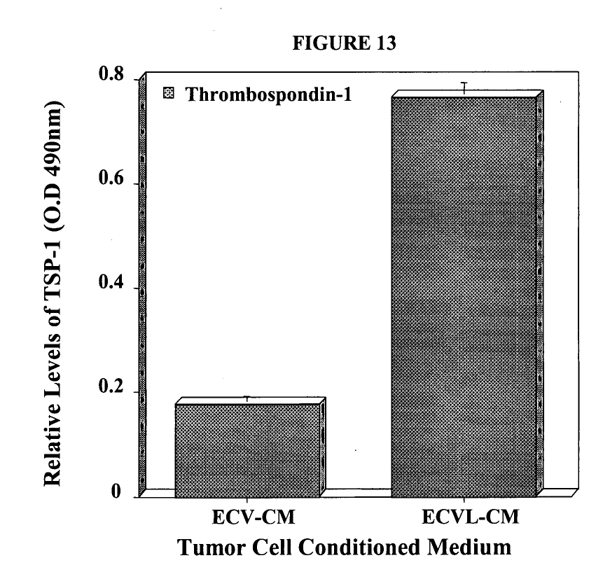

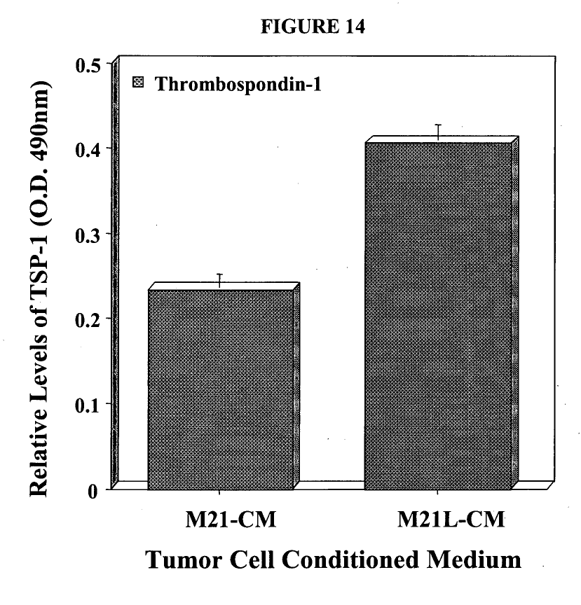

A number of genes within these cells were differentially expressed. Among the genes exhibiting significant up-regulation in ECVL as compared to ECV cells, the endogenous angiogenesis inhibitor thrombospondin-1 (TSP-1) was increased by approximately 7-fold. Based on the cDNA array, we analyzed the serum free CM from ECV and M21 cell variants for TSP-1 by solid phase ELISA. As shown in FIGS. 13 and 14, the relative levels of TSP-1 were found to be increased in CM from ECVL and M21L by nearly 2 to 4 fold as compared to CM from ECV and M21.

### Example X

### Reduction of ECVL CM Antiproliferative Activity by Immune Depletion of TSP-1

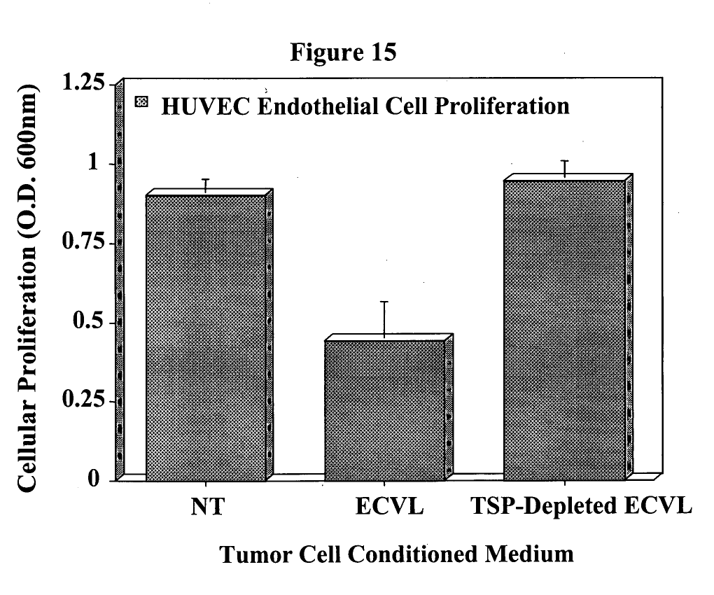

To assess the effects of TSP-1 within the CM of ECVL cells on endothelial cell behavior, we immune depleted TSP-1 from ECVL CM with a Mab directed to TSP-1. CM from ECVL cells was incubated (1 hour) with an anti-TSP-1 Mab followed by incubation with Protein A sepharose beads and the immune complexes were removed by centrifugation. The immune-depletion procedure was carried out 4 times to ensure reduction in TSP-1 levels. The effects of TSP-1 depleted and control depleted ECVL CM on endothelial cell proliferation was carried out as described above. As shown in FIG. 15, control-depleted ECVL conditioned medium inhibited HUVEC proliferation by approximately 50% as compared to no treatment. In contrast, CM from ECVL cells that was depleted of TSP-1 exhibited little if any effects on HUVEC cell proliferation. These data suggest that the presence of elevated levels of TSP-1 in the CM contributes to its ability to inhibit angiogenesis.

### Example XI

### Regulation of IGFBP-4 and TSP-1 by siRNA-Mediated Reduction in β3 Integrin

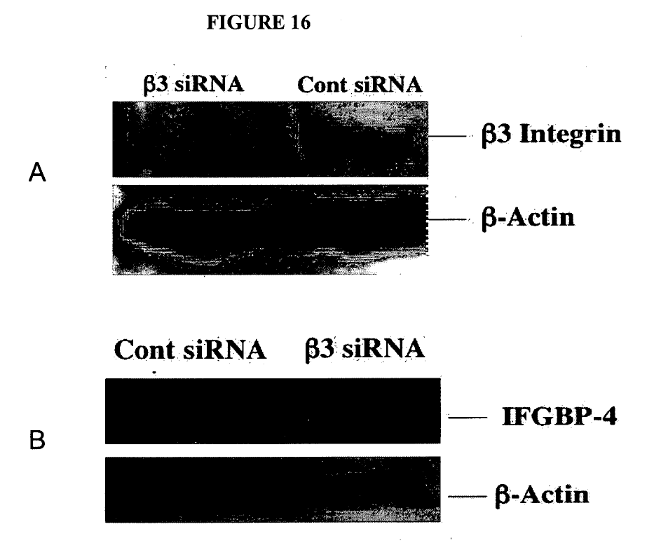

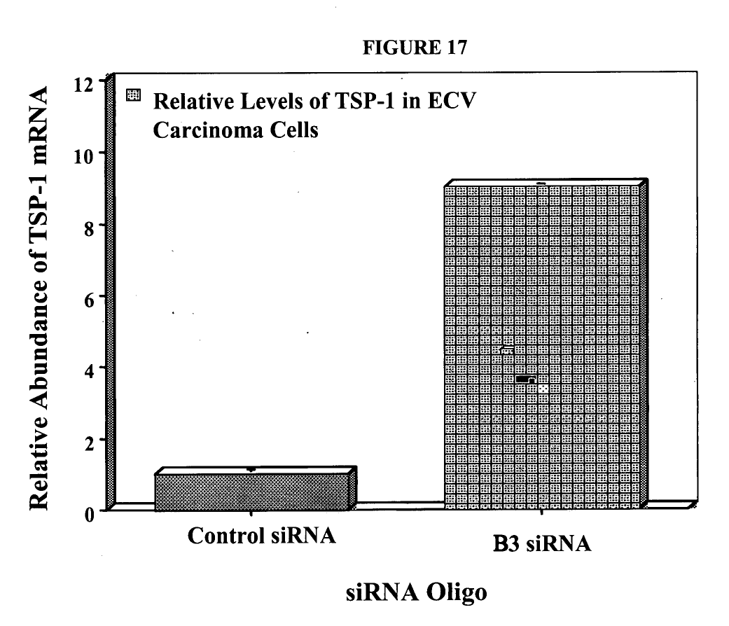

To further study effects of integrin αvβ3 on angiogenesis, we reduced expression of β3 integrin in M21 and ECV cells using siRNA. β3-specific or non-specific siRNA oligos were transfected into tumor cells. Transfectants were isolated, expanded and cell lysates or RNA was prepared for analysis by either Western blot (FIGS. 16A and B) or real time PCR (FIG. 17). As shown in FIG. 16A, the level of β3 integrin was reduced by greater than 70% in β3 siRNA transfected cells as compared to controls, while no change in β-Actin or β1 integrins (data not shown) was observed. In contrast, expression of IGFBP-4 was increased (>60%) in β3 siRNA transfected cells as compared to control cells (FIG. 16B). The relative levels of TSP-1 were significantly elevated in β3 siRNA transfected ECV cells in which β3 integrin is significantly reduced compared to control transfected cells (FIG. 17). Taken together, these findings suggest that αvβ3 expression and/or ligation may suppress expression of TSP-1 and IGFBP-4.

### Example XII

### αvβ3-Mediated Cellular Interactions Suppressed Expression of TSP-1

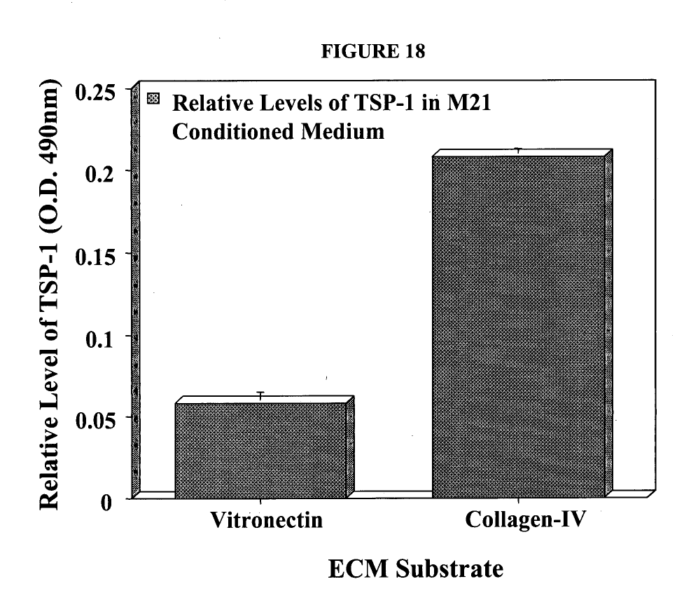

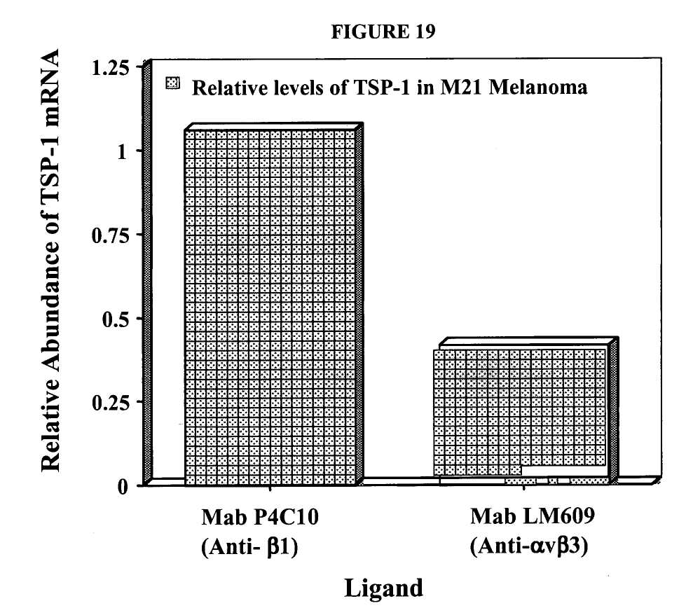

Integrin αvβ3 is known to bind the ECM protein vitronectin but does not bind to triple helical collagen type-IV. Therefore, we assessed the effects of M21 cell interactions with vitronectin on TSP-1 expression in comparison to intact collagen type-IV. M21 cells were allowed to interact with either vitronectin or intact collagen type-IV for 48 hours and the CM was collected and concentrated. The relative level of TSP-1 was assessed within the CM as described above. As shown in FIG. 18, CM from M21 cells interacting with the non-αvβ3 ECM ligand collagen type-IV exhibited an approximately 4 fold increase in TSP-1 as compared to CM from cells interacting with the known αvβ3 ligand vitronectin. Since other αv integrins can also interact with vitronectin, we examined TSP-1 expression in cells specifically interacting with the anti-integrin Mabs known to initiate signaling via distinct integrin receptors (Stromblad et al. “Suppression of p53 activity and P21WAF1/CIP1 expression by vascular integrin αvβ3 during angiogenesis.” J. Clin. Invest. 98: 426-433 (1996); Stromblad et al. “Suppression of p53 activity and P21WAF1/CIP1 expression by vascular integrin αvβ3 during angiogenesis.” J. Clin. Invest. 98: 426-433 (1996)). As shown in FIG. 19, the relative levels of TSP-1 in cells ligating αvβ3 was reduced by greater than 50% as compared to cells ligating β1 integrins as measured by real time PCR. Importantly, the relative levels of TSP-1 was normalized to cells attached to non-integrin ligand (poly-Lysine).

### Example XII

### Inhibition of αvβ3 Ligation Up-Regulated TSP-1

To further examine the effect of blocking αvβ3-mediated interactions on TSP-1, we evaluated TSP-1 expression in cells interacting with the known αvβ3 ligand denatured collagen type-IV. Relative expression of TSP-1 was assessed by both real time quantitative RT-PCR and Western Blot analysis. Tumor cells (M21) were allowed to interact with denatured collagen type-IV in the presence or absence of Mab LM609 or an isotype matched control antibody, and mRNA and whole cell lysates were prepared.

Real Time quantitative RT-PCR was carried out essentially as described with some modifications (Livak et al., Method 2001, 25:402-408). Total RNA was isolated using RNeasy miniprep columns (Qiagen, Valencia Calif.) according to the manufacturer's instructions. Total RNA (1 μg) was reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection was carried out using an ABI Prism 7900 Sequence Detection System. Reactions were carried out in microAmp 96 well reaction plates. Primers and probes were designed using Primer 3 version 2 and ENSEMBL software (Promega).

The following human-specific real time PCR primer pairs were used to detect TSP-1:

| | |
|---|---|
| 5′-TCCAAAGCGTCTTCACCAG-3′ | (SEQ ID NO: 37) |
| and | |
| 5′-GAGACAGCCTTTGTTGCTGAG-3′. | (SEQ ID NO: 38) |

The primers used to detect control gene β2-macroglobulin were:

(SEQ ID NO: 39)

5′-AAAGATGAGTATGCCTGCCG-3′ (forward),
and

(SEQ ID NO: 40)

5′-CCTCCATGATGATGGTGCTTACA-3′ (reverse).

cDNA from samples were labeled with SYBR Green (Roche) and real time PCR was run using a Light Cycler (NYU Genomic Core Services). Quantification of data was performed using Light Cycler real time PCR analysis software package 3.5 (Roche).

Fold induction was calculated using methods described by Livak et al. (2001). Amplification products utilized through Sybergreen detection were initially checked by electrophoresis on ethidium bromide stained agarose gels. The estimated size of the amplified products matched the calculated size for transcript by visual inspection.

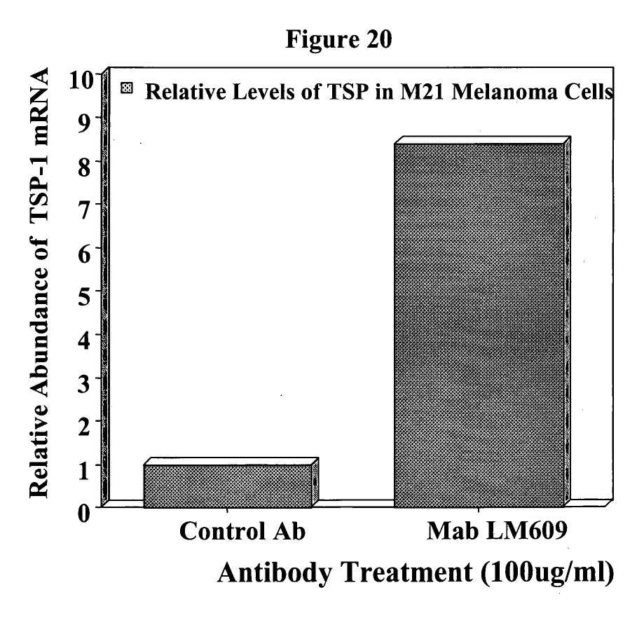

As shown in FIG. 20, the relative level of TSP-1 RNA was elevated by approximately 8-fold in M21 cells treated with the anti-αvβ3 specific Mab LM609 compared to an isotype matched control antibody as measured by real time PCR. These data suggest that specific inhibition of αvβ3 may increase expression of TSP-1 in vitro and in vivo.

### Example XIV

### Inhibition of αvβ3 Ligation Up-Regulated IGFBP-4

To examine the effects that blocking αvβ3-mediated interactions has on IGFBP-4 we evaluated the effects of Mab LM609 on IGFBP-4 expression in cells interacting with the known αvβ3 ligand denatured collagen type-IV. M21 cells were allowed to interact with denatured collagen type-IV in the presence or absence of Mab LM609 or an isotype matched control antibody for 12 hours, and levels of IGFBP-4 RNA were measured by PCR as described with regard to measurement of TSP-1 RNA.

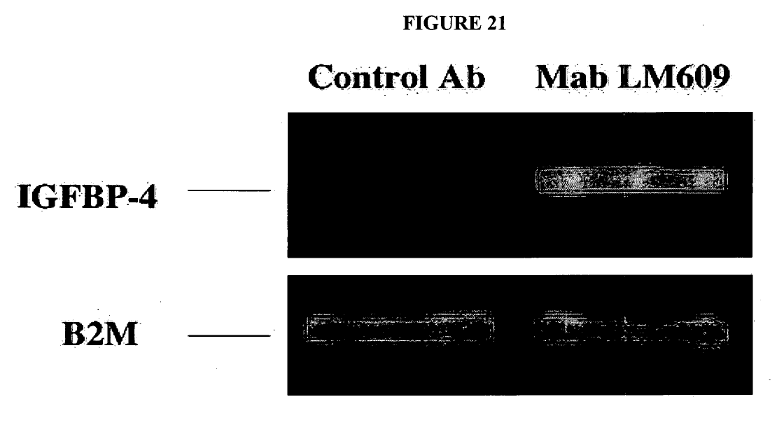

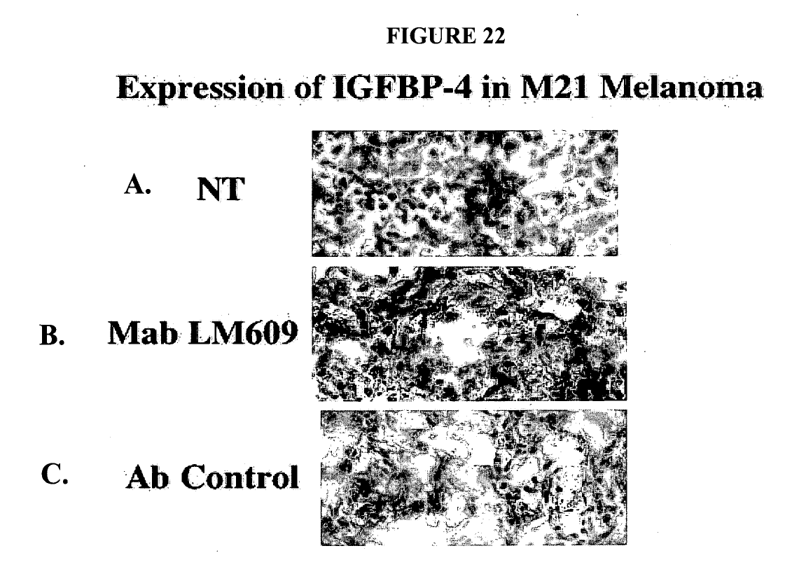

As shown in FIG. 21, the relative level of IGFBP-4 was elevated by approximately 8-fold in M21 cells treated with the anti-αvβ3 specific Mab LM609 compared to an isotype matched control antibody. IGFBP-4 RNA was measured by real time PCR as described with regard to measurement of TSP-1 RNA. As shown in FIGS. 21 and 22, expression of IGFBP-4 was significantly enhanced in M21 cells (FIG. 21) and M21 tumors grown in the chick embryo following treatment with Mab LM609 (FIG. 22). These data suggest that specific inhibition of αvβ3 may increase expression of IGFBP-4 in vitro and in vivo.

### Example XV

### Elevated Levels of IGFBP-4 Protein in Conditioned Medium from Tumor Cells Lacking αvβ3

Differential cDNA array analysis suggested increased expression of IFGBP-4 in ECVL as compared to ECV cells. The Affymetrix™-based differential cDNA array analysis was performed similarly to that described in Example I, comparing ECV and ECVL cells.

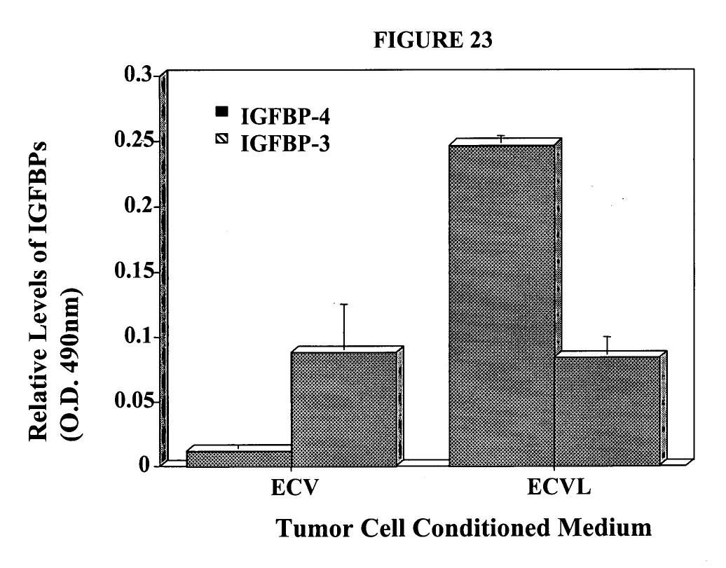

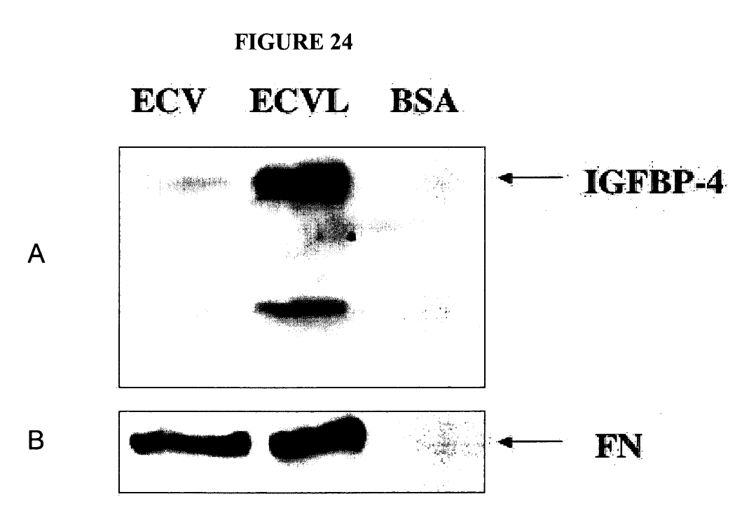

The relative levels of IGFBP-4 were analyzed in conditioned medium (CM) from ECV and ECVL cells by solid phase ELISA (FIG. 23) and Western blot (FIG. 24). As shown by ELISA, the relative levels of IGFBP-4 increased in CM from ECVL by greater than 10-fold compared to ECV, while little if any change in the levels of IGFBP-3 was observed. As shown in FIG. 24, Western Blot analysis showed that IGFBP-4 was dramatically increased in the CM of ECVL cells as compared to ECV cells while little if any change was detected in soluble fibronectin.

### Example XVI

### Peptide Antagonist Inhibition of the Binding of αvβ3 to Tumor Cells Enhances Expression of Certain Genes

To evaluate the effect of blocking αvβ3-mediated interactions using a peptide antagonist, an Affymetrix™-based differential cDNA array analysis is performed using B16F10 tumor cells treated or not treated with the peptide antagonist.

Tumor cells (7×10⁶) are resuspended in serum-containing medium and added to plates in the presence or absence of the peptide antagonist or a control peptide, e.g., as described in U.S. 2003/0176334. The cells are allowed to incubate for a total of 12 hours. Following the 12-hour incubation period, the cells are harvested and the RNA is isolated using both a TRIzol reagent and the Qiagen Rneasy Mini Protocol for RNA Cleanup. After RNA extraction, the amount and quality of RNA is quantified utilizing a spectrophotomer. 5-8 μg of total RNA is utilized to synthesize double-stranded cDNA.

The first cDNA strand is obtained using a reaction mixture containing a T7-(dT) 24 Primer, 1× First Strand Buffer, 0.1 M DTT and 10 nM dNTP mix in addition to the extracted RNA. The tubes are incubated at 42° C. for approximately 1.5 hours. For the second strand cDNA synthesis, a 1× Second Strand Buffer, 10 mM dNTP mix, 10 U/ml of E. coli DNA Ligase, 10 U/ml of DNA Polymerase I and RNaseH are added and allowed to incubate at 16° C. for 2.5 hours.

Following the incubation period, T4 DNA Polymerase is added and the tubes are again incubated for 5 min and stored at −80° C. The final double-stranded cDNA product is cleaned utilizing phenol extraction and ethanol precipitation. Next, the synthesized cDNA is converted to cRNA and labeled with biotin labeled ribonucleotides in a reaction mixture that also includes HY Reaction Buffer, 10×DTT, RNase Inhibitor Mix and 20×RNA Polymerase. The final cRNA product is cleaned utilizing the Qiagen Rneasy Mini Protocol for RNA Cleanup and 15 μg of cRNA is fragmented and hybridized to a U95Av2 chip.

Expression levels of differential cDNA array analysis of B16F10 tumor cells treated with the peptide antagonist suggest a significant increase in the expression of certain genes.

Relative expression levels of the genes identified are assessed by both real time quantitative RT-PCR and Western Blot analysis. Tumor cells (B16F10) are allowed to interact with denatured collagen type-IV in the presence or absence of the peptide antagonist or control peptide, and mRNA and whole cell lysates are prepared for use in the analyses.

Real Time quantitative RT-PCR is carried out essentially as described with some modifications (Livak et al., Method 2001, 25:402-408). Total RNA is isolated using RNeasy miniprep columns (Qiagen, Valencia Calif.) according to the manufacturer's instructions. Total RNA (1 μg) is reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection is carried out using an ABI Prism 7900 Sequence Detection System. Reactions are carried out in microAmp 96 well reaction plates. Primers and probes are designed using Primer 3 version 2 and ENSEMBL software (Promega).

The primers used to detect control gene β2-macroglobulin are:

(SEQ ID NO: 39)

5′-AAAGATGAGTATGCCTGGGG-3′ (forward)
and

(SEQ ID NO: 40)

5′-CCTCCATGATGATGCTGCTTACA-3′ (reverse).

cDNA from samples is labeled with SYBR Green (Roche) and real time PCR run using a Light Cycler (NYU Genomic Core Services). Quantification of data is performed using Light Cycler real time PCR analysis software package 3.5 (Roche).

Fold induction is calculated using methods described by Livak et al., 2001. Amplification products utilized through Sybergreen detection are initially checked by electrophoresis on ethidium bromide stained agarose gels. The estimated size of the amplified products matches the calculated size for transcript by visual inspection.

### Example XVII

### Organic Peptide Mimetic Antagonist Inhibition of the Binding of αvβ3 to Tumor Cells Enhances Expression of Certain Genes

To evaluate the effect of inhibiting αvβ3-mediated interactions with cryptic epitopes of denatured collagen using an organic peptide mimetic antagonist, an Affymetrix™-based differential cDNA array analysis is performed using B16F10 tumor cells treated or not treated with the organic peptide mimetic antagonist.

Non-tissue culture treated dishes are coated overnight with 100 μg/ml of denatured collagen IV in PBS. The next morning the plates are washed and incubated in blocking solution (1% BSA in PBS) for approximately 30 minutes. Tumor cells (7×10⁶) are resuspended in serum-free media and added to each plate in the presence or absence of the organic peptide mimetic antagonist of αvβ3, e.g., as described in U.S. Pub. No. 2004/0063790, or a control antagonist. The cells are allowed to incubate for a total of 12 hours.

Following the 12-hour incubation period, the cells are harvested and the RNA is isolated using both a TRIzol reagent and the Qiagen Rneasy Mini Protocol for RNA Cleanup. After RNA extraction, the amount and quality of RNA is quantified utilizing a spectrophotomer, and 5-8 μg of total RNA is utilized to synthesize double-stranded cDNA. The first cDNA strand is obtained using a reaction mixture containing a T7-(dT)₂₄ Primer, 1× First Strand Buffer, 0.1 M DTT and 10 mM dNTP mix in addition to the extracted RNA. The tubes are incubated at 42° C. for approximately 1.5 hours. For the second strand cDNA synthesis, a 1× Second Strand Buffer, 10 mM dNTP mix, 10 U/ml of E. coli DNA Ligase, 10 U/ml of DNA Polymerase I and RNaseH are added and allowed to incubate at 16° C. for 2.5 hours.

Following the incubation period, T4 DNA Polymerase is added and the tubes are again incubated for 5 min and stored at −80° C. The final double-stranded cDNA product is cleaned utilizing phenol extraction and ethanol precipitation. Next, the synthesized cDNA is converted to cRNA and labeled with biotin labeled ribonucleotides in a reaction mixture that also includes HY Reaction Buffer, 10×DTT, RNase Inhibitor Mix and 20×RNA Polymerase. The final cRNA product is cleaned utilizing the Qiagen Rneasy Mini Protocol for RNA Cleanup and 15 μg of cRNA is fragmented and hybridized to a U95Av2 chip.

Expression levels of differential cDNA array analysis of B16F10 tumor cells treated with the organic peptide mimetic antagonist suggest a significant increase in the expression of certain genes.

Relative expression levels of the genes identified are assessed by both real time quantitative RT-PCR and Western Blot analysis. Tumor cells (B16F10) are allowed to interact with denatured collagen type-IV in the presence or absence of the organic peptide mimetic antagonist or control antagonist, and mRNA and whole cell lysates are prepared for use in the analyses.

Real Time quantitative RT-PCR is carried out essentially as described with some modifications (Livak et al., Method 2001, 25:402-408). Total RNA is isolated using RNeasy miniprep columns (Qiagen, Valencia Calif.) according to the manufacturer's instructions. Total RNA (1 μg) is reverse transcribed using 1× Reverse Transcriptase Buffer, MgCl₂ (3 mM), DNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection is carried out using an ABI Prism 7900 Sequence Detection System. Reactions are carried out in microAmp 96 well reaction plates. Primers and probes are designed using Primer 3 version 2 and ENSEMBL software (Promega).

The primers used to detect control gene 62-macroglobulin are:

(SEQ ID NO: 39)

5′-AAAGATGAGTATGCCTGCCG-3′ (forward)
and

(SEQ ID NO: 40)

5′-CCTCCATGATGATGCTGCTTACA-3′ (reverse).

cDNA from samples is labeled with SYBR Green (Roche) and real time PCR run using a Light Cycler (NYU Genomic Core Services). Quantification of data is performed using Light Cycler real time PCR analysis software package 3.5 (Roche).

Fold induction is calculated using methods described by Livak et al., 2001. Amplification products utilized through Sybergreen detection are initially checked by electrophoresis on ethidium bromide stained agarose gels. The estimated size of the amplified products matches the calculated size for transcript by visual inspection.

### Example XVIII

### Effect of mAb HUIV26 in Combination with CLK-Peptide on Adhesion of M21 Cells to Denatured Collagen Type IV

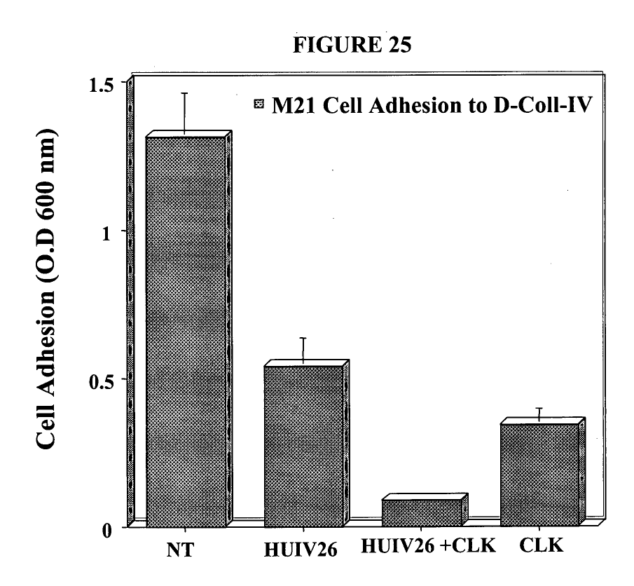

The effects of CLK-peptide, mAb HUIV26, and the combination of both on tumor cell adhesion were examined in vitro. Non-tissue culture treated 48-well microtiter plates were coated overnight at 4° C. with either native or denatured collagen type-IV (5 μg/ml). The plates were then blocked with 1% BSA in PBS for 1 hour at 37° C. Human melanoma (M21) cells at 10⁵ cells/well in adhesion buffer were allowed to attach to the coated wells in the presence of either CLK-peptide (100 μg/ml), or mAb HUIV26 (100 μg/ml, μl/well), or both together (CLK-peptide 25 μg/ml, mAb HUIV26 100 μg/ml). The cells were washed 2× with PBS, stained with crystal violet and destained with 10% acetic acid. Cell adhesion was quantified by measuring the O.D. of eluted dye at 600 nm. Data bars represent the mean O.D.±standard deviation from triplicate wells. The combination of both CLK-peptide and mAb HUIV26 had a greater effect on adhesion of M21 cells to denatured collagen type-IV than did either antagonist alone (FIG. 25), whereas neither had any effect on M21 cell adhesion to native collagen type-IV (data not shown).

### Example XIX

### CLK-Peptide Does Not Compete With Mab HUIV26 For Binding To Denatured Collagen Type IV

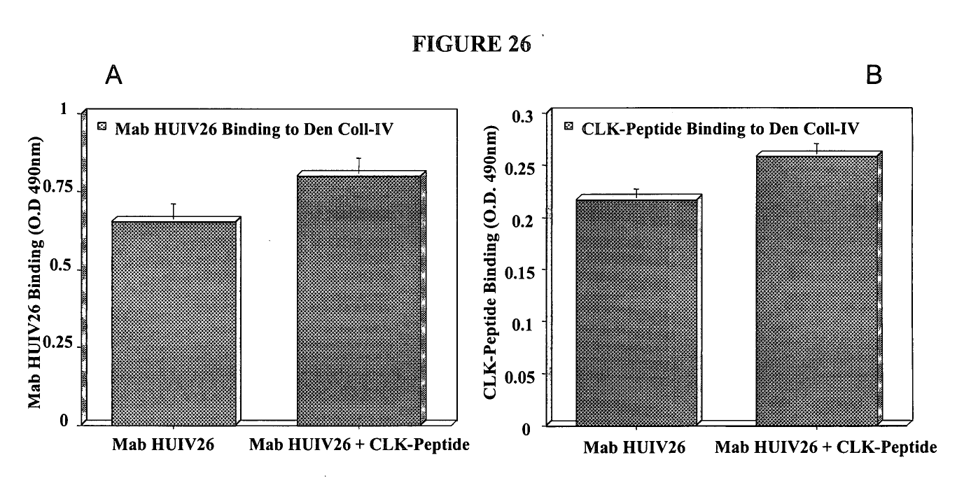

To characterize the epitope bound by CLK and HUIV26, competition ELISAs were carried out. Microtiter wells were coated with denatured collagen type-IV (1 μg/ml) overnight at 4° C., and the plates were blocked with 1% BSA for 1 hour at 37° C. Monoclonal Ab HUIV26 was added at 1 μg/ml and allowed to bind the denatured collagen in the presence or absence of 100 μg/ml of CLK-peptide for 2 hours at 37° C. Goat-anti-mouse antibody was added at 1:3000 and incubation was allowed for 1 hour at 37° C. A color solution, then a stopping solution of 4N sulfuric acid, were added and binding was quantified by measuring the O.D. 490 nm. As shown in FIG. 26, HUIV26 readily bound to denatured collagen type-IV, and CLK-peptide did not block this binding. Similar results were obtained over a wide range of peptide and antibody concentrations (1 to 100 μg/ml; data not shown). These data indicate that the CLK-peptide recognizes a cryptic epitope that is distinct from the HUIV26 cryptic collagen site.

### Example XX

### Inhibition of Binding of Labeled CLK-Peptide to Denatured Collagen Type-IV by mAb HUI77

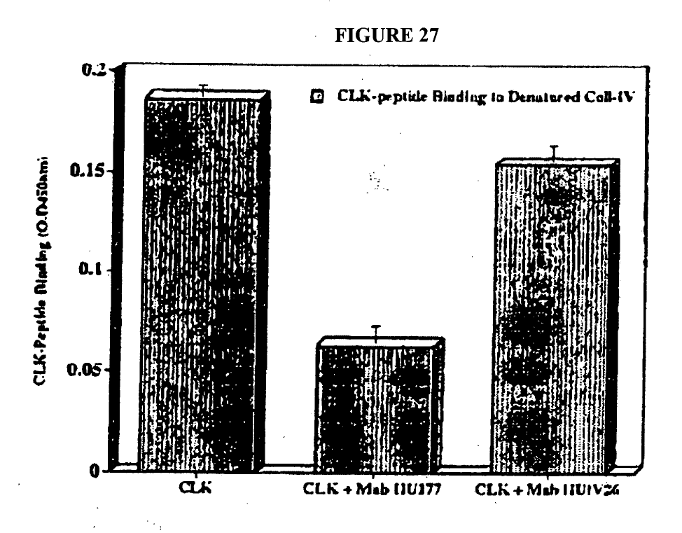

To examine the binding specificity of CLK-Peptide, the peptide was labeled and solid phase ELISAs were performed. Denatured collagen type-IV (10 μg/ml) was coated on microtiter wells and binding of either HRP-labeled CLK-Peptide or mAbs (mAbs HU177 or HUIV26) was measured by ELISA at a wavelength of 490 nm. As shown in FIG. 27, mAb HUI77, but not HUIV26, specifically inhibited CLK-binding to denatured collagen type-IV.

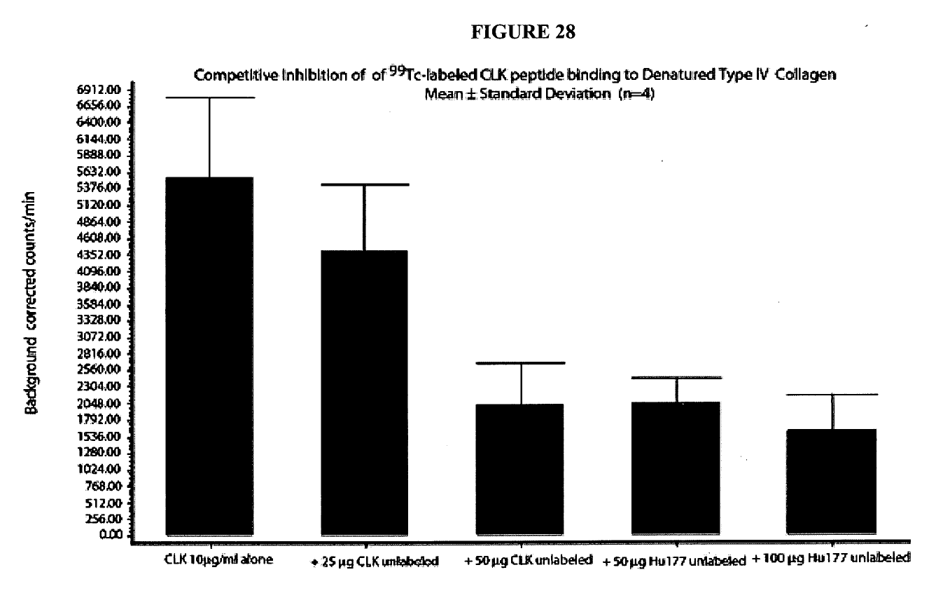

In addition, denatured collagen type-IV (10 μg/ml) was coated on microtiter wells and binding of ⁹⁹Tc-labeled CLK-Peptide in the presence or absence of unlabeled CLK-peptide or mAb HUI77 was measured using a gamma counter. As shown in FIG. 28, ⁹⁹Tc-CLK-peptide (10 μg/ml, 50 μl/well) specifically binds to denatured collagen type-IV (first bar from left). Unlabeled CLK-peptide exhibited a dose dependent inhibition of ⁹⁹Tc-CLK-peptide binding (second and third bars from left). ⁹⁹Tc-CLK-peptide was also allowed to bind to denatured collagen type-IV in the presence or absence of Mab HU177 (fourth and fifth bars from left). Mab HUI77 also inhibited ⁹⁹Tc-labeled CLK-peptide. These findings provide further evidence that the CLK-peptide binds to a similar if not identical epitope recognized by Mab HUI77.

### Example XXI

### In Vitro and In Vivo Assays to Measure Inhibitory Effect of Combination of Ant-Denatured Collagen Antibodies and Peptides with Other Agents

Cell Adhesion Assay: Human collagen (denatured or native) is coated at a concentration of 1-25 μg/mL in PBS, pH 7.4, on to 48-well non-tissue culture treated plates for 12-18 hours at 2-8° C. Non-specific sites were blocked with 1% BSA in PBS for 1 hour at room temperature. Subconfluent cultures of M21 or HUVEC cells are harvested from flasks by Versene, washed, and resuspended in adhesion buffer containing RPMI 1640, 1 mM MgCl₂, 0.2 mM MnCl₂, and 0.5% BSA. Cells are resuspended at 10⁵ cells/mL in 200 μL of adhesion buffer and preincubated for up to 1 hour at room temperature in the presence or absence of various combinations of anti-denatured collagen antibodies D93, H8, HUIV26, HUI77, XL313 at 0.1-100 μg/mL or anti-denatured collagen peptide CLK at 0.1-100 μg/mL with LM609, Vitaxin at 100 μg/mL, and added to each well and allowed to attach for 30 minutes at 37° C. The non-attached cells are removed and the attached cells are stained for 10 minutes with crystal violet. The wells are washed with PBS and cell-associated crystal violet is eluted by addition of 100 μL of 10% acetic acid and transfer to a 96-well microtiter plate. Cell adhesion is quantified by measuring the optical density at 600 nm of eluted crystal violet solution on 96-well microtiter plate reader.

Cell Proliferation Assay: Human collagens (denatured or native) are coated at a concentration of 1-25 μg/mL in PBS, pH 7.4, on to 96-well non-tissue culture treated plates for 12-18 hours at 2-8° C. Non-specific sites were blocked with 1% BSA in PBS for 1 hour at room temperature. Subconfluent cultures of M21 or HUVEC cells are harvested from flasks by Versene, washed, and resuspended in medium containing RPMI 1640, 1 mM MgCl₂, 0.2 mM MnCl₂ in the presence or absence of serum. Cells are resuspended at 10⁴-10⁵ cells/mL in 200 μL of medium and preincubated for up to 1 hour at room temperature in the presence or absence of various combinations of anti-denatured collagen antibodies D93, H8, HUIV26, HUI77, XL313 at 0.1-100 μg/mL or anti-denatured collagen peptide CLK at 0.1-100 μg/mL with LM609, Vitaxin at 100 μg/mL, and in the presence or absence of Taxol® or other chemotherapeutic agents at 1-100,000 nM and added to collagen-coated microtiter plate wells. Cells are cultured for up to 72 hours at 37° C. in a CO₂, humidified incubation. Cell proliferation is measured using a BrdU colorimetric ELISA and optical denisities measured using a 96-well plate reader. Cell proliferation is inhibited by the combinations used.

Xenogeneic Tumor Growth Assay in Mice: Subconfluent human tumor cells are harvested from tissue culture flasks, washed and resuspended in sterile PBS at 2×10⁷ cells/mL and 100 μL is injected subcutaneously in the flank of SCID or nude mice. Mice are treated once, twice/per week or daily by i.p or i.v. administration with D93, H8, HUIV26, HUI77, XL313 at 0.1-500 μg/dose or anti-denatured collagen peptide CLK at 0.01-500 μg/dose with LM609, Vitaxin at 0.1-500 μg/dose with or without the presence of Taxol® at 1-20 mg/kg at 3 days post-implantation of the tumor or when tumors reach a predetermined size (50-100 mm³). Tumors are measured with calipers and the volume estimated using the formula volume (V)=(L²×W)/2 where L=tumor length, W=perpendicular width. Tumor growth is measured daily for up to 1 month. Tumor growth is inhibited by the combinations used.

### Example XXII

### In Vitro and In Vivo Assays to Measure Inhibitory Effect of Combination of Ant-Denatured Laminin Antibodies and Peptides with Other Agents

Cell Adhesion Assay: Human laminin (denatured or native) is coated at a concentration of 1-25 μg/mL in PBS, pH 7.4, on to 48-well non-tissue culture treated plates for 12-18 hours at 2-8° C. Non-specific sites were blocked with 1% BSA in PBS for 1 hour at room temperature. Subconfluent cultures of M21 or HUVEC cells are harvested from flasks by Versene, washed, and resuspended in adhesion buffer containing RPMI 1640, 1 mM MgCl₂, 0.2 mM MnCl₂, and 0.5% BSA. Cells are resuspended at 10⁵ cells/mL in 200 μL of adhesion buffer and preincubated for up to 1 hour at room temperature in the presence or absence of various combinations of anti-laminin antibodies at 0.1-100 μg/mL or anti-denatured laminin peptide STQ at 0.1-100 μg/mL with LM609, Vitaxin at 100 μg/mL, and added to each well and allowed to attach for 30 minutes at 37° C. The non-attached cells are removed and the attached cells are stained for 10 minutes with crystal violet. The wells are washed with PBS and cell-associated crystal violet is eluted by addition of 100 μL of 10% acetic acid and transfer to a 96-well microtiter plate. Cell adhesion is quantified by measuring the optical density at 600 m of eluted crystal violet solution on 96-well microtiter plate reader.

Cell Proliferation Assay: Human laminin (denatured or native) is coated at a concentration of 1-25 μg/mL in PBS, pH 7.4, on to 96-well non-tissue culture treated plates for 12-18 hours at 2-8° C. Non-specific sites were blocked with 1% BSA in PBS for 1 hour at room temperature. Subconfluent cultures of M21 or HUVEC cells are harvested from flasks by Versene, washed, and resuspended in medium containing RPMI 1640, 1 mM MgCl₂, 0.2 mM MnCl₂ in the presence or absence of serum. Cells are resuspended at 10⁴-10⁵ cells/mL in 200 μL of medium and preincubated for up to 1 hour at room temperature in the presence or absence of various combinations of anti-denatured laminin antibodies at 0.1-100 μg/mL or anti-denatured laminin peptide STQ at 0.1-100 μg/mL with LM609, Vitaxin at 100 μg/mL, and in the presence or absence of Taxol® or other chemotherapeutic agents at 1-100,000 nM and added to laminin-coated microtiter plate wells. Cells are cultured for up to 72 hours at 37° C. in a CO₂, humidified incubation. Cell proliferation is measured using a BrdU calorimetric ELISA and optical denisities measured using a 96-well plate reader. Cell proliferation is inhibited by the combinations used.

Xenogeneic Tumor Growth Assay in Mice: Subconfluent human tumor cells are harvested from tissue culture flasks, washed and resuspended in sterile PBS at 2×10⁷ cells/mL and 100 μL is injected subcutaneously in the flank of SCID or nude mice. Mice are treated once, twice/per week or daily by i.p or i.v. administration with anti-denatured laminin antibodies at 0.1-500 μg/dose or anti-denatured laminin peptide STQ at 0.01-500 μg/dose with LM609, Vitaxin at 0.1-500 μg/dose with or without the presence of Taxol® at 1-20 mg/kg at 3 days post-implantation of the tumor or when tumors reach a predetermined size (50-100 mm³). Tumors are measured with calipers and the volume estimated using the formula volume (V)=(L²×W)/2 where L=tumor length, W=perpendicular width. Tumor growth is measured daily for up to 1 month. Tumor growth is inhibited by the combinations used.

What is claimed is:

1. A method of treating an angiogenesis-dependent condition in a mammal in need of such treatment, said method comprising administering to said mammal:
a therapeutically-effective amount of a first antagonist that specifically binds to an extracellular matrix (ECM) component;
a therapeutically-effective amount of a second antagonist that specifically binds to an integrin;
in a combination treatment regimen.

2. The method of claim 1, wherein said angiogenesis-dependent condition is selected from among an angiogenic disease, a cancer-associated disorder and a solid tumor.

3. The method of claim 1, wherein said ECM component specifically bound by said first antagonist is selected from among cryptic collagen epitopes, cryptic laminin epitopes, fibronectin, vitronectin, fibrinogen, thrombospondin, osteopontin, tenascin and vWF.

4. The method of claim 1, wherein said integrin is selected from among the integrins listed in Table II.

5. The method of claim 3, wherein said integrin is selected from among the integrins listed in Table II.

6. The method of claim 1, wherein said ECM component bound by said first and second antagonists is denatured laminin.

7. The method of claim 6, wherein said first and said second antagonists bind different or multiple sites on said denatured laminin.

8. The method of claim 1, wherein said first and said second antagonists bind the same ECM component.

9. The method of claim 1, wherein said first and said second antagonists bind different ECM components or multiple sites on the same ECM component.

10. The method of claim 1, wherein said antagonists are selected from among antibodies, antibody fragments, peptides, combinations of antibodies and peptides, and combinations of antibody fragments and peptides.

11. The method of claim 10, wherein the antibody fragment is a fragment selected from among a Fab, a F(ab′)₂, a Fv, a scFv, a Fd and a single chain binding polypeptide.

12. The method of claim 1, further comprising administering one or more chemotherapeutic agents.

13. The method of claim 1, wherein said first antagonist and said second antagonist are administered in combination sequentially or concurrently.

14. The method of claim 2, wherein said cancer is selected from among a cancer of the head neck, eye, mouth, throat, esophagus, chest, bone, lung, colon, rectum, stomach, prostate, breast, ovary, testes, thyroid, blood, kidney, liver, pancreas, brain, and central nervous system.

15. The method of claim 1, wherein the mammal is a human.

16. A method of treating an angiogenesis-dependent condition in a mammal in need of such treatment, said method comprising administering to said mammal:
a therapeutically-effective amount of a first antagonist that specifically binds to a first extracellular matrix (ECM) component;
a therapeutically-effective amount of a second antagonist that specifically binds to a second ECM component;
in a combination treatment regimen.

17. The method of claim 16, wherein said angiogenesis-dependent condition is selected from among an angiogenic disease, a cancer-associated disorder and a solid tumor.

18. The method of claim 16, wherein said ECM component specifically bound by said first or second antagonist is selected from among cryptic collagen epitopes, cryptic laminin epitopes, fibronectin, vitronectin, fibrinogen, thrombospondin, osteopontin, tenascin and vWF.

19. The method of claim 18, wherein said integrin is selected from among the integrins listed in Table II.

20. The method of claim 18, wherein said ECM component is denatured collagen.

21. The method of claim 16, wherein said antagonists are selected from among antibodies, antibody fragments, peptides, combinations of antibodies and peptides, and combinations of antibody fragments and peptides.

22. The method of claim 21, wherein the antibody fragment is a fragment selected from among a Fab, a F(ab′)₂, a Fv, a scFv, a Fd and a single chain binding polypeptide.

23. The method of claim 16, further comprising administering one or more chemotherapeutic agents.

24. The method of claim 16, wherein said first antagonist and said second antagonist are administered in combination sequentially or concurrently.

25. The method of claim 17, wherein said cancer is selected from among a cancer of the head neck, eye, mouth, throat, esophagus, chest, bone, lung, colon, rectum, stomach, prostate, breast, ovary, testes, thyroid, blood, kidney, liver, pancreas, brain, and central nervous system.

26. The method of claim 16, wherein the mammal is a human.

27. A method of treating an angiogenesis-dependent condition in a mammal in need of such treatment, said method comprising administering to said mammal:
a therapeutically-effective amount of a first antagonist that specifically binds to a first extracellular matrix (ECM) component;
a therapeutically-effective amount of a second antagonist that specifically binds to the same ECM component as said first antagonist;
in a combination treatment regimen

28. The method of claim 27, wherein said angiogenesis-dependent condition is selected from among an angiogenic disease, a cancer-associated disorder and a solid tumor.

29. The method of claim 27, wherein said ECM component specifically bound by said first or second antagonist is selected from among cryptic collagen epitopes, cryptic laminin epitopes, fibronectin, vitronectin, fibrinogen, thrombospondin, osteopontin, tenascin and vWF.

30. The method of claim 29, wherein said EMC component is an integrin selected from among the integrins listed in Table II.

31. The method of claim 27, wherein said ECM component is denatured collagen or laminin.

32. The method of claim 27, wherein said first and said second antagonists bind the same site on said ECM component or bind different sites on said same ECM component.

33. The method of claim 27, wherein said antagonists are selected from among antibodies, antibody fragments, peptides, combinations of antibodies and peptides, and combinations of antibody fragments and peptides.

34. The method of claim 33, wherein the antibody fragment is a fragment selected from among a Fab, a F(ab′)₂, a Fv, a scFv, a Fd and a single chain binding polypeptide.

35. The method of claim 27, further comprising administering one or more chemotherapeutic agents.

36. The method of claim 27, wherein said first antagonist and said second antagonist are administered in combination sequentially or concurrently.

37. The method of claim 28, wherein said cancer is selected from among a cancer of the head neck, eye, mouth, throat, esophagus, chest, bone, lung, colon, rectum, stomach, prostate, breast, ovary, testes, thyroid, blood, kidney, liver, pancreas, brain, and central nervous system.

38. The method of claim 27, wherein the mammal is a human.

39. A method of preventing, treating or managing cancer in a patient in need thereof, said method comprising, administering to said patient:
a dose of an effective amount of an ECM-component antagonist or
an antagonist that competes with the binding of said ECM-component antagonist for binding to the ECM-component; and
a dose of an effective amount of one or more other cancer therapies.

40. The method of claim 39, wherein said ECM-component antagonist is selected from among an ECM-component-binding antibody or an antigen-binding fragment thereof, or an antibody or fragment thereof that competes with said ECM-component-binding antibody for binding to the ECM-component.

41. The method of claim 39, wherein the ECM component is selected from among cryptic collagen epitopes, cryptic laminin epitopes, fibronectin, vitronectin, fibrinogen, thrombospondin, osteopontin, tenascin and vWF.

42. The method of claim 39, wherein said antagonist is selected from among a peptide, an antibody or an antigen-binding fragment thereof, a combination of an antibody and peptide, and a combination of an antibody fragment and peptide.

43. The method of claim 42, wherein the antibody fragment is a fragment selected from among a Fab, a F(ab′)₂, a Fv, a scFv, a Fd and a single chain binding polypeptide.

44. The method of claim 39, wherein said ECM-component antagonist or an antagonist that competes with said ECM-component-binding antagonist for binding to the ECM-component, is administered to said patient concurrently or sequentially with the administration of one or more other cancer therapies.

45. The method of claim 39, wherein said one or more other cancer therapies are selected from among administration of an antagonist that bind to a second ECM component, an antagonist that binds to an integrin, one or more chemotherapeutic treatment, one or more biological therapy, one or more immunotherapy, one or more radiation therapy, one or more hormonal therapy, surgery, and a combination thereof.

46. The method of claim 39, wherein said ECM component binding antagonist is an integrin.

47. The method of claim 39, wherein said cancer is selected from among a cancer of the head neck, eye, mouth, throat, esophagus, chest, bone, lung, colon, rectum, stomach, prostate, breast, ovary, testes, thyroid, blood, kidney, liver, pancreas, brain, and central nervous system.

48. The method of claim 39, wherein the patient is a human.

49. A composition, comprising:
a therapeutically effective amount of a first antagonist that specifically binds to a first ECM component and
a therapeutically effective amount of a second antagonist that specifically binds to one of the following selected from among: a second ECM component, a different site on the first ECM component, and an integrin.

50. The composition of claim 49, wherein said ECM-component antagonist is selected from among an ECM-component-binding antibody or an antigen-binding fragment thereof, and an antibody or fragment thereof that competes with said ECM-component-binding antibody for binding to the ECM-component.

51. The composition of claim 49, wherein the ECM component is selected from among a cryptic collagen epitope, cryptic laminin epitope, fibronectin, vitronectin, fibrinogen, thrombospondin, osteopontin, tenascin and vWF.

52. The composition of claim 49, wherein said antagonist is selected from among a peptide, an antibody or an antigen-binding fragment thereof, a combination of an antibody and peptide, and a combination of an antibody fragment and peptide.

53. The composition of claim 52, wherein the antibody fragment is a fragment selected from among a Fab, a F(ab′)₂, a Fv, a scFv, a Fd and a single chain binding polypeptide.

54. A composition, comprising:
a therapeutically effective amount of a first antagonist that specifically binds to a first integrin and
a therapeutically effective amount of a second antagonist
wherein the second antagonist specifically binds to one of the following selected from among: a second integrin, a different site on the first integrin, a different component of the first integrin, or a different site on the same component of the first integrin.

55. The composition of claim 54, wherein said ECM-component antagonist is selected from among an ECM-component-binding antibody or an antigen-binding fragment thereof, and an antibody or fragment thereof that competes with said ECM-component-binding antibody for binding to the ECM-component.

56. The composition of claim 54, wherein the ECM component is selected from among a cryptic collagen epitope, cryptic laminin epitope, fibronectin, vitronectin, fibrinogen, thrombospondin, osteopontin, tenascin and vWF.

57. The composition of claim 54, wherein said antagonist is selected from among a peptide, an antibody or an antigen-binding fragment thereof, a combination of an antibody and peptide, and a combination of an antibody fragment and peptide.

58. The composition of claim 57, wherein the antibody fragment is a fragment selected from among a Fab, a F(ab′)₂, a Fv, a scFv, a Fd and a single chain binding polypeptide.

59. A composition, comprising:
a therapeutically effective amount of one or more ECM-component antagonist or an antagonist that competes with the binding of an ECM component antagonist to an ECM component and
a therapeutically effective amount of one or more cancer therapies.

60. The composition of claim 59, wherein said one or more cancer therapies are an ECM-component antagonist or an antagonist that competes with the binding of an ECM component antagonist to an ECM component.

61. The composition of claim 60, wherein said ECM-component antagonists are antagonists of the same ECM component or antagonists of different ECM components.

62. The composition of claim 59, wherein said ECM-component antagonist is selected from among an ECM-component-binding antibody or an antigen-binding fragment thereof, and an antibody or fragment thereof that competes with said ECM-component-binding antibody for binding to the ECM-component.

63. The composition of claim 59, wherein the ECM component is selected from among a cryptic collagen epitope, cryptic laminin epitope, fibronectin, vitronectin, fibrinogen, thrombospondin, osteopontin, tenascin and vWF.

64. The composition of claim 59, wherein said antagonist is selected from among a peptide, an antibody or an antigen-binding fragment thereof, a combination of an antibody and peptide, and a combination of an antibody fragment and peptide.

65. The composition of claim 64, wherein the antibody fragment is a fragment selected from among a Fab, a F(ab′)₂, a Fv, a scFv, a Fd and a single chain binding polypeptide.


SEQUENCE LISTING (40 sequences)

SEQ ID NO: 1 - protein, 123 aa, Mus musculus
QVTLKETGPGILQPSQTLSLTCSFSGFSLSTSGMGVGWIRQPSGEGLEWLABIWWDDNKYYNPSLKSRLTISKDTSSNQVFLKITSVDTADTATYYCARRANYGNPYYAMDYWGQGTSVTVSS

SEQ ID NO: 2 - protein, 123 aa, Artificial
  Synthetic variable heavy chain polypeptide
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARRANYYYYYYAMDVWGQGTTVTVSS

SEQ ID NO: 3 - protein, 123 aa, Artificial
  Synthetic variable heavy chain polypeptide
QVTLKESGPALVKPTQTLTLTCTFSGFSLSTSGMGVGWIRQPPGKALEWLADIWWDDNKYYNPSLKSRLTISKDTSSNQVFLTMTNVDPVDTATYYCARRANYGNPYYAQDYWGQGTTVTVSS

SEQ ID NO: 4 - protein, 123 aa, Artificial
  Synthetic variable heavy chain polypeptide
QVTLKESGPALVKPTQTLTLTCTFSGFSLSTSGMGVGWIRQPPGKALEWLADIWWDDNKYANPSLKSRLTISKDTSSNQVFLTMTNVDPVDTATYYCARRANYGNPYYAQDYWGQGTTVTVSS

SEQ ID NO: 5 - protein, 123 aa, Artificial
  Synthetic variable heavy chain polypeptide
QVTLKESGPALVKPTQTLTLTCTFSGFSLSTPGMGVWWIRQPPGKALEWLADIWWDDNKYTNPSLKSRLTISKDTSSNQVFLTMTNVDPVDTATYYCARRANYGNPYYAQDYWGQGTTVTVSS

SEQ ID NO: 6 - protein, 123 aa, Artificial
  Synthetic variable heavy chain polypeptide
QVTLKESGPALVKPTQTLTLTCTFSGFSLSTPGMGVWWIRQPPGKALEWLADIWWDDNKYTNPSLKSRLTISKDTSKNQVVLTMTNVDPVDTATYYCARRANYGNPYYAQDYWGQGTTVTVSS

SEQ ID NO: 7 - protein, 123 aa, Artificial
  Synthetic variable heavy chain polypeptide
QVTLKESGPALVKPTQTLTLTCTFSGFSLSTPGMGVWWIRQPPGKALEWLADIWWDDNKYTNPSLKSRLTISKDTSKNQVVLTMTNMDPVDTATYYCARRANYGNPYYAQDYWGQGTTVTVSS

SEQ ID NO: 8 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 9 - protein, 113 aa, Artificial
  Synthetic variable light chain polypeptide
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQGSHVPWTFGQGTKVEIK

SEQ ID NO: 10 - protein, 112 aa, Artificial
  Synthetic variable light chain polypeptide
DIVMTQTPLSLPVTLGEPASISCRSSQSIVHSNGNTYLEWYLQKPGQSPQLLIYKVSNRFSGVPDRFSGSGSGTDFTLKISRVEAEDVGVYYCFQGSHVPWTFGQGTKVEIK

SEQ ID NO: 11 - protein, 112 aa, Artificial; identical to SEQ ID NO: 10
  Synthetic variable light chain polypeptide

SEQ ID NO: 12 - protein, 112 aa, Artificial
  Synthetic variable light chain polypeptide
DIVMTQTPLSLPVTLGEPASISCRSSQSIVSSWGNTYLEWYLQKPGQSPQLLIYKVSNRFSGVPDRFSGSGSGTDFTLKISRVEAEDVGVYYCFQGSHVPWTFGQGTKVEIK

SEQ ID NO: 13 - protein, 112 aa, Artificial
  Synthetic variable light chain polypeptide
DIVMTQTPLSLPVTPGEPASISCRSSQSIVSSWGNTYLEWYLQKPGQSPQLLIYKVSNRFSGVPDRFSGSGSGTDFTLKISRVEAEDVGVYYCFQGSHVPWTFGQGTKVEIK

SEQ ID NO: 14 - protein, 112 aa, Artificial; identical to SEQ ID NO: 13
  Synthetic variable light chain polypeptide

SEQ ID NO: 15 - protein, 120 aa, Mus musculus
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPVDGYYDAMDYWGQGTSVTVSS

SEQ ID NO: 16 - protein, 112 aa, Artificial
  Synthetic antibody variable heavy chain polypeptide
  misc_feature (99)..(99): Xaa can be any naturally occurring amino acid
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARXAAWGQGTTVTVSS

SEQ ID NO: 17 - protein, 120 aa, Artificial
  Synthetic antibody variable heavy chain polypeptide
EVQLVESGGGLVQPGGSLRLSCAASGFTFSRYWMSWVRQAPGKGLEWIGEINPDSSTINYTPSLKDRFTISRDNAKNSLYLQMSSVRAEDTAVYYCARPVDGYYDAMDPWGQGTTVTVSS

SEQ ID NO: 18 - protein, 120 aa, Artificial
  Synthetic antibody variable heavy chain polypeptide
EVQLVESGGGLVQPGGSLRLSCAASGFTFSRYWMAWVRQAPGKGLEWIGEINPDSSTINYTPSLKDRFTISRDNAKNSLYLQMSSVRAEDTAVYYCARPVDGYYDAMDPWGQGTTVTVSS

SEQ ID NO: 19 - protein, 120 aa, Artificial
  Synthetic antibody variable heavy chain polypeptide
EVQLVESGGGLVQPGGSLRLSCAASGFTFSRYWMTWVRQAPGKGLEWIGEINPDSSTANYTPYLKDRFTISRDNAKNSLYLQMSSVRAEDTAVYYCARPVDGYYDAMDPWGQGTTVTVSS

SEQ ID NO: 20 - protein, 120 aa, Artificial
  Synthetic antibody variable heavy chain polypeptide
EVQLVESGGGLVQPGGSLRLSCAASGFTFSRYWMTWVRQAPGKGLEWVGEINPDSSTANYTPYLKDRFTISRDNAKNSLYLQMNSLRAEDTAVYYCARPVDGYYDAMDPWGQGTTVTVSS

SEQ ID NO: 21 - protein, 120 aa, Artificial
  Synthetic antibody variable heavy chain polypeptide
EVQLVESGGGLVQPGGSLRLSCAASGFTFSRYWMTWVRQAPGKGLEWVAEINPDSSTANYTPYLKDRFTISRDNAKNSLYLQMNSLRAEDTAVYYCARPVDGYYDAMDPWGQGTTVTVSS

SEQ ID NO: 22 - protein, 112 aa, Mus musculus
DIVMTQSPSLLSVSAGEKVTMSCKSSQSLLNSGNQKNYLAWYQQKPGQPPKLLIYGASTRESGVPDRFTGSGSGTDFTLIISSVQAEDLAVYYCQNDHSVPYTFGGGTKLEI

SEQ ID NO: 23 - protein, 113 aa, Artificial
  Synthetic antibody variable light chain polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQDHSYPYTFGQGTKLEIK

SEQ ID NO: 24 - protein, 113 aa, Artificial
  Synthetic antibody variable light chain polypeptide
DIVMTQSPDSLAVSAGERATINCKSSQSLLNSGNQKNYLAWYQQKPGQPPKLLIYGASTRESGVPDRFSGSGSGTDFTLIISSLQAEDVAVYYCQNDHQYPYTFGQGTKLEIK

SEQ ID NO: 25 - protein, 113 aa, Artificial; identical to SEQ ID NO: 24
  Synthetic antibody variable light chain polypeptide

SEQ ID NO: 26 - protein, 113 aa, Artificial
  Synthetic antibody variable light chain polypeptide
DIVMTQSPDSLAVSAGERATINCKSSQSLLNWYNQKNYLAWYQQKPGQPPKLLIYGASTRESGVPDRFSGSGSGTDFTLTISSLQAEDVAVYYCQNDHQYPYTFGQGTKLEIK

SEQ ID NO: 27 - protein, 113 aa, Artificial
  Synthetic antibody variable light chain polypeptide
DIVMTQSPDSLAVSLGERATINCKSSQSLLNWYNQKNYLAWYQQKPGQPPKLLIYGASTRESGVPDRFSGSGSGTDFTLTISSLQAEDVAVYYCQNDHQYPYTFGQGTKLEIK

SEQ ID NO: 28 - protein, 113 aa, Artificial; identical to SEQ ID NO: 27
  Synthetic antibody variable light chain polypeptide

SEQ ID NO: 29 - protein, 12 aa, Homo sapien
CLKQNGGNFSLG

SEQ ID NO: 30 - protein, 12 aa, Home sapien
SLKQNGGNFSLC

SEQ ID NO: 31 - protein, 17 aa, Homo sapien
KGGCLKQNGGNFSLGGK

SEQ ID NO: 32 - protein, 13 aa, Homo sapien
STQNASLLSLTVC

SEQ ID NO: 33 - protein, 19 aa, Homo sapien
KGGCSTQNAQLLSLIVGKA

SEQ ID NO: 34 - protein, 18 aa, Homo sapien
KGGSTQNAQLLSLIVGKA

SEQ ID NO: 35 - protein, 9 aa, Homo sapien
IFAGDKFWR

SEQ ID NO: 36 - protein, 11 aa, Homo sapien
CIFAGDKFWRC

SEQ ID NO: 37 - DNA, 19 bp, Artificial
  Primer
tccaaagcgtcttcaccag

SEQ ID NO: 38 - DNA, 21 bp, Artificial
  Primer
gagacagcctttgttcctgag

SEQ ID NO: 39 - DNA, 20 bp, Artificial
  Primer
aaagatgagtatgcctgccg

SEQ ID NO: 40 - DNA, 23 bp, Artificial
  Primer
cctccatgatgatgctgcttaca